(19) United States
(12) Patent Application Publication
(10) Pub. No.: US 2006/0002924 A1
(43) Pub. Date: Jan. 5, 2006

(54) Conjugate of notch signalling pathway modulators and their use in medical treatment

(76) Inventors: Mark William Bodmer, Cambridge (GB); Brian Robert Champion, Cambridge (GB); Andrew Christopher Lennard, Cambridge (GB); Grahame James McKenzie, Cambridge (GB); Tamara Tugal, Cambridge (GB); George Albert Ward, Cambridge (GB)

Correspondence Address: FROMMER LAWRENCE & HAUG, 745 FIFTH AVENUE- 10TH FL., NEW YORK, NY, 10151, US

(21) Appl. No.: 11/050,346

(22) Filed: Feb. 3, 2005

Related U.S. Application Data
(63) Continuation-in-part of application No. PCT/GB03/03285, filed on Aug. 1, 2003.

(30) Foreign Application Priority Data
Aug. 3, 2002 (GB) 0218068.5
Sep. 7, 2002 (GB) 0220849.4
Sep. 10, 2002 (GB) 0220912.0
Sep. 10, 2002 (GB) 0220913.8
Jan. 7, 2003 (GB) 0300234.2
May 24, 2003 (GB) 0312062.3
Nov. 13, 2002 (WO) PCT/GB02/05137
Nov. 13, 2002 (WO) PCT/GB02/05133
Apr. 4, 2003 (WO) PCT/GB03/01525

(51) Int. Cl. A61K 39/395; C07K 16/46

(52) U.S. Cl. 424/143.1; 514/8; 530/388.22

(57) ABSTRACT

Conjugates comprising a plurality of modulators of the Notch signalling pathway chemically bound to a support structure are described. The conjugates are useful for modulation of the Notch signalling pathway and treatment of associated conditions.

### CROSS REFERENCE TO RELATED APPLICATIONS

This application is a continuation-in-part of International Application No. PCT/GB03/003285, filed on Aug. 1, 2003, published as WO 04/013179 on Feb. 12, 2004, and claiming priority to GB Application Serial Nos. 0218068.5, filed Aug. 3, 2002; 0220849.4, filed Sep. 7, 2002; 0220912.0, filed Sep. 10, 2002; 0220913.8, filed Sep. 10, 2002; 0300234.2, filed Jan. 7, 2003; and 0312062.3, filed May 24, 2003; and to International Application Nos. PCT/GB02/005133, filed Nov. 13, 2002; PCT/GB02/005137, filed Nov. 13, 2002; and PCT/GB03/001525, Apr. 4, 2003.

Reference is made to U.S. application Ser. No. 09/310,685, filed May 4, 1999; Ser. No. 09/870,902, filed May 31, 2001; Ser. No. 10/013,310, filed December 7, 2001; Ser. No. 10/147,354, filed May 16, 2002; Ser. No. 10/357,321, filed Feb. 3, 2002; Ser. No. 10/682,230, filed Oct. 9, 2003; Ser. No. 10/720,896, filed Nov. 24, 2003; Ser. Nos. 10/763,362, 10/764,415 and 10/765,727, all filed Jan. 23, 2004; Ser. No. 10/812,144, filed Mar. 29, 2004; Ser. Nos. 10/845,834 and 10/846,989, both filed May 14, 2004; Ser. No. 10/877,563, filed Jun. 25, 2004; Ser. No. 10/899,422, filed Jul. 26, 2004; and Ser. No. 10/958,784, filed Oct. 5, 2004.

All of the foregoing applications, as well as all documents cited in the foregoing applications (“application documents”) and all documents cited or referenced in the application documents are incorporated herein by reference. Also, all documents cited in this application (“herein-cited documents”) and all documents cited or referenced in herein-cited documents are incorporated herein by reference. In addition, any manufacturer's instructions or catalogues for any products cited or mentioned in each of the application documents or herein-cited documents are incorporated by reference. Documents incorporated by reference into this text or any teachings therein can be used in the practice of this invention. Documents incorporated by reference into this text are not admitted to be prior art.

### FIELD OF THE INVENTION

The present invention relates to modulation of the Notch signalling pathway.

### BACKGROUND OF THE INVENTION

International Patent Publication No WO 98/20142 describes how manipulation of the Notch signalling pathway can be used in immunotherapy and in the prevention and/or treatment of T-cell mediated diseases. In particular, allergy, autoimmunity, graft rejection, tumour induced aberrations to the T-cell system and infectious diseases may be targeted.

It has also been shown that it is possible to generate a class of regulatory T cells which are able to transmit antigen-specific tolerance to other T cells, a process termed infectious tolerance (WO98/20142). The functional activity of these cells can be mimicked by over-expression of a Notch ligand protein on their cell surfaces or on the surface of antigen presenting cells.

A description of the Notch signalling pathway and conditions affected by it may be found, for example, in our published PCT Applications as follows:

PCT/GB97/03058 (filed on 6 Nov. 1997 and published as WO 98/20142; claiming priority from GB 9623236.8 filed on 7 Nov. 1996, GB 9715674.9 filed on 24 Jul. 1997 and GB 9719350.2 filed on 11 Sep. 1997);

PCT/GB99/04233 (filed on 15 Dec. 1999 and published as WO 00/36089; claiming priority from GB 9827604.1 filed on 15 Dec. 1999);

PCT/GB00/04391 (filed on 17 Nov. 2000 and published as WO 0135990; claiming priority from GB 9927328.6 filed on 18 Nov. 1999);

PCT/GB01/03503 (filed on 3 Aug. 2001 and published as WO 02/12890; claiming priority from GB 0019242.7 filed on 4 Aug. 2000);

PCT/GB02/02438 (filed on 24 May 2002 and published as WO 02/096952; claiming priority from GB 0112818.0 filed on 25 May 2001);

PCT/GB02/03381 (filed on 25 Jul. 2002 and published as WO 03/012111; claiming priority from GB 0118155.1 filed on 25 Jul. 2001);

PCT/GB02/03397 (filed on 25 Jul. 2002 and published as WO 03/012441; claiming priority from GB0118153.6 filed on 25 Jul. 2001, GB0207930.9 filed on 5 Apr. 2002, GB 0212282.8 filed on 28 May 2002 and GB 0212283.6 filed on 28 May 2002);

PCT/GB02/03426 (filed on 25 Jul. 2002 and published as WO 03/011317; claiming priority from GB0118153.6 filed on 25 Jul. 2001, GB0207930.9 filed on 5 Apr. 2002, GB 0212282.8 filed on 28 May 2002 and GB 0212283.6 filed on 28 May 2002);

PCT/GB02/04390 (filed on 27 Sep. 2002 and published as WO 03/029293; claiming priority from GB 0123379.0 filed on 28 Sep. 2001);

PCT/GB02/05137 (filed on 13 Nov. 2002 and published as WO 03/041735; claiming priority from GB 0127267.3 filed on 14 Nov. 2001, PCT/GB02/03426 filed on 25 Jul. 2002, GB 0220849.4 filed on 7 Sep. 2002, GB 0220913.8 filed on 10 Sep. 2002 and PCT/GB02/004390 filed on 27 Sep. 2002);

PCT/GB02/05133 (filed on 13 Nov. 2002 and published as WO 03/042246; claiming priority from GB 0127271.5 filed on 14 Nov. 2001 and GB 0220913.8 filed on 10 Sep. 2002).

Each of PCT/GB97/03058 (WO 98/20142), PCT/GB99/04233 (WO 00/36089), PCT/GB00/04391 (WO 0135990), PCT/GB01/03503 (WO 02/12890), PCT/GB02/02438 (WO 02/096952), PCT/GB02/03381 (WO 03/012111), PCT/GB02/03397 (WO 03/012441), PCT/GB02/03426 (WO 03/011317), PCT/GB02/04390 (WO 03/029293), PCT/GB02/05137 (WO 03/041735) and PCT/GB02/05133 (WO 03/042246) is hereby incorporated herein by reference

Reference is made also to Hoyne G. F. et al (1999) Int Arch Allergy Immunol 118:122-124; Hoyne et al. (2000) Immunology 100:281-288; Hoyne G. F. et al (2000) Intl Immunol 12:177-185; Hoyne, G. et al. (2001) Immunological Reviews 182:215-227; each of which is also incorporated herein by reference.

The present invention seeks to provide further means and methods for modulating the Notch signalling pathway, and, in particular, (but not exclusively) for modulating immune responses. The invention also seeks to provide agents for modulating (and, especially, activating) the Notch signalling pathway with enhanced biological or therapeutic effects.

For example, the present invention seeks to provide active agents with improved activity, especially improved Notch signalling agonist activity.

### SUMMARY OF THE INVENTION

According to a first aspect of the invention there is provided a conjugate comprising a plurality of modulators of the Notch signalling pathway (preferably at least 3, preferably at least 5) bound, preferably chemically bound, to a support structure. It will be appreciated that each modulator of the Notch signalling pathway may be the same or different to the other modulator or modulators of Notch signalling in the conjugate.

According to a further aspect of the invention there is provided a conjugate comprising a plurality of modulators of the Notch signalling pathway chemically bound to a molecular support structure. It will be appreciated that the term “molecular” as used herein generally means that the support structure comprises substantially a single molecule. It will be appreciated that this is preferably distinct from, for example, solid inert supports such as beads, particles, fibers, and the like.

In addition, although chemical (covalent) linking of modulators of Notch signalling to the support structure is preferred, it will be appreciated that in certain embodiments non-chemical linking may be used. For example, in certain embodiments, adsorption coupling (eg using electrostatic or hydrophobic interactions) or affinity coupling (eg using antibodies) may be used.

Suitably the support structure has a molecular weight of between about 500 and about 10,000,000 Da, for example between about 5,000 and about 5,000,000 Da, for example between about 500 and about 500,000 Da, or for example between about 500 and 100,000 Da, for example between about 1000 and about 50,000 Da.

Suitably the support structure comprises a polymeric material (for example polyethylene glycol) or a residue thereof. In one embodiment the polymeric material may for example comprise a branched chain polyethylene glycol polymer or a residue thereof.

Preferably the support structure is not a protein or peptide material. Suitably the support structure is substantially non-immunogenic.

If desired at least one of the modulators of the Notch signalling pathway may be coupled to the support structure via a linker moiety. Such a linker may comprise any suitable group, such as, for example, an acid, basic, aldehyde, ether or ester reactive group or a residue thereof. Suitably the linker moiety may comprise, for example, a succinimidyl propionate, succinimidyl butanoate or hexanoate, N-hydroxysuccinimide, benzotriazole carbonate, propionaldehyde, maleimide or forked maleimide, biotin, vinyl derivative or phospholipid.

According to a further aspect of the invention there is provided a conjugate comprising a plurality of modulators of the Notch signalling pathway in chemically cross-linked form.

According to a further aspect of the invention there is provided the use of a construct comprising a multiplicity of bound or linked modulators of Notch signalling in the manufacture of a medicament for modulation of immune cell activity. Preferably the immune cells are peripheral immune cells such as T-cells, B-cells or APCs rather than hematopoietic cells.

In one embodiment the modulation of the immune system comprises reduction of T cell activity. For example, the modulation of the immune system may comprise reduction of effector T-cell activity, for example reduction of helper (T_{H}) and/or cytotoxic (T_{C}) T-cell activity. Suitably the modulation of the immune system may comprise reduction of a Th1 and/or or Th2 immune response.

The term “plurality” as used herein means a number being at least two, and preferably at least five, suitably at least ten, at least twenty, for example about fifty or more.

The term “multiplicity” as used herein means a number being at least three, and preferably at least five, suitably at least ten, for example at least twenty, for example about least 50 or a hundred or more.

Suitably the conjugate comprises at least three modulators of the Notch signalling pathway, for example at least four modulators of the Notch signalling pathway, for example at least five modulators of the Notch signalling pathway. In further embodiments the conjugate may comprise at least about 10, at least about 20, at least about 30, at least about 40 or at least about 50 or 100 or more modulators of Notch signalling.

Typically, for example, the conjugate may comprise from about 10 to about 100, for example about 20 to about 80, for example about 30 to about 70, for example about 40 to about 60, for example about 50 or more modulators of Notch signalling, each of which may be the same or different.

Preferably at least one of the modulators of the Notch signalling pathway is an agent capable of activating a Notch receptor, especially a human Notch receptor (Notch protein) such as human Notch1, Notch2, Notch3 or Notch4. Such an agent may be termed “an activator of Notch”, a “Notch agonist” or a “Notch receptor agonist”. Preferably the agent is capable of activating a Notch receptor in an immune cell such as a T-cell, B-cell or APC.

For example, at least one of the modulators of the Notch signalling pathway may comprise a Notch ligand or a fragment, derivative, homologue, analogue or allelic variant thereof which is capable of activating a Notch receptor.

Suitably at least one of the modulators of the Notch signalling pathway comprises a Delta or Serrate/Jagged protein or a fragment, derivative, homologue, analogue or allelic variant thereof.

In one embodiment at least one of the modulators of the Notch signalling pathway comprises a fusion protein comprising a segment of a Notch ligand extracellular domain and an immunoglobulin F_{c} segment. Such a fusion protein may be prepared, for example, as described in WO 98/20142 (Example 2).

Suitably at least one of the modulators of the Notch signalling pathway comprises a protein or polypeptide comprising a DSL or EGF-like domain or a fragment, derivative, homologue, analogue or allelic variant thereof.

Suitably at least one of the modulators of the Notch signalling pathway comprises a protein or polypeptide comprising at least one Notch ligand DSL domain and at least 1, preferably at least 2, for example at least 3 to 8 Notch ligand EGF domains.

Other agents capable of activating Notch receptors, such as peptidomimetics (especially mimetics of naturally occurring Notch ligands), antibodies and small (eg synthetic) organic molecules which are capable of activating a Notch receptor in a conjugate of the present invention are also considered to be activators of Notch.

The term “mimetic” as used herein, in relation to polypeptides or polynucleotides, includes a compound that possesses at least one of the endogenous functions of the polypeptide or polynucleotide which it mimics.

Suitably at least one of the modulators of the Notch signalling pathway comprises a Notch ligand DSL domain and preferably up to 20, suitably up to 16, for example at least 3 to 8 EGF repeat motifs. Suitably the DSL and EGF sequences are or correspond to mammalian sequences. Preferred sequences include human sequences.

In an alternative embodiment at least one of the modulators of the Notch signalling pathway comprises an antibody, for example an anti-Notch antibody, suitably an anti-human Notch antibody (eg an antibody binding to human Notch1, Notch2, Notch3 or Notch4).

Protein, polypeptide and peptide modulators of Notch signalling may typically be coupled to reactive groups of a polymer or activated polymer for example by the formation of carbon-nitrogen (C—N) linkages, carbon-oxygen (C—O) linkages, or carbon-sulfur (C—S) linkages, optionally via a linker.

For example, in one embodiment a conjugate may have the formula:
POL(-R)ₙ
wherein POL is a polymeric support structure, R represents a modulator of Notch signalling (each of which may be the same or different) and n is an integer of at least 2, for example at least 5, for example, at least 10, for example an integer of from about 2 to 200 or more, for example from about 2 to 20, for example from about 8 to 16, or from about 10 to 100, for example 30 to 80. Each R may be the same or different to other R moieties in the same conjugate.

It will be appreciated that the polymeric support structure may if desired comprise linker elements for coupling the modulators of Notch signalling to the polymeric support structure. In this case the conjugate may also be represented, for example, as:
POL(-L-R)ₙ
wherein POL is a polymeric support structure, each R independently represents a modulator of Notch signalling (each of which may be the same or different); each L independently represents either an optional linker moiety or residue (each of which may be the same or different) or a bond; and n is an integer as defined above.

Where numbers of modulators of Notch signalling present in a conjugate are indicated it will be appreciated that these may apply also to preparations, collections, or populations of conjugates, in which case the figure given may for example relate to the average number of modulators of Notch signalling per conjugate of the preparation, collection or population, suitably the mean number. For example, where it is stated that a conjugate has a number of modulators on Notch signalling in a given range, it will be appreciated that this can also be considered in terms of a preparation, collection or population of conjugates having an average (eg mean) number in the same range.

According to a further aspect of the invention there is provided a conjugate as defined above for use as a medicament.

According to a further aspect of the invention there is provided a conjugate as defined above for use in immunotherapy.

According to a further aspect of the invention there is provided the use of a conjugate as defined above in the manufacture of a medicament for modulation (increase or decrease) of an immune response.

According to a further aspect of the invention there is provided a method of modulating (increasing or decreasing) an immune response in a mammal by administering a conjugate as defined above.

According to a further aspect of the invention there is provided a method for preparing a conjugate as defined above by chemically combining a plurality of modulators of the Notch signalling pathway with a support structure, optionally by use of a linker.

Preferably the modulation of the immune system comprises immunotherapy.

Preferably the modulation of the immune system comprises modulation (increase or decrease) of T cell activity, suitably peripheral T cell activity.

Preferably the modulation of the immune system comprises modulation (increase or decrease) of the immune response to an antigen or antigenic determinant.

Alternatively or in addition at least one of the modulators of the Notch signalling pathway may comprise Notch or a fragment, derivative, homologue, analogue or allelic variant thereof or a polynucleotide encoding Notch or a fragment, derivative, homologue, analogue or allelic variant thereof.

Suitably at least one of the modulators of the Notch signalling pathway comprises a modulator of Notch signalling in the form of a protein or polypeptide consisting essentially of the following components:

i) a Notch ligand DSL domain;

ii) 1-5 and no more than 5 Notch ligand EGF domains;

iii) optionally all or part of a Notch ligand N-terminal domain; and

iv) optionally one or more heterologous amino acid sequences.

Suitably at least one of the modulators of the Notch signalling pathway comprises a modulator of Notch signalling in the form of a protein or polypeptide consisting essentially of the following components:

i) a Notch ligand DSL domain;

ii) 2-4 and no more than 4 Notch ligand EGF domains;

iii) optionally all or part of a Notch ligand N-terminal domain; and

iv) optionally one or more heterologous amino acid sequences.

Suitably at least one of the modulators of the Notch signalling pathway comprises a modulator of Notch signalling in the form of a protein or polypeptide consisting essentially of the following components:

i) a Notch ligand DSL domain;

ii) 2-3 and no more than 3 Notch ligand EGF domains;

iii) optionally all or part of a Notch ligand N-terminal domain; and

iv) optionally one or more heterologous amino acid sequences.

Suitably at least one of the modulators of the Notch signalling pathway comprises a modulator of Notch signalling in the form of a protein or polypeptide consisting essentially of the following components:

i) a Notch ligand DSL domain;

ii) 3 Notch ligand EGF domains;

iii) optionally all or part of a Notch ligand N-terminal domain; and

iv) optionally one or more heterologous amino acid sequences.

Suitably at least one of the modulators of the Notch signalling pathway comprises a modulator of Notch signalling in the form of a protein or polypeptide comprising:

i) a Notch ligand DSL domain;

ii) 1-5 Notch ligand EGF domains;

iii) optionally all or part of a Notch ligand N-terminal domain; and

iv) optionally one or more heterologous amino acid sequences.

Suitably at least one of the modulators of the Notch signalling pathway comprises a modulator of Notch signalling in the form of a protein or polypeptide comprising:

i) a Notch ligand DSL domain;

ii) 2-8 Notch ligand EGF domains;

iii) optionally all or part of a Notch ligand N-terminal domain; and

iv) optionally one or more heterologous amino acid sequences.

Suitably at least one of the modulators of the Notch signalling pathway comprises a modulator of Notch signalling in the form of a protein or polypeptide comprising:

i) a Notch ligand DSL domain;

ii) 2-5 Notch ligand EGF domains;

iii) optionally all or part of a Notch ligand N-terminal domain; and

iv) optionally one or more heterologous amino acid sequences.

Suitably at least one of the modulators of the Notch signalling pathway comprises a modulator of Notch signalling in the form of a protein or polypeptide comprising:

i) a Notch ligand DSL domain;

ii) 3 Notch ligand EGF domains;

iii) optionally all or part of a Notch ligand N-terminal domain; and

iv) optionally one or more heterologous amino acid sequences.

Suitably the domains comprise Delta or Jagged DSL or EGF domains.

Suitably the domains comprise human Delta DSL or EGF domains.

Suitably at least one of the modulators of Notch signalling comprises a polypeptide which has at least 50% (suitably at least 70%, suitably at least 90%) amino acid sequence similarity or identity to the following sequence along the entire length of the latter:

| | |
|---|---|
| MGSRCALALAVLSALLCQVWSSGVFELKLQEFVNKK | (SEQ ID NO:41) |
| GLLGNRNCCRGGAGPPPCACRTFFRVCLKHYQASVS | |
| PEPPCTYGSAVTPVLGVDSFSLPDGGGADSAFSNPI | |
| RFPFGFTWPGTFSLIIEALHTDSPDDLATENPERLI | |
| SRLATQRHLTVGEEWSQDLHSSGRTDLKYSYRFVCD | |
| EHYYGEGCSVFCRPRDDAFGHFTCGERGEKVCNPGW | |
| KGPYCTEPICLPGCDEQHGFCDKPGECKCRVGWQGR | |
| YCDECIRYPGCLHGTCQQPWQCNCQEGWGGLFCNQD | |
| LNYCTHHKPCKNGATCTNTGQGSYTCSCRPGYTGAT | |
| CELGIDEC | |

In one embodiment at least one of the modulators of the Notch signalling pathway may comprise an antibody, antibody fragment or antibody derivative.

According to a further aspect of the invention there is provided a method for preparing a conjugate as described above by combining a plurality of modulators of the Notch signalling pathway with a polymeric support structure.

According to a further aspect of the invention there is provided a method for preparing a conjugate as described above by:

i) providing a polymeric support structure; and

ii) reacting the polymeric support structure with a plurality of modulators of Notch signalling.

According to a further aspect of the invention there is provided a method for preparing a conjugate as described above by:

i) providing a polymeric support structure;

ii) activating the polymeric support structure; and

iii) reacting the activated polymeric support structure with a plurality of modulators of Notch signalling.

According to a further aspect of the invention there is provided a product comprising:

i) a conjugate as described above; and

ii) an antigen or antigenic determinant or a polynucleotide coding for an antigen or antigenic determinant;

as a combined preparation for simultaneous, contemporaneous, separate or sequential use for modulation of the immune system.

According to a further aspect of the invention there is provided a product as described above wherein the antigen or antigenic determinant is an autoantigen or antigenic determinant thereof or a polynucleotide coding for an autoantigen or antigenic determinant thereof.

In one such embodiment the antigen or antigenic determinant may be an allergen or antigenic determinant thereof or a polynucleotide coding for an allergen or antigenic determinant thereof.

In another such ambodiment the antigen or antigenic determinant may be a transplant antigen or antigenic determinant thereof or a polynucleotide coding for a transplant antigen or antigenic determinant thereof.

In another embodiment the antigen or antigenic determinant may be a tumour antigen or antigenic determinant thereof or a polynucleotide coding for a tumour antigen or antigenic determinant thereof.

In another embodiment the antigen or antigenic determinant may be a pathogen antigen or antigenic determinant thereof or a polynucleotide coding for a pathogen antigen or antigenic determinant thereof

According to a further aspect of the invention there is provided a pathogen vaccine composition comprising:

i) a conjugate as described above; and

ii) a pathogen antigen or antigenic determinant thereof or a polynucleotide coding for a pathogen antigen or antigenic determinant thereof.

According to a further aspect of the invention there is provided a cancer vaccine composition comprising:

i) a conjugate as described above; and

ii) a cancer antigen or antigenic determinant thereof or a polynucleotide coding for a cancer antigen or antigenic determinant thereof.

According to a further aspect of the invention there is provided the use of a conjugate as described above for the manufacture of a medicament for modulation of expression of a cytokine selected from IL-10, IL-5, IL-2, TNF-alpha, IFN-gamma or IL-13.

According to a further aspect of the invention there is provided the use of a conjugate as described above for the manufacture of a medicament for increase of IL-10 expression.

According to a further aspect of the invention there is provided the use of a conjugate as described above for the manufacture of a medicament for decrease of expression of a cytokine selected from IL-2, IL-5, TNF-alpha, IFN-gamma or IL-13.

According to a further aspect of the invention there is provided the use of a conjugate as described above for the manufacture of a medicament for generating an immune modulatory cytokine profile with increased IL-10 expression and reduced IL-5 expression.

According to a further aspect of the invention there is provided the use of a conjugate as described above for the manufacture of a medicament for generating an immune modulatory cytokine profile with increased IL-10 expression and reduced IL-2, IFN-gamma, IL-5, IL-13 and TNF-alpha expression.

According to a further aspect of the invention there is provided the use of a conjugate as described above pharmaceutical composition comprising a conjugate as described above.

According to a further aspect of the invention there is provided a pharmaceutical composition comprising a conjugate as described aboveand a pharmaceutically acceptable carrier.

The term “enhanced biological or therapeutic effects” as used herein includes, for example, increased affinity, increased potency, increased efficacy, decreased toxicity, improved duration of activity or action, decreased side effects, improved bioavailability, improved pharmacokinetics, improved activity spectrum, and the like.

The term “which consists essentially of” or “consisting essentially of” as used herein means that the construct includes the sequences and domains identified but is substantially free of other sequences or domains, and in particular is substantially free of any other Notch or Notch ligand sequences or domains.

For avoidance of doubt the term “comprising” means that any additional feature or component may be present.

The terms “modulate”, “modulation” and “modulating” etc. include both increasing and decreasing the relevant effect or signalling.

### BRIEF DESCRIPTION OF THE DRAWINGS

Various preferred features and embodiments of the present invention will now be described in more detail by way of non-limiting example and with reference to the accompanying Figures, in which:

FIG. 1 shows a schematic representation of the Notch signalling pathway;

FIG. 2 shows schematic representations of the Notch ligands Jagged and Delta;

FIG. 3 shows aligned amino acid sequences of DSL domains from various Drosophila and mammalian Notch ligands; (SEQ ID NOs:46-61)

FIGS. 4A-4C show amino acid sequences of human Delta-1 (4A; SEQ ID NO:62), Delta-3 (4B; SEQ ID NO:63) and Delta-4 (4C; SEQ ID NO:64);

FIGS. 5A and 5B show amino acid sequences of human Jagged-1 (5A; SEQ ID NO:65) and Jagged-2 (5B; SEQ ID NO:66);

FIG. 6 shows an amino acid sequence of human Notch1 (SEQ ID NO:67);

FIG. 7 shows an amino acid sequence of human Notch2 (SEQ ID NO:68);

FIG. 8 shows schematic representations of various Notch ligand fusion proteins which may be used as modulators of Notch signalling in the present invention;

FIG. 9 shows a small part of the structure of a dextran-maleimido-Notch ligand protein conjugate according to one particular embodiment of the invention. For simplicity only a small part of the structure is shown; it will be appreciated that the dextran backbone is typically very much longer than shown here (as indicated by “ . . . ”) and normally will be attached via a maleimido linker of the type shown to more than 3, suitably more than 20 or about 50 or more Notch ligands in a similar manner to that shown here for one such protein/polypeptide. The linker may also be attached to the dextran at other carbon atoms in the glucose (monomer) ring than that shown;

FIG. 10 shows a schematic representation of the construction of a dextran conjugate according to one embodiment of the invention. Again, for simplicity, only a small part of the structure is shown; it will be appreciated that the dextran backbone is typically very much longer than shown here (as indicated by “ . . . ”) and normally will be attached to more than 10, suitably more than 20 or about 50 or more Notch ligand protein/polypeptide in a generally similar manner to that shown here;

FIG. 11 shows results from Example 4;

FIGS. 12 and 13 show results from Example 5(i);

FIGS. 14 to 18 show results from Example 6;

FIGS. 19 to 21 show results from Example 7; and

FIGS. 22 and 23 show results from Example 8.

### DETAILED DESCRIPTION

Support Structures

Preferably the support structure used in the conjugate is a polymeric structure which is preferably a pharmaceutically acceptable polymer. Preferred polymers are water soluble polymers such as polyethylene glycol, ethylene glycol/propylene glycol copolymers, carboxymethylcellulose, dextran, polyvinyl alcohol and the like. Other suitable polymers include, for example, polyethylene glycol propionaldehyde, monomethoxy-polyethylene glycol, polyvinyl pyrrolidone (PVP), poly-1,3-dioxolane, poly-1,3,6-trioxane, ethylene/maleic anhydride copolymer, (either homopolymers or random copolymers), poly(n-vinyl pyrrolidone)polyethylene glycol, polypropylene glycol homopolymers (PPG) and other polyalkylene oxides, polypropylene oxide/ethylene oxide copolymers, polyoxyethylated polyols (POG) (e.g., glycerol) and other polyoxyethylated polyols, polyoxyethylated sorbitol, polyoxyethylated glucose, colonic acids or other carbohydrate polymers, Ficoll or dextran and mixtures thereof. It will be appreciated that polymers may also be used in activated, functionalised or derivatised forms

Modulators of Notch signalling may be attached to the support structure at random positions within the molecule, or at predetermined positions within the molecule and may be attached to one, two, three or more chemical moieties.

Polymers may be either homopolymers or copolymers, eg random copolymers and may be either straight or branched.

In certain embodiments polymers may be used in the form of hydrogels. The term “hydrogel” includes a solution of polymers, sometimes referred to as a sol, converted into gel state for example by small ions or polymers of the opposite charge or by chemical crosslinking.

Suitable polymers also include pharmaceutically acceptable dendrimers, including “Starburst”™ dendrimers available for example, from the Dow Chemical Company (Midland, Mich., US). For example, such dendrimers are described in U.S. Pat. No. 6,177,414 (Dow Chemical Company). As described therein, starburst polymers exhibit molecular architecture characterized by regular dendritic branching with radial symmetry. These radially symmetrical molecules are referred to as possessing “starburst topology”. These polymers are made in a manner which can provide concentric dendritic tiers around an initiator core. The starburst topology is achieved by the ordered assembly of organic repeating units in concentric, dendritic tiers around an initiator core; this is accomplished by introducing multiplicity and self-replication (within each tier) in a geometrically progressive fashion through a number of molecular generations. The resulting highly functionalized molecules have been termed “dendrimers” with reference to their branched (tree-like) structure as well as their oligomeric nature.

Suitably the polymer may be a polysaccharide polymer, such as a glucan, for example a dextran or a dextran derivative such as amino-dextran.

A polymer where used may be of any molecular weight, and may be branched or unbranched. Where polyethylene glycol is used, the preferred molecular weight is between about 1 kDa and about 500 kDa (the term “about” indicating for example that in preparations of polyethylene glycol, some molecules will weigh more, some less, than the stated molecular weight) for ease of handling and manufacturing. Other sizes may be used, depending on the desired therapeutic profile (e.g., the effects, if any on biological activity, the ease of handling, the degree or lack of antigenicity and other known effects of the polyethylene glycol to a therapeutic protein or analog). For example, the polymer may have an average molecular weight of about 200, 500, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000, 25,000, 30,000, 35,000, 40,000, 50,000, 55,000, 60,000, 65,000, 70,000, 75,000, 80,000, 85,000, 90,000, 95,000, or 100,000 Da.

Where carbohydrate polmers such as dextrans are used these may have an average molecular weight from about 1 kDa to about 10,000 kDa, for example from about 10 kDa to about 5,000 kDa, for example from about 100 kDa to about 3,000 kDa, suitably from about 100 kDa to about 1,000 kDa, for example about 500 kDa.

Where molecular weight figures are given for polymers, it will be appreciated that these apply also to preparations, collections, or populations of polymers/conjugates, in which case the figure given may for example relate to the average molecular weight of the preparation, collection or population, suitably the mean molecular weight. For example, where it is stated that a polymer molecule has a molecular weight in a given range, it will be appreciated that this can also be considered in terms of a preparation, collection or population of polymer molecules having a mean molecular weight in the same range.

A polymer where used may, if desired, have a branched structure. For example, branched polyethylene glycols are described, for example, in U.S. Pat. No. 5,643,575; Morpurgo et al., Appl. Biochem. Biotechnol. 56:59-72 (1996); Vorobjev et al., Nucleosides Nucleotides 18:2745-2750 (1999); and Caliceti et al., Bioconjug. Chem. 10:638-646 (1999), the disclosures of each of which are incorporated herein by reference.

Where the modulator of Notch signalling is a protein, the protein should preferably be attached to the support structure with consideration of effects on functional or antigenic domains of the protein. There are a number of attachment methods available to those skilled in the art, e.g., EP 0 401 384, herein incorporated by reference (coupling PEG to G-CSF), see also Malik et al., Exp. Hematol. 20:1028-1035 (1992) (reporting pegylation of GM-CSF using tresyl chloride). For example, polymers such as polyethylene glycol may be covalently bound through amino acid residues via a reactive group, such as a free amino or carboxyl group. Reactive groups are those to which an activated polymer such as polyethylene glycol molecule may be bound. The amino acid residues having a free amino group may include, for example, lysine residues and N-terminal amino acid residues; those having a free carboxyl group may include aspartic acid residues, glutamic acid residues, and the C-terminal amino acid residue. Sulfhydryl groups from cysteine residues may also be used as a reactive group for attaching polymers such as polyethylene glycol molecules. For example, attachment may be at an amino group, such as attachment at the N-terminus or a lysine group, or at a cysteine group, for example a C-terminal cysteine group.

Polymers such as polyethylene glycol may be attached to proteins and polypeptides via linkage to any of a number of amino acid residues of the protein or polypeptide. For example, polymers such as polyethylene glycol can be linked to a protein via covalent bonds to lysine, histidine, aspartic acid, glutamic acid, or cysteine residues. One or more reaction chemistries may be employed to attach polymers such as polyethylene glycol to specific amino acid residues (e.g., lysine, histidine, aspartic acid, glutamic acid, or cysteine) of the protein or to more than one type of amino acid residue (e.g., lysine, histidine, aspartic acid, glutamic acid, cysteine and combinations thereof) of the protein.

In some instances, it may be desirable to have proteins attached to the support structure through their N-termini. For example, using polyethylene glycol as an illustration, one may select from a variety of polyethylene glycol molecules (by molecular weight, branching, etc.), the proportion of polyethylene glycol molecules to protein (or peptide) molecules in the reaction mix, the type of pegylation reaction to be performed, and the method of obtaining the selected N-terminally pegylated protein. The method of obtaining the N-terminally pegylated preparation (i.e., separating this moiety from other monopegylated moieties if necessary) may be by purification of the N-terminally pegylated material from a population of pegylated protein molecules. Selective proteins chemically modified at the N-terminus modification may be accomplished by reductive alkylation which exploits differential reactivity of different types of primary amino groups (lysine versus the N-terminal) available for derivatization in a particular protein. Under the appropriate reaction conditions, substantially selective derivatization of the protein at the N-terminus with a carbonyl group containing polymer may be achieved.

Suitably, to provide an optimal orientation, proteins, polypeptides or peptides may be attached to the support structure through a suitably provided terminal residue, for example an C-terminal residue such as a terminal lysine, histidine, aspartic acid, glutamic acid or cysteine residue, which may be readily created or exposed by genetic manipulation techniques if not already present in the protein or peptide to be attached. Attachment at a terminal residue, or at a point close to the protein/peptide terminus (preferably C-terminus), typically provides better presentation of ligands for binding to and/or activation of Notch receptors.

For example, in a preferred form of the invention a multiplicity of protein/peptide modulators of Notch signalling (such as Notch ligand constructs comprising a DSL domain and 1-5, e.g. 3 EGF domains) are attached to a water-soluble polymeric support such as a polysaccharide, e.g. a dextran, by C-terminal residues (e.g. cysteine, lysine, histidine, glutamic or aspartic acid) via a linker such as sulfosuccinimidyl 4-[N-maleimidomethyl]-cyclohexane-1-carboxylate (sulfo-SMCC) or the like.

Carbohydrate/Polysaccharide Conjugates

In one embodiment of the invention the support structure may be a carbohydrate polymer, preferably a polysaccharide polymer. Preferably such a polysaccharide is water-soluble.

As is well-known, polysaccharides are generally made up of a number of monosaccharide units typically joined by glycosidic bonds, such as 1-4 or 1-6 linkages. Suitably the monosaccharide units may be, for example, aldoses (which may for example be trioses, tetroses such as erythrose or threose; pentoses such as ribose, arabinose, xylose or lyxose; hexoses such as allose, altrose, glucose, mannose, gulose, idose, galactose or tulose, or heptoses); or ketoses (which may for example be ketotrioses, ketotetroses such as erythulose; ketopentoses such as ribulose or xylulose; ketohexoses such as fructose, psicose, tagatose or sorbose, or ketoheptoses). Units may be in either D- or L-form, but the D form is generally preferred (eg D-glucose). Likewise, monosaccharide units may be in either alpha or beta forms, for example alpha-D-glucose. The monosaccharides in a polysaccharide may be substantially the same (ie to provide a homopolysaccharide) or combinations of units may be used (ie to provide a heteropolysaccharide). Tens, hunreds or thousands of monosaccharide units may be present in such a polymer, and branching will commonly be present.

Suitable carbohydrate polymers include for example, glucans such as dextrans including aminodextrans and carboxymethyl-dextrans, heparins, celluloses (and derivatives thereof such as methylcellulose, carboxymethylcellulose, ethylcellulose, hydroxyethylcellulose, carboxyethylcellulose and hydroxypropylcellulose), chitosan and hydrolysates of chitosan, starches (and derivatives thereof such as hydroxyethyl-starches and hydroxy propyl-starches), glycogens, heparins, alginates, agaroses and derivatives and activated versions thereof, guar gums, pullulans, inulins, xanthan gums, carrageenans, pectins and alginic acid hydrolysates and derivatives and activated versions thereof.

For example, a review of dextran conjugation is provided by Mehvar, Journal of Controlled Release Vol 69 (2000) pages 1-25.

As noted above, derivatives of such polymers (“derivatised polymers”) may also be used in the present invention. Such derivatised polymers may typically for example result from activation processes as described below.

Activation of Polymers

If desired, to conjugate modulators of Notch signalling (eg proteins, polypeptides or peptides, or mimetics thereof such as “small molecules”) to a polymer support material a number of groups on the polymer may be converted into more reactive functional groups which facilitate conjugation. This process is frequently referred to as “activation” and the product is called an “activated” or “functionalized” polymer.

In particular, if a polymeric molecule to be used as a support is not active (or is not considered sufficiently active) on its own it should preferably be activated by the use of a suitable technique.

Modulators of Notch signalling are preferably covalently attached to a polymer or activated polymer (either directly or via a linker) using chemical techniques. Reaction chemistries resulting in such linkages are well known in the art and may for example involve the use of complementary functional groups (eg on the linker, polymer and/or modulator of Notch signalling) for example as shown below:

| First Reactive | Second Reactive | |
|---|---|---|
| Group | Group | Linkage |
| carboxyl | amine | amide |
| sulfonyl halide | amine | sulfonamide |
| hydroxyl | alkyl/aryl halide | ether |
| hydroxyl | isocyanate | urethane |
| amine | epoxide | beta-hydroxyamine |
| amine | alkyl/aryl halide | alkylamine |
| hydroxyl | carboxyl | ester |
| amine | aldehyde | amide/amine |
| thiol/sulfhydryl | maleimide | — |
| amine | succinimide | — |

As described, for example, in U.S. Pat. No. 6,303,752 (Novozymes), methods and chemistry for activation of polymeric molecules as well as for conjugation of proteins, polypeptides and peptides are well described in the literature. For example, commonly used methods for activation of polymers include activation of functional groups with cyanogen bromide, periodate, glutaraldehyde, biepoxides, epichlorohydrin, divinylsulfone, carbodiimide, sulfonyl halides, trichlorotriazine etc. (see R. F. Taylor, (1991), “Protein immobilisation. Fundamental and applications”, Marcel Dekker, N.Y.; S. S. Wong, (1992), “Chemistry of Protein Conjugation and Crosslinking”, CRC Press, Boca Raton; G. T. Hermanson et al., (1993), “Immobilized Affinity Ligand Techniques”, Academic Press, N.Y. and Hermanson (1995) “Bioconjugate Techniques”, Academic Press, N. Y.). Some of these methods concern activation of insoluble polymers but are also applicable to activation of soluble polymers e.g. periodate, trichlorotriazine, sulfonylhalides, divinylsulfone, carbodiimide etc. The functional groups on the polymer and the chosen attachment group on the protein must be considered in choosing the activation and conjugation chemistry which may typically comprise i) activation of polymer, ii) conjugation, and iii) if required, blocking of residual active groups.

For example, coupling polymeric molecules to the free acid groups of polypeptides may be performed for example with the aid of diimide and for example amino-PEG or hydrazino-PEG (Pollak et al., (1976), J. Amr. Chem. Soc., 98, 289-291) or diazoacetate/amide (Wong et al., (1992), “Chemistry of Protein Conjugation and Crosslinking”, CRC Press).

Coupling to free sulfhydryl groups (such as a cysteine residue in a protein or polypeptide) can be achieved for example with groups like maleimido or ortho-pyridyl disulfide. Also vinylsulfone (U.S. Pat. No. 5,414,135, (1995), Snow et al.) has a preference for sulfhydryl groups.

Accessible arginine residues in a polypeptide chain may suitably be targeted by groups comprising two vicinal carbonyl groups.

Techniques involving coupling polymers such as electrophilically activated PEGs to the amino groups of reidues such as lysines may also be useful. Many of the usual leaving groups for alcohols give rise to an amine linkage. For instance, alkyl sulfonates, such as tresylates (Nilsson et al., (1984), Methods in Enzymology vol. 104, Jacoby, W. B., Ed., Academic Press: Orlando, p. 56-66; Nilsson et al., (1987), Methods in Enzymology vol. 135; Mosbach, K., Ed.; Academic Press: Orlando, pp. 65-79; Scouten et al., (1987), Methods in Enzymology vol. 135, Mosbach, K., Ed., Academic Press: Orlando, 1987; pp 79-84; Crossland et al., (1971), J. Amr. Chem. Soc. 1971, 93, pp. 4217-4219), mesylates (Harris, (1985), supra; Harris et al., (1984), J. Polym. Sci. Polym. Chem. Ed. 22, pp 341-352), aryl sulfonates like tosylates, and para-nitrobenzene sulfonates can be used.

Organic sulfonyl chlorides, e.g. tresyl chloride, effectively convert hydroxy groups in a number of polymers, e.g. PEG, into good leaving groups (sulfonates) that, when reacted with nucleophiles like amino groups in proteins or polypeptides allow stable linkages to be formed between polymer and polypeptide. In addition to high conjugation yields, the reaction conditions are in general mild (neutral or slightly alkaline pH, to avoid denaturation and little or no disruption of activity). Epoxides may also be used for creating amine bonds.

Converting PEG into a chloroformate with phosgene may facilitate carbamate linkages to lysines. The many variations include substituting the chlorine with N-hydroxy succinimide (U.S. Pat. No. 5,122,614, (1992); Zalipsky et al., (1992), Biotechnol. Appl. Biochem., 15, p. 100-114; Monfardini et al., (1995), Bioconjugate Chem., 6, 62-69, with imidazole (Allen et al., (1991), Carbohydr. Res., 213, pp 309-319), with para-nitrophenol, DMAP (EP 632 082 A1, (1993), Looze, Y.) etc. The derivatives are typically made for example by reacting the chloroformate with the desired leaving group. All these groups give rise to carbamate linkages to the peptide. Alternatively, isocyanates and isothiocyanates may be employed yielding ureas and thioureas, respectively.

In a further coupling technique, urethane (carbamate) linkages may be formed between an amino acid amino group (eg lysine, histidine, N-terminal residue), and an activated polymer. Suitably, such a urethane linkage is formed using a terminal oxycarbonyl-oxy-N-dicarboximide group such as a succinimidyl carbonate group. Alternative activating groups include N-succinimide, N-phthalimide, N-glutarimide, N-tetrahydrophthalimide and N-norborene-2,3-dicarboxide. These urethane-forming groups are described for example in U.S. Pat. No. 5,122,614, the disclosure of which is hereby incorporated by reference. This patent also discloses the formation of N-succinimide carbonate derivatives of polyalkylene oxides including polyethylene glycols which are also capable of forming urethane linkages with amino group targets (eg lysine).

Suitable starting materials and reagents for preparing the conjugates of the present invention are either available from commercial suppliers such as Aldrich Chemical Co., (Milwaukee, Wis., USA), Bachem (Torrance, Calif., USA), Emka-Chemie, or Sigma (St. Louis, Mo., USA), Pierce Chemical Company (Rockford, Ill., US), Molecular Probes Inc (Eugene, Oreg., US) or Amersham Pharmacia (Little Chalfont, UK and Piscataway, N.J., US); or are prepared by methods known to those skilled in the art following procedures set forth in references such as Fieser and Fieser's Reagents for Organic Synthesis, Volumes 1-15 (John Wiley and Sons, 1991); Rodd's Chemistry of Carbon Compounds, Volumes 1-5 and Supplementals (Elsevier Science Publishers, 1989), Organic Reactions, Volumes 1-40 (John Wiley and Sons, 1991), March's Advanced Organic Chemistry, (John Wiley and Sons, 4th Edition), and Larock's Comprehensive Organic Transformations (VCH Publishers Inc., 1989).

Additionally, it will be appreciated that conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in T. W. Greene and G. M. Wuts, Protecting Groups in Organic Synthesis, Second Edition, Wiley, N.Y., 1991, and references cited therein.

Preferably a linker reagent for use in the present invention may be a bifunctional reagent with a group for reacting with a modulator of Notch signalling (for example for reacting with a protein or polypeptide modulator of Notch signalling) and a group for reacting with a polymer support structure. After reaction the linker reagent may typically remain in the resulting conjugate as a linker reagent residue (which may also be termed, for example, a “linker”).

A wide range of linker reagents are available for example from the Pierce Chemical Company, Rockford, Ill., USA., (see for example Pierce Chemical Company, Cross-linking Technical Section, Pierce Life Science and Analytical Research products Catalog and Handbook, 1994), for example as follows:
p-azidobenzoyl hydrazide (ABH)
3-([2-aminoethyl]dithio)-propionic acid (AEDP)
N-alpha-maleimidoacetoxy)-succinimide ester (AMAS)
N-5-azido-2-nitrobenzyloxysuccinimide ANB-NOS)
N-(4-[p-azidosalicylamido]-butyl)-3′(2′pyridylthio)-propionamide (APDP)
p-azidophenyl glyoxal monohydrate (APG)
4-(p-azidosalicylamido)-butylamine (ASBA)
Bis(beta-[4-azidosalicylamido]-ethyl)disulfide (BASED)
1,4-Bis-Maleimidobutane (BMB)
1,4-Bis-Maleimidyl-2,3-dihydroxybutane (BMDB)
1,6-Bis-maleimidohexane (BMH)
Bis-Maleimidoethane (BMOE)
N-beta-maleimidopropionic acid (BMPA)
1,8-Bis-maleimidotriethylene glycol (BM[PEO]3)
1,11-Bis-maleimidotetraethylene glycol BM[PEO]4
N-(beta-maleimidopropionic acid)hydrazide.TFA (BMPH)
N-(beta-maleimidipropyloxy)succinimide ester (BMPS)
Bis(2-[succinimidooxy-carbonyloxy]ethyl)sulfone (BSOCOES)
Bis(sulfosuccinimidyl)-suberate (BS³)
1,5-difluoro-2,4-dinitrobenzene (DFDNB)
Dimethyladipimidiate (DMA)
Dimethylsuberimidate (DMS)
1,4-Di-(3′-[2′pyridylthio]-propionamido)butane (DPDPB)
Disuccinimidyl glutarate (DSG)
Dithiobis(succinimidylpropionate) (DSP)
Disuccinimidyl suberate (DSS)
Disuccinimidyl tartarate (DST)
Dimethyl 3,3′-dithiobis-propionimidate (DTBP)
Dithio-bis-maleimidoethane (DTME)
3,3′-dithiobis(sulfosuccinimidylpropionate) (DTSSP)
Ethylene glycol bis(succinimidylsuccinate) (EGS)
N-epsilon-maleimidocaproic acid (ECMA)
N-epsilon-(maleimidocaprolyloxy)succinimide ester (EMCS)
N-gamma-maleimidobutyryloxy-succinimide ester (GMBS)
1,6-hexane-bis-vinylsulfone (HBVS)
N-kappa-malaimidoundecanoic acid (KMUA)
Succinimidyl -4-(N-maleimido-methyl)cyclohexane-1-carboxy-(6-amido caproate) (LC-SMCC)
Succinimidyl 6-(3′-[2-pyridyl-dithio]propionamido)hexanoate (LC-SPDP)
m-maleimidobenzoyl-N-hydroxysuccinimide ester (MBS)
4-(N-maleimidomethyl)-cyclohexane-1-carboxyl-hydrazide (M2C2H)
3-maleimidophenyl boronic acid (MPBA)
4-(4-N-maleimidophenyl)-butyric acid hyrdrazide (MPBH)
Methyl N-succinimidyl adipate (MSA)
N-Hydroxysuccinimidyl-4-azidosalicylic acid (NHS-ASA)
3-(2-pyridylthio)-propionyl hydrazide (PDPH)
N-(p-maleimidophenyl)isocyanate (PMPI)
N-succinimidyl(4′azido-phenyl)1,3′-dithiopropionate (SADP)
Sulfosuccinimidyl-2-[7-azido-4-methylcoumarin-3-acetamido]ethyl-1,3′-dithiopropionate (SAED)
Sulfosuccinimidyl-2-(m-azido-o-nitrobenzamido)ethyl 1,3′-dithiopropionate (SAND)
N-succinimidyl 6-(4′-azido-2′-nitrophenylamino)hexanoate (SANPAH)
Sulfosuccinimidyl 2-(p-azidosalicylamido)ethyl 1,3-dithiopropionate (SASD)
N-succinimidyl S acetylthioacetate (SATA)
N-succinimidyl S-acetylthiopropionate (SATP)
Succinimidyl 3-(bromoacetamido)propionate (SBAP)
Sulfosuccinimidyl(perfluoroazidobenzamido)ethyl 1,3′-dithiopropionate (SFAD)
N-succinimidyl iodoacetate (SIA)
N-succinimidyl(4-iodoacetyl)aminobenzoate (SIAB)
Succinimidyl 4-(N-maleimido-methyl)cyclohexane-1-carboxylate (SMCC)
Succinimidyl 4-(p-maleimidophenyl)butyrate (SMPB)
Succinimidyl-6-(beta-maleimido-propionamido)hexanoate (SMPH)
4-succinimidyloxy-carbonyl-methyl-alpha-(2-pyridylthio)toluene (SMPT)
Succinimidyl-(4-psoralen-8-yloxy)butyrate (SPB)
N-succinimidyl 3-(2-pyridylthio)propionate (SPDP)
Bis(2-[sulfosuccinimidooxycarbonyloxy]ethyl)sulfone (Sulfo-BSOCOES)
Sulfodisuccinimidyl tartarate (Sulfo-DST)
Ethylene glycol bis(sulfo-succinimidyl)succcinate (Sulfo-EGS)
N-(epsilon-maleimidocaproyloxy)sulfosuccinimide ester (Sulfo-EMCS)
N-gamma-maleimidobutryloxy-sulfosuccinimide ester (Sulfo-GMBS)
N-hydroxysulfosuccinimidyl-4-azidobenzoate (Sulfo-HSAB)
N-(kappa-maleimidoundecanoyloxy)-sulfosuccinimide ester (Sulfo-KMUS)
Sulfosuccinimidyl 6-(3-[2-pyridyldithio]-propionamido)hexanoate (Sulfo-LC-SPDP)
m-maleimidobenzoyl-N-hydroxysulfosuccinimide ester (Sulfo-MBS)
Sulfosuccinimidyl(4-azido-salicylamido)hexanoate (Sulfo-NHS-LC-ASA)
Sulfosuccinimidyl(4-azidophenyldithio)propionate (Sulfo-SADP)
Sulfosuccinimidyl 6-(4′-azido-2′-nitrophenylamino)-hexanoate (sulfo-SANPAH)
Sulfo-NHS-2-(6-[biotinamido]-2-(p-azidobenzamido)-hexanoamido)ethyl-1,3′-Dithiopropionate (Sulfo-BED; trifunctional)
Sulfosuccinimidyl(4-iodo-acetyl)aminobenzoate (Sulfo-SIAB)
Sulfosuccinimidyl 4-(N-maleimidomethyl)cyclohexane-1-carboxylate (Sulfo-SMCC)
Sulfosuccinimidyl 4-(p-maleimidophenyl)butyrate (Sulfo-SMPB)
Sulfosuccinimidyl 6-(alpha-mathyl-alpha-[2-pyridyldithio]-toluamido)hexanoate (Sulfo-LC-SMPT)
N-succinimidyl-(4-vinylsulfonyl) benzoate (SVSB)
Tris-(2-maleimidoethyl) amine (TMEA; trifunctional)
Tris-(succinimidyl amino-triacetate (TSAT; trifunctional).

Suitably a linker used will be a bifunctional reagent, such as a heterobifunctional reagent (although it will be appreciated that homobifunctional reagents may also be used). Trifunctional and higher reagents may also be used if desired.

Suitably the modulators of Notch signalling are presented on the polymer in an orientation suitable for binding to and/or activation of a Notch receptor.

PEG Conjugates

As noted above, one preferred form of polymer for use in the present invention is polyethylene glycol (PEG) and derivatives thereof. In one form PEG may, for example, be a linear polymer terminated at each end with hydroxyl groups (as described, for example, in U.S. Pat. No. 6,362,254), for example:
HO—CH₂CH₂—O—(CH₂CH₂O)ₙ—CH₂CH₂—OH

This polymer can be represented in brief form as HO-PEG-OH where the -PEG- symbol represents the following structural unit:
—CH₂CH₂O—(CH₂CH₂O)ₙ—CH₂CH₂—

In typical form n is an integer of from about 10 to about 2000.

PEG is commonly used as methoxy PEG-OH, or mPEG in brief, in which one terminus is the relatively inert methoxy group, while the other terminus is a hydroxyl group that is subject to ready chemical modification.
CH₃O—(CH₂CH₂O)ₙ—CH₂CH₂—OH (mPEG)

PEG is also commonly used in branched forms that can be prepared by addition of ethylene oxide to various polyols, such as glycerol, pentaerythritol and sorbitol. For example, the four-arm, branched PEG prepared from pentaerythritol is shown below:
C(CH₂—OH)₄+nC₂H₄O→C[CH₂—O—(CH₂CH₂O)ₙ—CH₂CH₂—OH]₄
(wherein n is an integer of from about 10 to about 2000)

The branched PEGs can be represented in general form as R(-PEG-OH)ₙ in which R represents the central “core” molecule, such as glycerol or pentaerythritol, and n represents the number of “arms”.

Branched PEGs can also be prepared in which two PEG “arms” are attached to a central linking moiety having a single functional group capable of joining to other molecules; e.g., Matsushima et al., (Chem. Lett., 773, 1980) have coupled two PEGs to a central cyanuric chloride moiety.

A typical branched chain (or “multi-arm”) PEG may for example have the following structure: 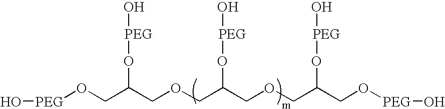
wherein each PEG element, which may be the same or different, is as defined above and m is an integer, typically from 0 to 100, for example 0 to 50, for example 4 to 20, for example 6 to 16.

PEG is a well known polymer having the properties of solubility in water and in many organic solvents, lack of toxicity, and lack of immunogenicity. One use of PEG is to covalently attach the polymer to insoluble molecules to make the resulting PEG-molecule “conjugate” soluble. For example, it has been shown that the water-insoluble drug paclitaxel, when coupled to PEG, becomes water-soluble. Greenwald, et al., J. Org. Chem., 60:331-336 (1995).

In related work, U.S. Pat. No. 4,179,337 (Davis et al) discloses that proteins coupled to PEG have enhanced blood circulation lifetime because of reduced rate of kidney clearance and reduced immunogenicity. These and other applications are also described in Biomedical and Biotechnical Applications of Polyethylene Glycol Chemistry, J. M. Harris, Ed., Plenum, N.Y. (1992), and Poly(ethylene glycol) Chemistry and Biological Applications, J. M. Harris and S. Zalipsky, Eds., ACS, Washington D.C. (1997), the texts of which are herein incorporated by reference.

Reaction of the modulator of Notch signalling with the support structure may be accomplished by many means. For example, where the modulator is a protein, polypeptide or peptide, polyethylene glycol may be attached to the protein polypeptide or peptide either directly or by an intervening linker. Linkerless systems for attaching polyethylene glycol to proteins are described in Delgado et al., Crit. Rev. Thera. Drug Carrier Sys. 9:249-304 (1992); Francis et al., Intern. J. of Hematol. 68:1-18 (1998); U.S. Pat. No. 4,002,531; U.S. Pat. No. 5,349,052; WO 95/06058; and WO 98/32466, the disclosures of each of which are incorporated herein by reference.

One system for attaching polyethylene glycol directly to amino acid residues of proteins without an intervening linker employs tresylated mPEG, which is produced by the modification of monmethoxy polyethylene glycol (mPEG) using tresylchloride (CISO₂ CH₂CF₃). Upon reaction of protein with tresylated mPEG, polyethylene glycol is directly attached to amine groups of the protein. Thus, the invention includes protein-polyethylene glycol conjugates produced by reacting proteins of the invention with a polyethylene glycol molecule having a 2,2,2-trifluoreothane sulphonyl group.

Polymers such as polyethylene glycol can also be attached to proteins using a number of different intervening linkers. For example, U.S. Pat. Publication No. 5,612,460, the text of which is incorporated herein by reference, discloses urethane linkers for connecting polyethylene glycol to proteins. Protein-polyethylene glycol conjugates wherein the polyethylene glycol is attached to the protein by a linker can also be produced by reaction of proteins with compounds such as mPEG-succinimidylsuccinate, mPEG activated with 1,1′-carbonyldiimidazole, MPEG-2,4,5-trichloropenylcarbonate, MPEG-p-nitrophenolcarbonate, and various MPEG-succinate derivatives. A number of additional polyethylene glycol derivatives and reaction chemistries for attaching polyethylene glycol to proteins are described in WO 98/32466, the text of which is incorporated herein by reference. One example of such an activated PEG derivative is the succinimidyl succinate “active ester”:
CH₃O-PEG-O₂C—CH₂CH₂—CO₂—NS
where NS has the structure:

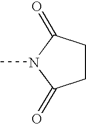

The succinimidyl active ester is a useful linker because it reacts rapidly with amino groups on proteins and other molecules to form an amide linkage (—CO—NH—). For example, U.S. Pat. Publication No. 4,179,337 (Davis et al) describes coupling of this derivative to proteins (represented as PRO-NH₂):
mPEG-O₂CCH₂CH₂CO₂NS+PRO-NH₂→mPEG-O₂C—CH₂CH₂—CONH-PRO

Other suitable “activated” PEGs include, for example PEG succinimidyl propionates and succinimidyl butanoates, N-hydroxysuccinimides, benzotriazole carbonates, propionaldehydes, maleimides and forked maleimides, biotins, vinyl derivatives and phospholipids,

Such PEGs and “activated” PEGs are available, for example, from Shearwater Corporation, Huntsville, Ala., USA.

Bifunctional PEGs with active groups at both ends of the linear polymer chain are also useful compounds when formation of a crosslinked insoluble network is desired. Many such bifunctional PEGs are known in the art. For example, U.S. Pat. No. 5,162,430 to Rhee, et al. discloses using such bifunctional PEGs to crosslink collagen.

Reactive PEGs have also been synthesized in which several active functional groups are placed along the backbone of the polymer. For example, lysine-PEG conjugates have been prepared in the art in which a number of activated groups are placed along the backbone of the polymer. Zalipsky et al. Bioconjugate Chemistry, 4:54-62 (1993).

Thus, in one embodiment a conjugate according to the present invention may, for example, have the following structure: 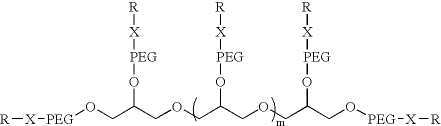
wherein each PEG element, which may be the same or different, is as defined above; each X, which may be the same or different, is independently a bond or a linker moiety as discussed above; m is an integer, suitably from 0 to 100, for example 0 to 50, for example 0 to 50, for example 4 to 20, for example 6 to 16, for example about 5 to about 10; and each R, which maybe the same or different, is independently a modulator of Notch signalling as defined herein or an end-group (optionally substituted) such as —OH, —CH₃ or —OCH₃.
General Techniques

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; D. M. J. Lilley and J. E. Dahlberg, 1992, Methods of Enzymology: DNA Structure Part A: Synthesis and Physical Analysis of DNA Methods in Enzymology, Academic Press; and J. E. Coligan, A. M. Kruisbeek, D. H. Margulies, E. M. Shevach and W. Strober (1992 and periodic supplements; Current Protocols in Immunology, John Wiley & Sons, New York, N.Y.). Each of these general texts is herein incorporated by reference.

For the avoidance of doubt, Drosophila and vertebrate names are used interchangeably and all homologues are included within the scope of the invention.

Modulators of Notch Signalling

The term “modulation of the Notch signalling pathway” as used herein refers to a change or alteration in the biological activity of the Notch signalling pathway or a target signalling pathway thereof. The term “modulator of the Notch signalling pathway” may refer to antagonists or inhibitors of Notch signalling, i.e. compounds which block, at least to some extent, the normal biological activity of the Notch signalling pathway. Conveniently such compounds may be referred to herein as inhibitors or antagonists. Alternatively, the term “modulator of the Notch signalling pathway” may refer to agonists of Notch signalling, i.e. compounds which stimulate or upregulate, at least to some extent, the normal biological activity of the Notch signalling pathway. Conveniently such compounds may be referred to as upregulators or agonists. Preferably the modulator is an agonist of Notch signalling, and preferably an agonist of the Notch receptor (eg an agonist of the Notch1, Notch2, Notch3 and/or Notch4 receptor, preferably being a human Notch receptor). Preferably such an agonist (“activator of Notch”) binds to and activates a Notch receptor, preferably including human Notch recpetors such as human Notch1, Notch2, Notch3 and/or Notch4. Binding to and/or activation of a Notch receptor may be assessed by a variety of techniques known in the art including in vitro binding assays and activity assays for example as described herein.

For example, whether any particular agent activates Notch signalling (e.g. is an activator of Notch or a Notch agonist) may be readily determined by use of any suitable assay, for example by use of a HES-1 reporter assay of the type described in Example 6 herein. Conversely, antagonist activity may be readily determined for example by monitoring any effect of the agent in reducing signalling by known Notch signalling agonists such as CHO-Delta cells, for example, as described in Example 6 herein (ie in a so-called “antagonist” assay).

In one embodiment, a modulator may be an organic compound or other chemical. For example, a modulator may be an organic compound comprising two or more hydrocarbyl groups. Here, the term “hydrocarbyl group” means a group comprising at least C and H and may optionally comprise one or more other suitable substituents. Examples of such substituents may include halo-, alkoxy-, nitro-, an alkyl group, a cyclic group etc. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain hetero atoms. Suitable hetero atoms will be apparent to those skilled in the art and include, for instance, sulphur, nitrogen and oxygen. The candidate modulator may comprise at least one cyclic group. The cyclic group may be a polycyclic group, such as a non-fused polycyclic group. For some applications, the agent comprises at least the one of said cyclic groups linked to another hydrocarbyl group.

In a preferred embodiment, the modulator will comprise an amino acid sequence or a chemical derivative thereof, or a combination thereof. The modulator may also be an antibody.

The term “antibody” includes intact molecules as well as fragments thereof, such as Fab, F(ab′)2, Fv and scFv which are capable of binding the epitopic determinant. These antibody fragments retain some ability to selectively bind with its antigen or receptor and include, for example:

(i) Fab, the fragment which contains a monovalent antigen-binding fragment of an antibody molecule can be produced by digestion of whole antibody with the enzyme papain to yield an intact light chain and a portion of one heavy chain;

(ii) Fab′, the fragment of an antibody molecule can be obtained by treating whole antibody with pepsin, followed by reduction, to yield an intact light chain and a portion of the heavy chain; two Fab′ fragments are obtained per antibody molecule;

(iii) F(ab′)₂, the fragment of the antibody that can be obtained by treating whole antibody with the enzyme pepsin without subsequent reduction; F(ab′)₂ is a dimer of two Fab′ fragments held together by two disulfide bonds;

(iv) scFv, including a genetically engineered fragment containing the variable region of a heavy and a light chain as a fused single chain molecule; and

(v) so-called “combibodies” constructed, for example by self-assembly from one constant and one variable region of each heavy and light chain.

(See for example, Harlow and Lane, Antibodies: A Laboratory Manual, Cold Spring Harbor Laboratory, New York (1988), which is incorporated herein by reference).

Modulators may be synthetic compounds or natural isolated compounds.

The conjugates of the present invention may if desired be provided in the form of pharmaceutically acceptable salts. For example, the conjugates may be capable of forming acid and/or base salts by virtue of the presence of amino and/or carboxyl groups or groups similar thereto.

Notch Signalling

As used herein, the expression “Notch signalling” is synonymous with the expression “the Notch signalling pathway” and refers to any one or more of the upstream or downstream events that result in, or from, (and including) activation of the Notch receptor.

Preferably, by “Notch signalling” we refer to any event directly upstream or downstream of Notch receptor activation or inhibition including activation or inhibition of Notch/Notch ligand interactions, upregulation or downregulation of Notch or Notch ligand expression or activity and activation or inhibition of Notch signalling transduction including, for example, proteolytic cleavage of Notch and upregulation or downregulation of the Ras-Jnk signalling pathway.

Thus, by “Notch signalling” we refer to the Notch signalling pathway as a signal tranducing pathway comprising elements which interact, genetically and/or molecularly, with the Notch receptor protein. For example, elements which interact with the Notch protein on both a molecular and genetic basis are, by way of example only, Delta, Serrate and Deltex. Elements which interact with the Notch protein genetically are, by way of example only, Mastermind, Hairless, Su(H) and Presenilin.

In a preferred aspect of the present invention, Notch signalling means signalling events taking place extracellularly or at the cell membrane. In a further aspect, it may also include signalling events taking place intracellularly, for example within the cell cytoplasm or within the cell nucleus.

In one form the modulator of the Notch signalling pathway may be a protein for Notch signalling transduction.

By a protein which is for Notch signalling transduction is meant a molecule which participates in signalling through Notch receptors including activation of Notch, the downstream events of the Notch signalling pathway, transcriptional regulation of downstream target genes and other non-transcriptional downstream events (e.g. post-translational modification of existing proteins). Preferably, the protein comprises a domain that allows activation of target genes of the Notch signalling pathway.

A very important component of the Notch signalling pathway is Notch receptor/Notch ligand interaction. In a preferred form of the invention the signalling may be specific signalling, meaning that the signal results substantially or at least predominantly from the Notch signalling pathway, and preferably from Notch/Notch ligand interaction, rather than any other significant interfering or competing cause such as cytokine signalling. Thus, in a preferred embodiment, the term “Notch signalling” as used herein excludes cytokine signalling. The Notch signalling pathway is described in more detail below.

Key targets for Notch-dependent transcriptional activation are genes of the Enhancer of split complex (E[spl]). Moreover these genes have been shown to be direct targets for binding by the Su(H) protein and to be transcriptionally activated in response to Notch signalling. By analogy with EBNA2, a viral coactivator protein that interacts with a mammalian Su(H) homologue CBF1 to convert it from a transcriptional repressor to a transcriptional activator, the Notch intracellular domain, perhaps in association with other proteins may combine with Su(H)/CBF1 to contribute an activation domain that allows Su(H)/CBF1 to activate the transcription of E(spl) as well as other target genes. It should also be noted that Su(H)/CBF1 is not required for all Notch-dependent decisions, indicating that Notch mediates some cell fate choices by associating with other DNA-binding transcription factors or be employing other mechanisms to transduce extracellular signals.

According to one aspect of the present invention the active agent may comprise a Notch protein or an analogue of a Notch protein.

As used herein the term “analogue of Notch” includes variants thereof which retain the signalling transduction ability of Notch. By “analogue” we include a protein which has Notch signalling transduction ability, but generally has a different evolutionary origin to Notch. Analogues of Notch include proteins from the Epstein Barr virus (EBV), such as EBNA2, BARF0 or LMP2A.

By a protein which is for Notch signalling activation we mean a molecule which is capable of activating Notch, the Notch signalling pathway or any one or more of the components of the Notch signalling pathway.

In a preferred embodiment, a modulator of Notch signalling for use in the present invention may comprise all or part of a Notch ligand, or a polynucleotide encoding a Notch ligand. Notch ligands of use in the present invention include endogenous (naturally occurring) Notch ligands which are typically capable of binding to a Notch receptor polypeptide present in the membrane of a variety of mammalian cells, for example hemapoietic stem cells and T-cells.

The term “Notch ligand” as used herein means an agent capable of interacting with a Notch receptor to cause a biological effect. The term as used herein therefore includes naturally occurring protein ligands (e.g. from Drosophila, verterbrates, mammals) such as Delta and Serrate/Jagged (e.g. mammalian ligands Delta1, Delta 3, Delta4, Jagged1 and Jagged2 and homologues) and their biologically active fragments as well as antibodies to the Notch receptor, as well as peptidomimetics, antibodies and small molecules which have corresponding biological effects to the natural ligands. Preferably the Notch ligand interacts with the Notch receptor by binding.

Particular examples of mammalian Notch ligands identified to date include the Delta family, for example Delta or Delta-like 1 (e.g. Genbank Accession No. AF003522—Homo sapiens); Delta-3 (e.g. Genbank Accession No. AF084576—Rattus norvegicus) and Delta-like 3 (Mus musculus) (e.g. Genbank Accession No. NM_{—}016941—Homo sapiens) and U.S. Pat. No. 6,121,045 (Millennium); Delta-4 (Genbank Accession Nos. AB043894 and AF 253468—Homo sapiens); and the Serrate family, for example Serrate-1 and Serrate-2 (WO97/01571, WO96/27610 and WO92/19734); Jagged-1 (Genbank Accession No. U73936—Homo sapiens) and Jagged-2 (Genbank Accession No. AF029778—Homo sapiens), and LAG-2. Homology between family members is extensive. Sequences of human Delta1, Delta3, Delta4, Jagged1 and Jagged2 are shown in the Figures hereto.

Notch Ligand Domains

Notch ligands comprise a number of distinctive domains. Some predicted/potential domain locations for various naturally occurring human Notch ligands (based on amino acid numbering in the precursor proteins) are shown below:

Human Delta 1

| Component | Amino acids | Proposed function/domain |
|---|---|---|
| SIGNAL | 1-17 | SIGNAL |
| CHAIN | 18-723 | DELTA-LIKE PROTEIN 1 |
| DOMAIN | 18-545 | EXTRACELLULAR |
| TRANSMEM | 546-568 | TRANSMEMBRANE |
| DOMAIN | 569-723 | CYTOPLASMIC |
| DOMAIN | 159-221 | DSL |
| DOMAIN | 226-254 | EGF-LIKE 1 |
| DOMAIN | 257-285 | EGF-LIKE 2 |
| DOMAIN | 292-325 | EGF-LIKE 3 |
| DOMAIN | 332-363 | EGF-LIKE 4 |
| DOMAIN | 370-402 | EGF-LIKE 5 |
| DOMAIN | 409-440 | EGF-LIKE 6 |
| DOMAIN | 447-478 | EGF-LIKE 7 |
| DOMAIN | 485-516 | EGF-LIKE 8 |

Human Delta 3

| Component | Amino acids | Proposed function/domain |
|---|---|---|
| DOMAIN | 158-248 | DSL |
| DOMAIN | 278-309 | EGF-LIKE 1 |
| DOMAIN | 316-350 | EGF-LIKE 2 |
| DOMAIN | 357-388 | EGF-LIKE 3 |
| DOMAIN | 395-426 | EGF-LIKE 4 |
| DOMAIN | 433-464 | EGF-LIKE 5 |

Human Delta 4

| Component | Amino acids | Proposed function/domain |
|---|---|---|
| SIGNAL | 1-26 | SIGNAL |
| CHAIN | 27-685 | DELTA-LIKE PROTEIN 4 |
| DOMAIN | 27-529 | EXTRACELLULAR |
| TRANSMEM | 530-550 | TRANSMEMBRANE |
| DOMAIN | 551-685 | CYTOPLASMIC |
| DOMAIN | 155-217 | DSL |
| DOMAIN | 218-251 | EGF-LIKE 1 |
| DOMAIN | 252-282 | EGF-LIKE 2 |
| DOMAIN | 284-322 | EGF-LIKE 3 |
| DOMAIN | 324-360 | EGF-LIKE 4 |
| DOMAIN | 362-400 | EGF-LIKE 5 |
| DOMAIN | 402-438 | EGF-LIKE 6 |
| DOMAIN | 440-476 | EGF-LIKE 7 |
| DOMAIN | 480-518 | EGF-LIKE 8 |

Human Jagged 1

| Component | Amino acids | Proposed function/domain |
|---|---|---|
| SIGNAL | 1-33 | SIGNAL |
| CHAIN | 34-1218 | JAGGED 1 |
| DOMAIN | 34-1067 | EXTRACELLULAR |
| TRANSMEM | 1068-1093 | TRANSMEMBRANE |
| DOMAIN | 1094-1218 | CYTOPLASMIC |
| DOMAIN | 167-229 | DSL |
| DOMAIN | 234-262 | EGF-LIKE 1 |
| DOMAIN | 265-293 | EGF-LIKE 2 |
| DOMAIN | 300-333 | EGF-LIKE 3 |
| DOMAIN | 340-371 | EGF-LIKE 4 |
| DOMAIN | 378-409 | EGF-LIKE 5 |
| DOMAIN | 416-447 | EGF-LIKE 6 |
| DOMAIN | 454-484 | EGF-LIKE 7 |
| DOMAIN | 491-522 | EGF-LIKE 8 |
| DOMAIN | 529-560 | EGF-LIKE 9 |
| DOMAIN | 595-626 | EGF-LIKE 10 |
| DOMAIN | 633-664 | EGF-LIKE 11 |
| DOMAIN | 671-702 | EGF-LIKE 12 |
| DOMAIN | 709-740 | EGF-LIKE 13 |
| DOMAIN | 748-779 | EGF-LIKE 14 |
| DOMAIN | 786-817 | EGF-LIKE 15 |
| DOMAIN | 824-855 | EGF-LIKE 16 |
| DOMAIN | 863-917 | VON WILLEBRAND |
| | | FACTOR C |

Human Jagged 2

| Component | Amino acids | Proposed function/domain |
|---|---|---|
| SIGNAL | 1-26 | SIGNAL |
| CHAIN | 27-1238 | JAGGED 2 |
| DOMAIN | 27-1080 | EXTRACELLULAR |
| TRANSMEM | 1081-1105 | TRANSMEMBRANE |
| DOMAIN | 1106-1238 | CYTOPLASMIC |
| DOMAIN | 178-240 | DSL |
| DOMAIN | 249-273 | EGF-LIKE 1 |
| DOMAIN | 276-304 | EGF-LIKE 2 |
| DOMAIN | 311-344 | EGF-LIKE 3 |
| DOMAIN | 351-382 | EGF-LIKE 4 |
| DOMAIN | 389-420 | EGF-LIKE 5 |
| DOMAIN | 427-458 | EGF-LIKE 6 |
| DOMAIN | 465-495 | EGF-LIKE 7 |
| DOMAIN | 502-533 | EGF-LIKE 8 |
| DOMAIN | 540-571 | EGF-LIKE 9 |
| DOMAIN | 602-633 | EGF-LIKE 10 |
| DOMAIN | 640-671 | EGF-LIKE 11 |
| DOMAIN | 678-709 | EGF-LIKE 12 |
| DOMAIN | 716-747 | EGF-LIKE 13 |
| DOMAIN | 755-786 | EGF-LIKE 14 |
| DOMAIN | 793-824 | EGF-LIKE 15 |
| DOMAIN | 831-862 | EGF-LIKE 16 |
| DOMAIN | 872-949 | VON WILLEBRAND |
| | | FACTOR C |

DSL Domain

A typical DSL domain may include most or all of the following consensus amino acid sequence (SEQ ID NO:1):

Cys Xaa Xaa Xaa Xaa Xaa Xaa Xaa Xaa Cys Xaa Xaa Xaa Cys Xaa Xaa
Xaa Xaa Xaa Xaa Xaa Xaa Xaa Xaa Xaa Cys Xaa Xaa Xaa Xaa Xaa Xaa
Xaa Cys Xaa Xaa Xaa Xaa Xaa Xaa Xaa Xaa Cys

Preferably the DSL domain may include most or all of the following consensus amino acid sequence (SEQ ID NO: 2):

Cys Xaa Xaa Xaa ARO ARO Xaa Xaa Xaa Cys Xaa Xaa Xaa Cys BAS NOP
BAS ACM ACM Xaa ARO NOP ARO Xaa Xaa Cys Xaa Xaa Xaa NOP Xaa Xaa
Xaa Cys Xaa Xaa NOP ARO Xaa NOP Xaa Xaa Cys

wherein:

ARO is an aromatic amino acid residue, such as tyrosine, phenylalanine, tryptophan or histidine;

NOP is a non-polar amino acid residue such as glycine, alanine, proline, leucine, isoleucine or valine;

BAS is a basic amino acid residue such as arginine or lysine; and

ACM is an acid or amide amino acid residue such as aspartic acid, glutamic acid, asparagine or glutamine.

Preferably the DSL domain may include most or all of the following consensus amino acid sequence (SEQ ID NO: 3):

Cys Xaa Xaa Xaa Tyr Tyr Xaa Xaa Xaa Cys Xaa Xaa Xaa Cys Arg Pro
Arg Asx Asp Xaa Phe Gly His Xaa Xaa Cys Xaa Xaa Xaa Gly Xaa Xaa
Xaa Cys Xaa Xaa Gly Trp Xaa Gly Xaa Xaa Cys

(wherein Xaa may be any amino acid and Asx is either aspartic acid or asparagine).

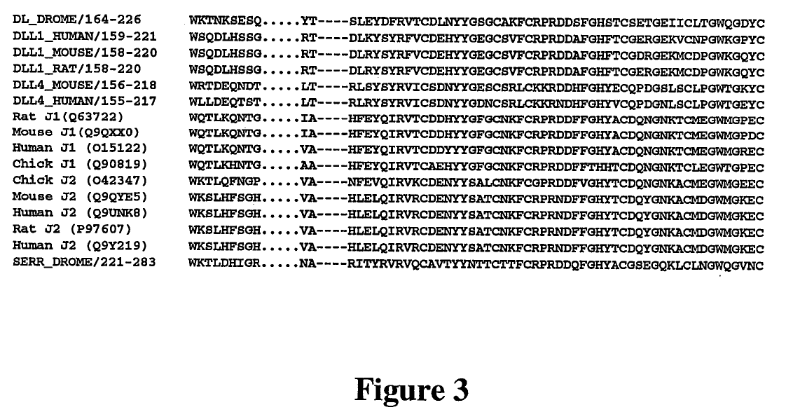

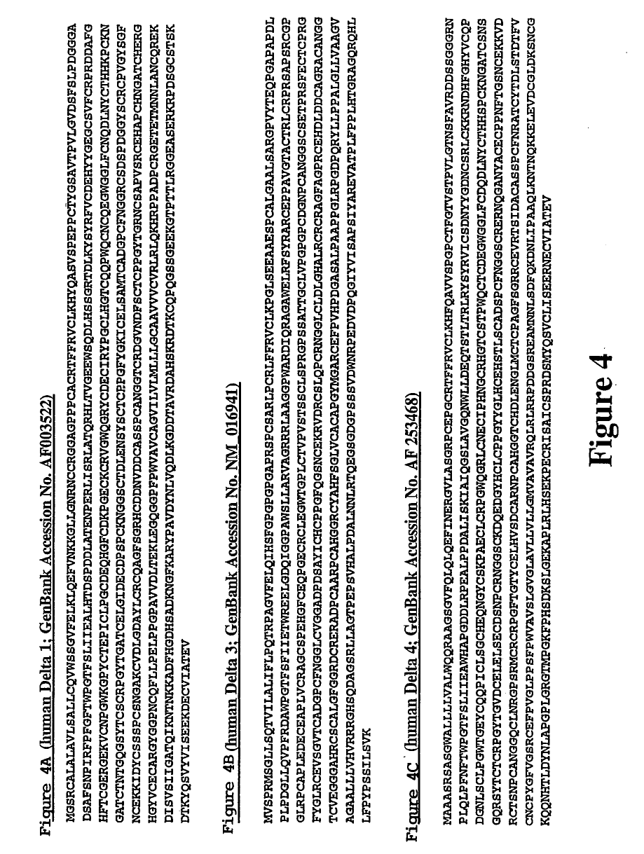

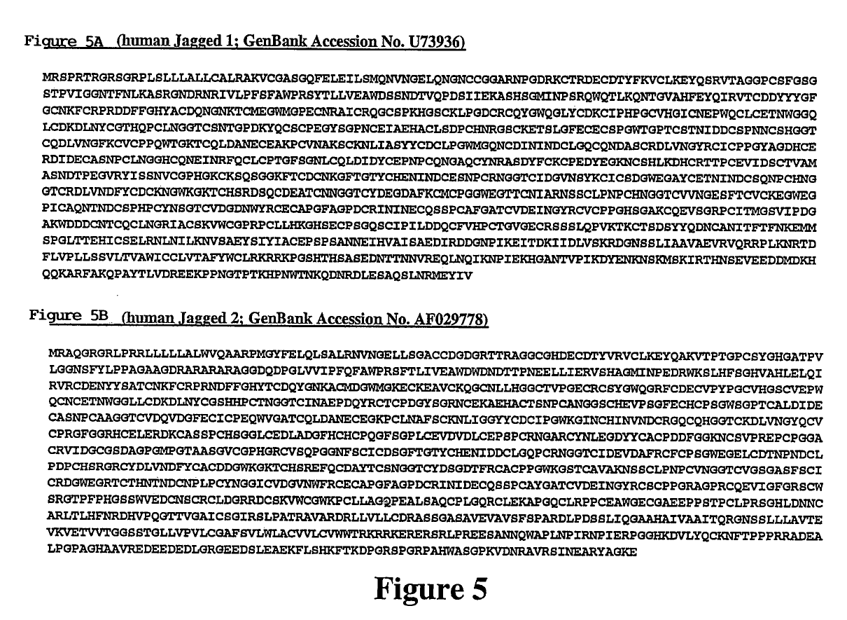

An alignment of DSL domains from Notch ligands from various sources is shown in FIG. 3.

The DSL domain used may be derived from any suitable species, including for example Drosophila, Xenopus, rat, mouse or human. Preferably the DSL domain is derived from a vertebrate, preferably a mammalian, preferably a human Notch ligand sequence.

Thus, for example, a DSL domain for use in the present invention may suitably have at least 30%, preferably at least 50%, preferably at least 60%, preferably at least 70%, preferably at least 80%, preferably at least 90%, preferably at least 95% amino acid sequence identity to the DSL domain of human Jagged 1.

Alternatively a DSL domain for use in the present invention may, for example, have at least 30%, preferably at least 50%, preferably at least 60%, preferably at least 70%, preferably at least 80%, preferably at least 90%, preferably at least 95% amino acid sequence identity to the DSL domain of human Jagged 2.

Alternatively a DSL domain for use in the present invention may, for example, have at least 30%, preferably at least 50%, preferably at least 60%, preferably at least 70%, preferably at least 80%, preferably at least 90%, preferably at least 95% amino acid sequence identity to the DSL domain of human Delta 1.

Alternatively a DSL domain for use in the present invention may, for example, have at least 30%, preferably at least 50%, preferably at least 60%, preferably at least 70%, preferably at least 80%, preferably at least 90%, preferably at least 95% amino acid sequence identity to the DSL domain of human Delta 3.

Alternatively a DSL domain for use in the present invention may, for example, have at least 30%, preferably at least 50%, preferably at least 60%, preferably at least 70%, preferably at least 80%, preferably at least 90%, preferably at least 95% amino acid sequence identity to the DSL domain of human Delta 4.

EGF-Like Domain

The EGF-like motif has been found in a variety of proteins, as well as EGF and Notch and Notch ligands, including those involved in the blood clotting cascade (Furie and Furie, 1988, Cell 53: 505-518). For example, this motif has been found in extracellular proteins such as the blood clotting factors IX and X (Rees et al., 1988, EMBO J. 7:2053-2061; Furie and Furie, 1988, Cell 53: 505-518), in other Drosophila genes (Knust et al., 1987 EMBO J. 761-766; Rothberg et al., 1988, Cell 55:1047-1059), and in some cell-surface receptor proteins, such as thrombomodulin (Suzuki et al., 1987, EMBO J. 6:1891-1897) and LDL receptor (Sudhof et al., 1985, Science 228:815-822). A protein binding site has been mapped to the EGF repeat domain in thrombomodulin and urokinase (Kurosawa et al., 1988, J. Biol. Chem 263:5993-5996; Appella et al., 1987, J. Biol. Chem. 262:4437-4440).

As reported by PROSITE the EGF domain typically includes six cysteine residues which have been shown (in EGF) to be involved in disulfide bonds. The main structure is proposed, but not necessarily required, to be a two-stranded beta-sheet followed by a loop to a C-terminal short two-stranded sheet. Subdomains between the conserved cysteines strongly vary in length as shown in the following schematic representation of a typical EGF-like domain: 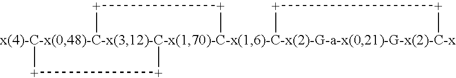
wherein:

‘C’: conserved cysteine involved in a disulfide bond.

‘G’: often conserved glycine

‘a’: often conserved aromatic amino acid

‘x’: any residue

The region between the 5th and 6th cysteine contains two conserved glycines of which at least one is normally present in most EGF-like domains.

The EGF-like domain used may be derived from any suitable species, including for example Drosophila, Xenopus, rat, mouse or human. Preferably the EGF-like domain is derived from a vertebrate, preferably a mammalian, preferably a human Notch ligand sequence.

Suitably, for example, an EGF-like domain for use in the present invention may have at least 30%, preferably at least 50%, preferably at least 60%, preferably at least 70%, preferably at least 80%, preferably at least 90%, preferably at least 95% amino acid sequence identity to an EGF-like domain of human Jagged 1.

Alternatively an EGF-like domain for use in the present invention may, for example, have at least 30%, preferably at least 50%, preferably at least 60%, preferably at least 70%, preferably at least 80%, preferably at least 90%, preferably at least 95% amino acid sequence identity to an EGF-like domain of human Jagged 2.

Alternatively an EGF-like domain for use in the present invention may, for example, have at least 30%, preferably at least 50%, preferably at least 60%, preferably at least 70%, preferably at least 80%, preferably at least 90%, preferably at least 95% amino acid sequence identity to an EGF-like domain of human Delta 1.

Alternatively an EGF-like domain for use in the present invention may, for example, have at least 30%, preferably at least 50%, preferably at least 60%, preferably at least 70%, preferably at least 80%, preferably at least 90%, preferably at least 95% amino acid sequence identity to an EGF-like domain of human Delta 3.

Alternatively an EGF-like domain for use in the present invention may, for example, have at least 30%, preferably at least 50%, preferably at least 60%, preferably at least 70%, preferably at least 80%, preferably at least 90%, preferably at least 95% amino acid sequence identity to an EGF-like domain of human Delta 4.

As a practical matter, whether any particular amino acid sequence is at least X % identical to another sequence can be determined conventionally using known computer programs. For example, the best overall match between a query sequence and a subject sequence, also referred to as a global sequence alignment, can be determined using a program such as the FASTDB computer program based on the algorithm of Brutlag et al. (Comp. App. Biosci. (1990) 6:237-245). In a sequence alignment the query and subject sequences are either both nucleotide sequences or both amino acid sequences. The result of the global sequence alignment is given as percent identity. Alignment scores obtained using the CLUSTALW program may also be used, eg with default settings (see for example Higgins D., Thompson J., Gibson T.Thompson J. D., Higgins D. G., Gibson T. J. (1994). CLUSTAL W: improving the sensitivity of progressivemultiple sequence alignment through sequence weighting, position-specific gap penalties and weight matrix choice. (Nucleic Acids Res. 22:4673-4680).

The term “Notch ligand N-terminal domain” means the part of a Notch ligand sequence from the N-terminus to the start of the DSL domain. It will be appreciated that this term includes sequence variants, fragments, derivatives and mimetics having activity corresponding to naturally occurring domains.

Suitably, for example, a Notch ligand N-terminal domain for use in the present invention may have at least 30%, preferably at least 50%, preferably at least 60%, preferably at least 70%, preferably at least 80%, preferably at least 90%, preferably at least 95% amino acid sequence identity to a Notch ligand N-terminal domain of human Jagged 1.

Alternatively a Notch ligand N-terminal domain for use in the present invention may, for example, have at least 30%, preferably at least 50%, preferably at least 60%, preferably at least 70%, preferably at least 80%, preferably at least 90%, preferably at least 95% amino acid sequence identity to a Notch ligand N-terminal domain of human Jagged 2.

Alternatively a Notch ligand N-terminal domain for use in the present invention may, for example, have at least 30%, preferably at least 50%, preferably at least 60%, preferably at least 70%, preferably at least 80%, preferably at least 90%, preferably at least 95% amino acid sequence identity to a Notch ligand N-terminal domain of human Delta 1.

Alternatively a Notch ligand N-terminal domain for use in the present invention may, for example, have at least 30%, preferably at least 50%, preferably at least 60%, preferably at least 70%, preferably at least 80%, preferably at least 90%, preferably at least 95% amino acid sequence identity to a Notch ligand N-terminal domain of human Delta 3.

Alternatively a Notch ligand N-terminal domain for use in the present invention may, for example, have at least 30%, preferably at least 50%, preferably at least 60%, preferably at least 70%, preferably at least 80%, preferably at least 90%, preferably at least 95% amino acid sequence identity to a Notch ligand N-terminal domain of human Delta 4.

The term “heterologous amino acid sequence” or “heterologous nucleotide sequence” as used herein means a sequence which is not found in the native sequence (eg in the case of a Notch ligand sequence is not found in the native Notch ligand sequence) or its coding sequence. Typically, for example, such a sequence may be an IgFc domain or a tag such as a V5His tag.

By polypeptide for Notch signalling activation is also meant any polypeptide expressed as a result of Notch activation and any polypeptides involved in the expression of such polypeptides, or polynucleotides coding for such polypeptides.

By a protein which is for Notch signalling inhibition or a polynucleotide encoding such a protein, we mean a molecule which is capable of inhibiting Notch, the Notch signalling pathway or any one or more of the components of the Notch signalling pathway.

In one embodiment a modulator of Notch signalling may be a molecule which is capable of modulating Notch-Notch ligand interactions. A molecule may be considered to modulate Notch-Notch ligand interactions if it is capable of inhibiting the interaction of Notch with ligands, preferably to an extent sufficient to provide therapeutic efficacy.

Any one or more of appropriate targets—such as an amino acid sequence and/or nucleotide sequence—may be used for identifying a compound capable of modulating the Notch signalling pathway in any of a variety of drug screening techniques. The target employed in such a test may be free in solution, affixed to a solid support, borne on a cell surface, or located intracellularly.

Techniques for drug screening may be based on the method described in Geysen, European Patent No. 0138855, published on Sep. 13, 1984. In summary, large numbers of different small peptide candidate modulators or targeting molecules are synthesized on a solid substrate, such as plastic pins or some other surface. The peptide test compounds are reacted with a suitable target or fragment thereof and washed. Bound entities are then detected—such as by appropriately adapting methods well known in the art. A purified target can also be coated directly onto plates for use in drug screening techniques. Plates of use for high throughput screening (HTS) will be multi-well plates, preferably having 96, 384 or over 384 wells/plate. Cells can also be spread as “lawns”. Alternatively, non-neutralising antibodies can be used to capture the peptide and immobilise it on a solid support. High throughput screening, as described above for synthetic compounds, can also be used for identifying organic candidate modulators and targeting molecules.

This invention also contemplates the use of competitive drug screening assays in which neutralising antibodies capable of binding a target specifically compete with a test compound for binding to a target.

Techniques are well known in the art for the screening and development of agents such as antibodies, peptidomimetics and small organic molecules which are capable of binding to components of the Notch signalling pathway such as Notch receptors. These include the use of phage display systems for expressing signalling proteins, and using a culture of transfected E. coli or other microorganism to produce the proteins for binding studies of potential binding compounds (see, for example, G. Cesarini, FEBS Letters, 307(l):66-70 (July 1992); H. Gram et al., J. Immunol. Meth., 161:169-176 (1993); and C. Summer et al., Proc. Natl. Acad. Sci., USA, 89:3756-3760 (May 1992)). Further library and screening techniques are described, for example, in U.S. Pat. No. 6,281,344 (Phylos).

Notch Signalling Transduction and Notch Receptor Activation

Notch was first described in Drosophila as a transmembrane protein that functions as a receptor for two different ligands, Delta and Serrate. Vertebrates express multiple Notch receptors and ligands (discussed below). At least four Notch receptors (Notch-1, Notch-2, Notch-3 and Notch-4) have been identified to date in human cells (see for example GenBank Accession Nos. AF308602, AF308601 and U95299—Homo sapiens). For example, sequences of human Notch1 and Notch2 are shown in the Figures hereto.

Notch proteins are synthesized as single polypeptide precursors that undergo cleavage via a Furin-like convertase that yields two polypeptide chains that are further processed to form the mature receptor. The Notch receptor present in the plasma membrane comprises a heterodimer of two Notch proteolytic cleavage products, one comprising an N-terminal fragment consisting of a portion of the extracellular domain, the transmembrane domain and the intracellular domain, and the other comprising the majority of the extracellular domain. The proteolytic cleavage step of Notch to activate the receptor occurs in the Golgi apparatus and is mediated by a furin-like convertase.

Notch receptors are inserted into the membrane as heterodimeric molecules comprising an extracellular domain containing up to 36 epidermal growth factor (EGF)-like repeats [Notch 1/2=36, Notch 3=34 and Notch 4=29], 3 Cysteine Rich Repeats (Lin-Notch (L/N) repeats) and a transmembrane subunit that contains the cytoplasmic domain. The cytoplasmic domain of Notch contains six ankyrin-like repeats, a polyglutamine stretch (OPA) and a PEST sequence. A further domain termed RAM23 lies proximal to the ankyrin repeats and is involved in binding to a transcription factor, known as Suppressor of Hairless [Su(H)] in Drosophila and CBF1 in vertebrates (Tamura K, et al. (1995) Curr. Biol. 5:1416-1423 (Tamura)). The Notch ligands also display multiple EGF-like repeats in their extracellular domains together with a cysteine-rich DSL (Delta-Serrate Lag2) domain that is characteristic of all Notch ligands (Artavanis-Tsakomas et al. (1995) Science 268:225-232, Artavanis-Tsakomas et al. (1999) Science 284:770-776).

The Notch receptor is activated by binding of extracellular ligands, such as Delta and Serrate to the EGF-like repeats of Notch's extracellular domain. Delta may sometimes require cleavage for activation. It may be cleaved by the ADAM disintegrin metalloprotease Kuzbanian at the cell surface, the cleavage event releasing a soluble and active form of Delta. An oncogenic variant of the human Notch-1 protein, also known as TAN-1, which has a truncated extracellular domain, is constitutively active and has been found to be involved in T-cell lymphoblastic leukemias.

The cdc 10/ankyrin intracellular-domain repeats mediate physical interaction with intracellular signal transduction proteins. Most notably, the cdc 10/ankyrin repeats interact with Suppressor of Hairless [Su(H)]. Su(H) is the Drosophila homologue of C-promoter binding factor-1 [CBF-1], a mammalian DNA binding protein involved in the Epstein-Barr virus-induced immortalization of B-cells. It has been demonstrated that, at least in cultured cells, Su(H) associates with the cdc 10/ankyrin repeats in the cytoplasm and translocates into the nucleus upon the interaction of the Notch receptor with its ligand Delta on adjacent cells. Su(H) includes responsive elements found in the promoters of several genes and has been found to be a critical downstream protein in the Notch signalling pathway. The involvement of Su(H) in transcription is thought to be modulated by Hairless.

The intracellular domain of Notch (NotchIC) also has a direct nuclear function (Lieber et al. (1993) Genes Dev 7(10):1949-65 (Lieber)). Recent studies have indeed shown that Notch activation requires that the six cdc 10/ankyrin repeats of the Notch intracellular domain reach the nucleus and participate in transcriptional activation. The site of proteolytic cleavage on the intracellular tail of Notch has been identified between gly1743 and val1744 (termed site 3, or S3) (Schroeter, E. H. et al. (1998) Nature 393(6683):382-6 (Schroeter)). It is thought that the proteolytic cleavage step that releases the cdc 10/ankyrin repeats for nuclear entry is dependent on Presenilin activity.

The intracellular domain has been shown to accumulate in the nucleus where it forms a transcriptional activator complex with the CSL family protein CBF1 (suppressor of hairless, Su(H) in Drosophila, Lag-2 in C. elegans) (Schroeter; Struhl, G. et al. (1998) Cell 93(4):649-60 (Struhl)). The NotchIC-CBF1 complexes then activate target genes, such as the bHLH proteins HES (hairy-enhancer of split like) 1 and 5 (Weinmaster G. (2000) Curr. Opin. Genet. Dev. 10:363-369 (Weinmaster)). This nuclear function of Notch has also been shown for the mammalian Notch homologue (Lu, F. M. et al. (1996) Proc Natl Acad Sci 93(11):5663-7 (Lu)).

S3 processing occurs only in response to binding of Notch ligands Delta or Serrate/Jagged. The post-translational modification of the nascent Notch receptor in the Golgi (Munro S, Freeman M. (2000) Curr. Biol. 10:813-820 (Munro); Ju B J, et al. (2000) Nature 405:191-195 (Ju)) appears, at least in part, to control which of the two types of ligand is expressed on a cell surface. The Notch receptor is modified on its extracellular domain by Fringe, a glycosyl transferase enzyme that binds to the Lin/Notch motif. Fringe modifies Notch by adding O-linked fucose groups to the EGF-like repeats (Moloney D J, et al. (2000) Nature 406:369-375 (Moloney), Brucker K, et al. (2000) Nature 406:411-415 (Brucker)). This modification by Fringe does not prevent ligand binding, but may influence ligand induced conformational changes in Notch. Furthermore, recent studies suggest that the action of Fringe modifies Notch to prevent it from interacting functionally with Serrate/Jagged ligands but allow it to preferentially bind Delta (Panin V M, et al. (1997) Nature 387:908-912 (Panin), Hicks C, et al. (2000) Nat. Cell. Biol. 2:515-520 (Hicks)). Although Drosophila has a single Fringe gene, vertebrates are known to express multiple genes (Radical, Manic and Lunatic Fringes) (Irvine K D (1999) Curr. Opin. Genet. Devel. 9:434-441 (Irvine)).

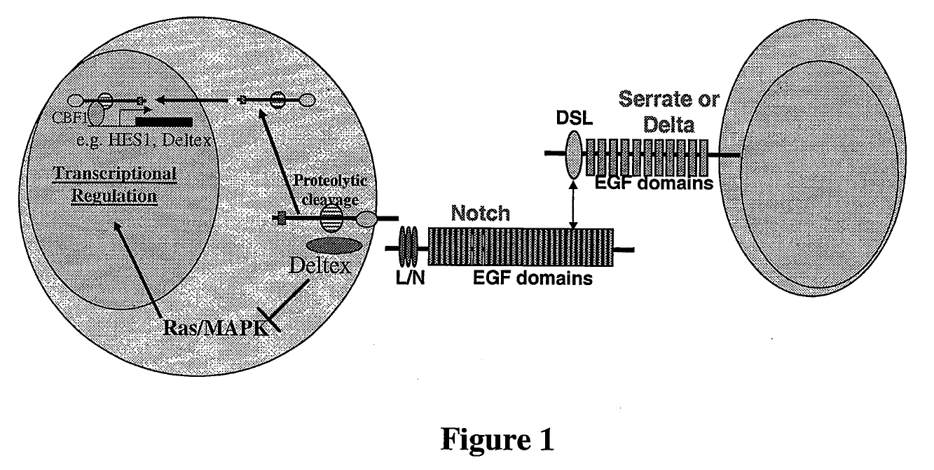

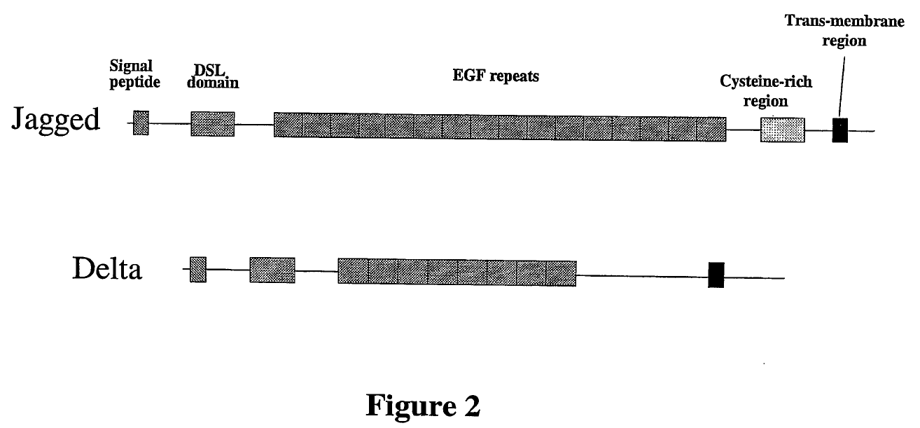

Signal transduction from the Notch receptor can occur via two different pathways (see e.g. FIG. 1). The better defined pathway involves proteolytic cleavage of the intracellular domain of Notch (Notch IC) that translocates to the nucleus and forms a transcriptional activator complex with the CSL family protein CBF1 (suppressor of Hairless, Su(H) in Drosophila, Lag-2 in C. elegans). NotchIC-CBF1 complexes then activate target genes, such as the bHLH proteins HES (hairy-enhancer of split like) 1 and 5. Notch can also signal in a CBF1-independent manner that involves the cytoplasmic zinc finger containing protein Deltex. Unlike CBF1, Deltex does not move to the nucleus following Notch activation but instead can interact with Grb2 and modulate the Ras-JNK signalling pathway.

Target genes of the Notch signalling pathway include Deltex, genes of the Hes family (Hes-1 in particular), Enhancer of Split [E(spl)] complex genes, IL-10, CD-23, CD-4 and D11-1.

Deltex, an intracellular docking protein, replaces Su(H) as it leaves its site of interaction with the intracellular tail of Notch. Deltex is a cytoplasmic protein containing a zinc-finger (Artavanis-Tsakomas et al. (1995) Science 268:225-232; Artavanis-Tsakomas et al. (1999) Science 284:770-776; Osborne B, Miele L. (1999) Immunity 11:653-663 (Osborne)). It interacts with the ankyrin repeats of the Notch intracellular domain. Studies indicate that Deltex promotes Notch pathway activation by interacting with Grb2 and modulating the Ras-JNK signalling pathway (Matsuno et al. (1995) Development 121(8):2633-44; Matsuno K, et al. (1998) Nat. Genet. 19:74-78). Deltex also acts as a docking protein which prevents Su(H) from binding to the intracellular tail of Notch (Matsuno). Thus, Su(H) is released into the nucleus where it acts as a transcriptional modulator. Recent evidence also suggests that, in a vertebrate B-cell system, Deltex, rather than the Su(H) homologue CBF1, is responsible for inhibiting E47 function (Ordentlich et al. (1998) Mol. Cell. Biol. 18:2230-2239 (Ordentlich)). Expression of Deltex is upregulated as a result of Notch activation in a positive feedback loop. The sequence of Homo sapiens Deltex (DTX1) mRNA may be found in GenBank Accession No. AF053700.

Hes-1 (Hairy-enhancer of Split-1) (Takebayashi K. et al. (1994) J Biol Chem 269(7):150-6 (Takebayashi)) is a transcriptional factor with a basic helix-loop-helix structure. It binds to an important functional site in the CD4 silencer leading to repression of CD4 gene expression. Thus, Hes-1 is strongly involved in the determination of T-cell fate. Other genes from the Hes family include Hes-5 (mammalian Enhancer of Split homologue), the expression of which is also upregulated by Notch activation, and Hes-3. Expression of Hes-1 is upregulated as a result of Notch activation. The sequence of Mus musculus Hes-1 can be found in GenBank Accession No. D16464.

The E(spl) gene complex [E(spl)-C] (Leimeister C. et al. (1999) Mech Dev 85(1-2):173-7 (Leimeister)) comprises seven genes of which only E(spl) and Groucho show visible phenotypes when mutant. E(spl) was named after its ability to enhance Split mutations, Split being another name for Notch. Indeed, E(spl)-C genes repress Delta through regulation of achaete-scute complex gene expression. Expression of E(spl) is upregulated as a result of Notch activation.

Interleukin-10 (IL-10) was first characterised in the mouse as a factor produced by Th2 cells which was able to suppress cytokine production by Th1 cells. It was then shown that IL-10 was produced by many other cell types including macrophages, keratinocytes, B cells, Th0 and Th1 cells. It shows extensive homology with the Epstein-Barr bcrf1 gene which is now designated viral IL-10. Although a few immunostimulatory effects have been reported, it is mainly considered as an immunosuppressive cytokine. Inhibition of T cell responses by IL-10 is mainly mediated through a reduction of accessory functions of antigen presenting cells. IL-10 has notably been reported to suppress the production of numerous pro-inflammatory cytokines by macrophages and to inhibit co-stimulatory molecules and MHC class II expression. IL-10 also exerts anti-inflammatory effects on other myeloid cells such as neutrophils and eosinophils. On B cells, IL-10 influences isotype switching and proliferation. More recently, IL-10 was reported to play a role in the induction of regulatory T cells and as a possible mediator of their suppressive effect. Although it is not clear whether it is a direct downstream target of the Notch signalling pathway, its expression has been found to be strongly up-regulated coincident with Notch activation. The mRNA sequence of IL-10 may be found in GenBank ref. No. GI1041812.

CD-23 is the human leukocyte differentiation antigen CD23 (FCE2) which is a key molecule for B-cell activation and growth. It is the low-affinity receptor for IgE. Furthermore, the truncated molecule can be secreted, then functioning as a potent mitogenic growth factor. The sequence for CD-23 may be found in GenBank ref. No. GI1783344.

CTLA4 (cytotoxic T-lymphocyte activated protein 4) is an accessory molecule found on the surface of T-cells which is thought to play a role in the regulation of airway inflammatory cell recruitment and T-helper cell differentiation after allergen inhalation. The promoter region of the gene encoding CTLA4 has CBF1 response elements and its expression is upregulated as a result of Notch activation. The sequence of CTLA4 can be found in GenBank Accession No. L15006.

Dlx-1 (distalless-1) (McGuinness T. Et al (1996) Genomics 35(3):473-85 (McGuiness)) expression is downregulated as a result of Notch activation. Sequences for Dlx genes may be found in GenBank Accession Nos. U51000-3.

CD-4 expression is downregulated as a result of Notch activation. A sequence for the CD-4 antigen may be found in GenBank Accession No. XM006966.

Other genes involved in the Notch signaling pathway, such as Numb, Mastermind and Dsh, and all genes the expression of which is modulated by Notch activation, are included in the scope of this invention.

As described above the Notch receptor family participates in cell-cell signalling events that influence T cell fate decisions. In this signalling NotchIC localises to the nucleus and functions as an activated receptor. Mammalian NotchIC interacts with the transcriptional repressor CBF1. It has been proposed that the NotchIC cdc10/ankyrin repeats are essential for this interaction. Hsieh et al (Hsieh et al. (1996) Molecular & Cell Biology 16(3):952-959) suggests rather that the N-terminal 114 amino acid region of mouse NotchIC contains the CBF1 interactive domain. It is also proposed that NotchIC acts by targeting DNA-bound CBF1 within the nucleus and abolishing CBF1-mediated repression through masking of the repression domain. It is known that Epstein Barr virus (EBV) immortalizing protein EBNA” also utilises CBF1 tethering and masking of repression to upregulate expression of CBF1-repressed B-cell genes. Thus, mimicry of Notch signal transduction is involved in EBV-driven immortalization. Strobl et al (Strobl et al. (2000) J Virol 74(4):1727-35) similarly reports that “EBNA2 may hence be regarded as a functional equivalent of an activated Notch receptor”. Other EBV proteins which fall in this category include BARF0 (Kusano and Raab-Truab (2001) J Virol 75(1):384-395 (Kusano and Raab-Traub)) and LMP2A.

Notch Ligands and Homologues

As noted above, examples of mammalian Notch ligands identified to date include the Delta family, for example Delta-1 (Genbank Accession No. AF003522—Homo sapiens), Delta-3 (Genbank Accession No. AF084576—Rattus norvegicus) and Delta-like 3 (Mus musculus), the Serrate family, for example Serrate-1 and Serrate-2 (WO97/01571, WO96/27610 and WO92/19734), Jagged-1 and Jagged-2 (Genbank Accession No. AF029778—Homo sapiens), and LAG-2. Homology between family members is extensive.

By a “homologue” is meant a gene product that exhibits sequence homology, either amino acid or nucleic acid sequence homology, to any one of the known Notch ligands, for example as mentioned above. Typically, a homologue of a known Notch ligand will be at least 20%, preferably at least 30%, identical at the amino acid level to the corresponding known Notch ligand over a sequnce of at least 10, preferably at least 20, preferably at least 50, suitably at least 100 amino acids, or over the entire length of the Notch ligand. Techniques and software for calculating sequence homology between two or more amino acid or nucleic acid sequences are well known in the art (see for example http://www.ncbi.nlm.nih.gov and Ausubel et al., Current Protocols in Molecular Biology (1995), John Wiley & Sons, Inc.)

As noted above, Notch ligands identified to date have a diagnostic DSL domain (D. Delta, S. Serrate, L. Lag2) comprising 20 to 22 amino acids at the amino terminus of the protein and up to 14 or more EGF-like repeats on the extracellular surface. It is therefore preferred that homologues of Notch ligands also comprise a DSL domain at the N-terminus and up to 14 or more EGF-like repeats on the extracellular surface.

In addition, suitable homologues will preferably be capable of binding to a Notch receptor. Binding may be assessed by a variety of techniques known in the art including in vitro binding assays and activation of the receptor (in the case of an agonist or partial agonist) may be determined for example by use of reporter assays as described in the Examples hereto and in WO 03/012441 (Lorantis) the text of which is hereby incorporated herein by reference.

Homologues of Notch ligands can be identified in a number of ways, for example by probing genomic or cDNA libraries with probes comprising all or part of a nucleic acid encoding a Notch ligand under conditions of medium to high stringency (for example 0.03M sodium chloride and 0.03M sodium citrate at from about 50° C. to about 60° C.). Alternatively, homologues may also be obtained using degenerate PCR which will generally use primers designed to target sequences within the variants and homologues encoding conserved amino acid sequences. The primers will contain one or more degenerate positions and will be used at stringency conditions lower than those used for cloning sequences with single sequence primers against known sequences.

Polypeptide substances may be purified from mammalian cells, obtained by recombinant expression in suitable host cells or obtained commercially. Alternatively, nucleic acid constructs encoding the polypeptides may be used. As a further example, overexpression of Notch or Notch ligand, such as Delta or Serrate, may be brought about by introduction of a nucleic acid construct capable of activating the endogenous gene, such as the Serrate or Delta gene. In particular, gene activation can be achieved by the use of homologous recombination to insert a heterologous promoter in place of the natural promoter, such as the Serrate or Delta promoter, in the genome of the target cell.

The activating molecule of the present invention may, in an alternative embodiment, be capable of modifying Notch-protein expression or presentation on the cell membrane or signalling pathways. Agents that enhance the presentation of a fully functional Notch-protein on the target cell surface include matrix metalloproteinases such as the product of the Kuzbanian gene of Drosophila (Dkuz et al. (1997) Cell 90: 271-280 (Dkuz)) and other ADAMALYSIN gene family members.

Polypeptides, Proteins and Amino Acid Sequences

As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “protein”.

“Peptide” usually refers to a short amino acid sequence that is 10 to 40 amino acids long, preferably 10 to 35 amino acids.

The amino acid sequence may be prepared and isolated from a suitable source, or it may be made synthetically or it may be prepared by use of recombinant DNA techniques.

Within the definitions of “proteins” useful in the present invention, the specific amino acid residues may be modified in such a manner that the protein in question retains at least one of its endogenous functions, such modified proteins are referred to as “variants”. A variant protein can be modified by addition, deletion and/or substitution of at least one amino acid present in the naturally-occurring protein.

Typically, amino acid substitutions may be made, for example from 1, 2 or 3 to 10 or 20 substitutions provided that the modified sequence retains the required target activity or ability to modulate Notch signalling. Amino acid substitutions may include the use of non-naturally occurring analogues.

Proteins of use in the present invention may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity, and/or the amphipathic nature of the residues as long as the target or modulation function is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

For ease of reference, the one and three letter codes for the main naturally occurring amino acids (and their associated codons) are set out below:

| | | | |
|---|---|---|---|
| Symbol | 3-letter | Meaning | Codons |

| | | | |
|---|---|---|---|
| A | Ala | Alanine | GCT, GCC, GCA, GCG |
| B | Asp, Asn | Aspartic | GAT, GAC, AAT, AAC |
| | | Asparagine | |
| C | Cys | Cysteine | TGT, TGC |
| D | Asp | Aspartic | GAT, GAC |
| E | Glu | Glutamic | GAA, GAG |
| F | Phe | Phenylalanine | TTT, TTC |
| G | Gly | Glycine | GGT, GGC, GGA, GGG |
| H | His | Histidine | CAT, CAC |
| I | Ile | Isoleucine | ATT, ATC, ATA |
| K | Lys | Lysine | AAA, AAG |
| L | Leu | Leucine | TTG, TTA, CTT, CTC, |
| | | CTA, CTG | |
| M | Met | Methionine | ATG |
| N | Asn | Asparagine | AAT, AAC |
| P | Pro | Proline | CCT, CCC, CCA, CCG |
| Q | Gln | Glutamine | CAA, CAG |
| R | Arg | Arginine | CGT, CGC, CGA, CGG, |
| | | AGA, AGG | |
| S | Ser | Serine | TCT, TCC, TCA, TCG, |
| | | AGT, AGC | |
| T | Thr | Threonine | ACT, ACC, ACA, ACG |
| V | Val | Valine | GTT, GTC, GTA, GTG |
| W | Trp | Tryptophan | TGG |
| X | Xxx | Unknown | |
| Y | Tyr | Tyrosine | TAT, TAC |
| Z | Glu, Gln | Glutamic, | GAA, GAG, CAA, CAG |
| | | Glutamine | |
| * | End | Terminator | TAA, TAG, TGA |

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

As used herein, the term “protein” includes single-chain polypeptide molecules as well as multiple-polypeptide complexes where individual constituent polypeptides are linked by covalent or non-covalent means. As used herein, the terms “polypeptide” and “peptide” refer to a polymer in which the monomers are amino acids and are joined together through peptide or disulfide bonds. The terms subunit and domain may also refer to polypeptides and peptides having biological function. A peptide useful in the invention will at least have a target or signalling modulation capability. “Fragments” are also variants and the term typically refers to a selected region of the protein that is of interest in a binding assay and for which a binding partner is known or determinable. “Fragment” thus refers to an amino acid sequence that is a portion of a full-length polypeptide, for example between about 8 and about 1500 amino acids in length, typically between about 8 and about 745 amino acids in length, preferably about 8 to about 300, more preferably about 8 to about 200 amino acids, and even more preferably about 10 to about 50 or 100 amino acids in length. “Peptide” preferably refers to a short amino acid sequence that is 10 to 40 amino acids long, preferably 10 to 35 amino acids.

Such variants may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded protein. These methods are only illustrative of the numerous standard techniques known in the art for manipulation of DNA sequences and other known techniques may also be used.

Variants of the nucleotide sequence may also be made. Such variants will preferably comprise codon optimised sequences. Codon optimisation is known in the art as a method of enhancing RNA stability and therefore gene expression. The redundancy of the genetic code means that several different codons may encode the same amino acid. For example, leucine, arginine and serine are each encoded by six different codons. Different organisms show preferences in their use of the different codons. Viruses such as HIV, for instance, use a large number of rare codons. By changing a nucleotide sequence such that rare codons are replaced by the corresponding commonly used mammalian codons, increased expression of the sequences in mammalian target cells can be achieved. Codon usage tables are known in the art for mammalian cells, as well as for a variety of other organisms.

Proteins or polypeptides may be in the form of the “mature” protein or may be a part of a larger protein such as a fusion protein or precursor. For example, it is often advantageous to include an additional amino acid sequence which contains secretory or leader sequences or pro-sequences (such as a HIS oligomer, immunoglobulin Fc, glutathione S-transferase, FLAG etc) to aid in purification. Likewise such an additional sequence may sometimes be desirable to provide added stability during recombinant production. In such cases the additional sequence may be cleaved (eg chemically or enzymatically) to yield the final product. In some cases, however, the additional sequence may also confer a desirable pharmacological profile (as in the case of IgFc fusion proteins) in which case it may be preferred that the additional sequence is not removed so that it is present in the final product as administered.

Where the modulator of Notch signalling or antigen/antigenic determinant comprises a nucleotide sequence it may suitably be codon optimised for expression in mammalian cells. In a preferred embodiment, such sequences are optimised in their entirety.

Nucleic Acids and Polynucleotides

“Polynucleotide” refers to a polymeric form of nucleotides of at least 10 bases in length and up to 10,000 bases or more, either ribonucleotides or deoxyribonucleotides or a modified form of either type of nucleotide. The term includes single and double stranded forms of DNA and RNA and also derivatised versions such as protein nucleic acid (PNA).

These may be constructed using standard recombinant DNA methodologies. The nucleic acid may be RNA or DNA and is preferably DNA. Where it is RNA, manipulations may be performed via cDNA intermediates. Generally, a nucleic acid sequence encoding the first region will be prepared and suitable restriction sites provided at the 5′ and/or 3′ ends. Conveniently the sequence is manipulated in a standard laboratory vector, such as a plasmid vector based on pBR322 or pUC19 (see below). Reference may be made to Molecular Cloning by Sambrook et al. (Cold Spring Harbor, 1989) or similar standard reference books for exact details of the appropriate techniques.

Nucleic acid encoding the second region may likewise be provided in a similar vector system.

Sources of nucleic acid may be ascertained by reference to published literature or databanks such as GenBank. Nucleic acid encoding the desired first or second sequences may be obtained from academic or commercial sources where such sources are willing to provide the material or by synthesising or cloning the appropriate sequence where only the sequence data are available. Generally this may be done by reference to literature sources which describe the cloning of the gene in question.

Alternatively, where limited sequence data are available or where it is desired to express a nucleic acid homologous or otherwise related to a known nucleic acid, exemplary nucleic acids can be characterised as those nucleotide sequences which hybridise to the nucleic acid sequences known in the art.

It will be understood by a skilled person that numerous different nucleotide sequences can encode the same protein used in the present invention as a result of the degeneracy of the genetic code. In addition, it is to be understood that skilled persons may, using routine techniques, make nucleotide substitutions that do not affect the protein encoded by the nucleotide sequence of the present invention to reflect the codon usage of any particular host organism in which the target protein or protein for Notch signalling modulation of the present invention is to be expressed.

In general, the terms “variant”, “homologue” or “derivative” in relation to the nucleotide sequence used in the present invention includes any substitution of, variation of, modification of, replacement of, deletion of or addition of one (or more) nucleic acid from or to the sequence providing the resultant nucleotide sequence codes for a modulator of Notch signalling.

As indicated above, with respect to sequence homology, preferably there is at least 40%, preferably at least 70%, preferably at least 75%, more preferably at least 85%, more preferably at least 90% homology to the reference sequences. More preferably there is at least 95%, more preferably at least 98%, homology. Nucleotide homology comparisons may be conducted as described above. A preferred sequence comparison program is the GCG Wisconsin Bestfit program described above. The default scoring matrix has a match value of 10 for each identical nucleotide and −9 for each mismatch. The default gap creation penalty is −50 and the default gap extension penalty is −3 for each nucleotide.

The present invention also encompasses nucleotide sequences that are capable of hybridising selectively to the reference sequences, or any variant, fragment or derivative thereof, or to the complement of any of the above. Nucleotide sequences are preferably at least 15 nucleotides in length, more preferably at least 20, 30, 40 or 50 nucleotides in length.

The term “hybridization” as used herein shall include “the process by which a strand of nucleic acid joins with a complementary strand through base pairing” as well as the process of amplification as carried out in polymerase chain reaction (PCR) technologies.

Nucleotide sequences useful in the invention capable of selectively hybridising to the nucleotide sequences presented herein, or to their complement, will be generally at least 75%, preferably at least 85 or 90% and more preferably at least 95% or 98% homologous to the corresponding nucleotide sequences presented herein over a region of at least 20, preferably at least 25 or 30, for instance at least 40, 60 or 100 or more contiguous nucleotides. Preferred nucleotide sequences of the invention will comprise regions homologous to the nucleotide sequence, preferably at least 80 or 90% and more preferably at least 95% homologous to the nucleotide sequence.

The term “selectively hybridizable” means that the nucleotide sequence used as a probe is used under conditions where a target nucleotide sequence of the invention is found to hybridize to the probe at a level significantly above background. The background hybridization may occur because of other nucleotide sequences present, for example, in the cDNA or genomic DNA library being screened. In this event, background implies a level of signal generated by interaction between the probe and a non-specific DNA member of the library which is less than 10 fold, preferably less than 100 fold as intense as the specific interaction observed with the target DNA. The intensity of interaction may be measured, for example, by radiolabelling the probe, e.g. with ³²P.

Hybridization conditions are based on the melting temperature (Tm) of the nucleic acid binding complex, as taught in Berger and Kimmel (1987, Guide to Molecular Cloning Techniques, Methods in Enzymology, Vol 152, Academic Press, San Diego Calif.), and confer a defined “stringency” as explained below.

Maximum stringency typically occurs at about Tm-5° C. (5° C. below the Tm of the probe); high stringency at about 5° C. to 10° C. below Tm; intermediate stringency at about 10° C. to 20° C. below Tm; and low stringency at about 20° C. to 25° C. below Tm. As will be understood by those of skill in the art, a maximum stringency hybridization can be used to identify or detect identical nucleotide sequences while an intermediate (or low) stringency hybridization can be used to identify or detect similar or related polynucleotide sequences.

In a preferred aspect, the present invention covers nucleotide sequences that can hybridise to the nucleotide sequence of the present invention under stringent conditions (e.g. 65° C. and 0.1×SSC {1×SSC=0.15 M NaCl, 0.015 M Na₃ Citrate pH 7.0). Where the nucleotide sequence of the invention is double-stranded, both strands of the duplex, either individually or in combination, are encompassed by the present invention. Where the nucleotide sequence is single-stranded, it is to be understood that the complementary sequence of that nucleotide sequence is also included within the scope of the present invention.

Nucleotide sequences can be obtained in a number of ways. Variants of the sequences described herein may be obtained for example by probing DNA libraries made from a range of sources. In addition, other viral/bacterial, or cellular homologues particularly cellular homologues found in mammalian cells (e.g. rat, mouse, bovine and primate cells), may be obtained and such homologues and fragments thereof in general will be capable of selectively hybridising to the sequences shown in the sequence listing herein. Such sequences may be obtained by probing cDNA libraries made from or genomic DNA libraries from other animal species, and probing such libraries with probes comprising all or part of the reference nucleotide sequence under conditions of medium to high stringency. Similar considerations apply to obtaining species homologues and allelic variants of the amino acid and/or nucleotide sequences useful in the present invention.

Variants and strain/species homologues may also be obtained using degenerate PCR which will use primers designed to target sequences within the variants and homologues encoding conserved amino acid sequences within the sequences of the present invention. Conserved sequences can be predicted, for example, by aligning the amino acid sequences from several variants/homologues. Sequence alignments can be performed using computer software known in the art. For example the GCG Wisconsin PileUp program is widely used. The primers used in degenerate PCR will contain one or more degenerate positions and will be used at stringency conditions lower than those used for cloning sequences with single sequence primers against known sequences.

Alternatively, such nucleotide sequences may be obtained by site directed mutagenesis of characterised sequences. This may be useful where for example silent codon changes are required to sequences to optimise codon preferences for a particular host cell in which the nucleotide sequences are being expressed. Other sequence changes may be desired in order to introduce restriction enzyme recognition sites, or to alter the activity of the modulator of Notch signalling encoded by the nucleotide sequences.

The nucleotide sequences such as a DNA polynucleotides useful in the invention may be produced recombinantly, synthetically, or by any means available to those of skill in the art. They may also be cloned by standard techniques.

In general, primers will be produced by synthetic means, involving a step-wise manufacture of the desired nucleic acid sequence one nucleotide at a time. Techniques for accomplishing this using automated techniques are readily available in the art.

Longer nucleotide sequences will generally be produced using recombinant means, for example using a PCR (polymerase chain reaction) cloning techniques. This will involve making a pair of primers (e.g. of about 15 to 30 nucleotides) flanking a region of the targeting sequence which it is desired to clone, bringing the primers into contact with mRNA or cDNA obtained from an animal or human cell, performing a polymerase chain reaction (PCR) under conditions which bring about amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector. For larger genes, portions may be cloned separately in this way and then ligated to form the complete sequence.

Protein and Polypeptide Expression

For recombinant production, host cells can be genetically engineered to incorporate expression systems or polynucleotides of the invention. Introduction of a polynucleotide into the host cell can be effected by methods described in many standard laboratory manuals, such as Davis et al and Sambrook et al, such as calcium phosphate transfection, DEAE-dextran mediated transfection, transvection, microinjection, cationic lipid-mediated transfection, electroporation, transduction, scrape loading, ballistic introduction and infection. In will be appreciated that such methods can also be employed in vitro or in vivo as drug delivery systems.

Representative examples of appropriate hosts include bacterial cells, such as streptococci, staphylococci, E. coli, streptomyces and Bacillus subtilis cells; fungal cells, such as yeast cells and Aspergillus cells; insect cells such as Drosophila S2 and Spodoptera Sf9 cells; animal cells such as CHO, COS, NSO, HeLa, C127, 3T3, BHK, 293 and Bowes melanoma cells; T-cell lines such as Jurkat cells; B-cell lines such as A20 cells; and plant cells. A great variety of expression systems can be used to produce a polypeptide useful in the present invention. Such vectors include, among others, chromosomal, episomal and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. The expression system constructs may contain control regions that regulate as well as engender expression. Generally, any system or vector suitable to maintain, propagate or express polynucleotides and/or to express a polypeptide in a host may be used for expression in this regard. The appropriate DNA sequence may be inserted into the expression system by any of a variety of well-known and routine techniques, such as, for example, those set forth in Sambrook et al.

For secretion of the translated protein into the lumen of the endoplasmic reticulum, into the periplasmic space or into the extracellular environment, appropriate secretion signals may be incorporated into the expressed polypeptide. These signals may be endogenous to the polypeptide or they may be heterologous signals.

Active agents for use in the invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography is employed for purification. Techniques for refolding protein may be employed to regenerate active conformation when the polypeptide is denatured during isolation and/or purification.

Agents for Notch Signalling Inhibition

Suitably an inhibitor of the Notch signalling pathway may be an agent which interacts with, and preferably binds to a Notch receptor or a Notch ligand so as to interfere with endogenous Notch ligand-receptor interaction (also termed “Notch-Notch ligand interaction”) but does not activate the receptor, or does so to a lesser degree than endogenous Notch ligands. Such an agent may be referred to as a “Notch antagonist” or “Notch receptor antagonist”. Preferably the inhibitor inhibits Notch ligand-receptor interaction in immune cells such as lymphocytes and APCs, preferably in lymphocytes, preferably in T-cells.

Suitably, for example, in one embodiment an inhibitor of Notch signalling for incorporation into a conjugate of the present invention may comprise a protein or polypeptide which comprises a Notch ligand DSL domain and 1 or more Notch ligand EGF-like domains.

Suitably, for example, such an inhibitor of Notch signalling may comprise:

i) a protein or polypeptide which comprises a Notch ligand DSL domain having at least 30%, preferably at least 50% amino acid sequence similarity or identity to the DSL domain of human Delta1, Delta3 or Delta4 and at least one Notch ligand EGF-like domain having at least 30%, preferably at least 50% amino acid sequence similarity or identity to an EGF-like domain of human Delta1, Delta3 or Delta4.

Suitably, for example, an inhibitor of Notch signalling may comprise:

i) a protein or polypeptide which comprises a Notch ligand DSL domain having at least 30%, preferably at least 50% amino acid sequence similarity or identity to the DSL domain of human Delta1, Delta3 or Delta4 and either 0, 1 or 2, but no more than 2 Notch ligand EGF-like domains having at least 30%, preferably at least 50% amino acid sequence similarity or identity to an EGF-like domain of human Delta1, Delta3 or Delta4.

Alternatively, for example, an inhibitor of Notch signalling for use in a conjugate according to the present invention may comprise all or part of a Notch extracellular domain involved in ligand binding, for example a protein or polypeptide which comprises a Notch EGF-like domain, preferably having at least 30%, preferably at least 50% amino acid sequence similarity or identity to an EGF domain of human Notch1, Notch2, Notch3 or Notch4. Preferably at least 2 or more such EGF domains are present. An agent such as this may bind to endogenous Notch ligands and thereby inhibit Notch activation by such ligands.

For example, such an inhibitor of Notch signalling may comprise a protein or polypeptide which comprises a Notch EGF-like domain having at least 30%, preferably at least 50% amino acid sequence similarity or identity to EGF11 of human Notch1, Notch2, Notch3 or Notch4 and a Notch EGF-like domain having at least 30%, preferably at least 50% amino acid sequence similarity or identity to EGF12 of human Notch1, Notch2, Notch3 or Notch4.

For example, a variety of fusion proteins/chimeras comprising extracellular domains of Notch proteins fused to IgFc domains are available for example from R &D Systems, for example as follows: Notch-1 Rat Recombinant Rat Notch-1/Fc Chimera, (Cat No 1057-TK-050); Notch-2 Recombinant Rat Notch-2/Fc Chimera, (Cat No. 1190-NT-050); and Notch-3 Mouse Recombinant Mouse Notch-3/Fc Chimera, (Cat No 1308-NT-050).

Other Notch signalling pathway antagonists/ inhibitors include antibodies which inhibit interactions between components of the Notch signalling pathway, e.g. antibodies to Notch receptors (Notch proteins) or Notch ligands.

Thus, for example, the inhibitor of Notch signaling may be an antibody which binds to a Notch receptor, suitably an antibody which binds to human Notch1, Notch2, Notch3 and/or Notch4, without activating the Notch receptor, and which thereby reduces or prevents activation of the bound receptor by endogenous Notch ligands by interfering with normal Notch-ligand interaction.

Alternatively, for example, the inhibitor of Notch signaling may be an antibody which binds to a Notch ligand, suitably an antibody which binds to human Delta1, Delta3 and/or Delta4 or human Jagged1 and/or Jagged2 and which thereby reduces or prevents interaction of the bound ligand with endogenous Notch receptors by interfering with normal Notch-ligand interaction.

For example, antibodies against Notch and Notch ligands are described in U.S. Pat. No. 5,648,464, U.S. Pat. No. 5,849,869 and U.S. Pat. No. 6,004,924 (Yale University/Imperial Cancer Technology), the texts of which are herein incorporated by reference.

Antibodies generated against the Notch receptor are also described in WO 0020576 (the text of which is also incorporated herein by reference). For example, this document discloses generation of antibodies against the human Notch-1 EGF-like repeats 11 and 12. For example, in particular embodiments, WO 0020576 discloses a monoclonal antibody secreted by a hybridoma designated A6 having the ATCC Accession No. HB12654, a monoclonal antibody secreted by a hybridoma designated C11 having the ATCC Accession No. HB12656 and a monoclonal antibody secreted by a hybridoma designated F3 having the ATCC Accession No. HB 12655.

A

n anti-human-Jagged1 antibody is available from R & D Systems, Inc, reference MAB12771 (Clone 188323).

Other substances which may be used to reduce interaction between Notch and Notch ligands are exogenous Notch or Notch ligands or functional derivatives thereof. Such Notch ligand derivatives would preferably have the DSL domain at the N-terminus and preferably up to about 16 or more, for example between about 1 to 8, preferably 3 to 8 EGF-like repeats on the extracellular surface. A peptide corresponding to the Delta/Serrate/LAG-2 domain of hJagged1 and supernatants from COS cells expressing a soluble form of the extracellular portion of hjagged1 was found to mimic the effect of Jagged1 in inhibiting Notch1 (Li).

Assays

Whether a substance can be used for modulating Notch-Notch ligand expression may be determined using suitable screening assays.

For example, a suitable HES-1/luciferase reporter assay for Notch signaling is described, for example, in Varnum-Finney et al., Journal of Cell Science 113, 4313-4318 (2000) and in Example 6 herein.

Notch signalling can also be monitored either through protein assays or through nucleic acid assays. Activation of the Notch receptor leads to the proteolytic cleavage of its cytoplasmic domain and the translocation thereof into the cell nucleus. The “detectable signal” referred to herein may be any detectable manifestation attributable to the presence of the cleaved intracellular domain of Notch. Thus, increased Notch signalling can be assessed at the protein level by measuring intracellular concentrations of the cleaved Notch domain. Activation of the Notch receptor also catalyses a series of downstream reactions leading to changes in the levels of expression of certain well-defined genes. Thus, increased Notch signalling can be assessed at the nucleic acid level by say measuring intracellular concentrations of specific mRNAs. In one preferred embodiment of the present invention, the assay is a protein assay. In another preferred embodiment of the present invention, the assay is a nucleic acid assay.

The advantage of using a nucleic acid assay is that they are sensitive and that small samples can be analysed.

The intracellular concentration of a particular mRNA, measured at any given time, reflects the level of expression of the corresponding gene at that time. Thus, levels of mRNA of downstream target genes of the Notch signalling pathway can be measured in an indirect assay of the T-cells of the immune system. In particular, an increase in levels of Deltex, Hes-1 and/or IL-10 mRNA may, for instance, indicate induced anergy while an increase in levels of Dll-1 or IFN-γ mRNA, or in the levels of mRNA encoding cytokines such as IL-2, IL-5 and IL-13, may indicate improved responsiveness.

Various nucleic acid assays are known. Any convention technique which is known or which is subsequently disclosed may be employed. Examples of suitable nucleic acid assay are mentioned below and include amplification, PCR, RT-PCR, RNase protection, blotting, spectrometry, reporter gene assays, gene chip arrays and other hybridization methods.

In particular, gene presence, amplification and/or expression may be measured in a sample directly, for example, by conventional Southern blotting, Northern blotting to quantitate the transcription of mRNA, dot blotting (DNA or RNA analysis), or in situ hybridisation, using an appropriately labelled probe. Those skilled in the art will readily envisage how these methods may be modified, if desired.

PCR was originally developed as a means of amplifying DNA from an impure sample. The technique is based on a temperature cycle which repeatedly heats and cools the reaction solution allowing primers to anneal to target sequences and extension of those primers for the formation of duplicate daughter strands. RT-PCR uses an RNA template for generation of a first strand cDNA with a reverse transcriptase. The cDNA is then amplified according to standard PCR protocol. Repeated cycles of synthesis and denaturation result in an exponential increase in the number of copies of the target DNA produced. However, as reaction components become limiting, the rate of amplification decreases until a plateau is reached and there is little or no net increase in PCR product. The higher the starting copy number of the nucleic acid target, the sooner this “end-point” is reached.

Real-time PCR uses probes labeled with a fluorescent tag or fluorescent dyes and differs from end-point PCR for quantitative assays in that it is used to detect PCR products as they accumulate rather than for the measurement of product accumulation after a fixed number of cycles. The reactions are characterized by the point in time during cycling when amplification of a target sequence is first detected through a significant increase in fluorescence.

The ribonuclease protection (RNase protection) assay is an extremely sensitive technique for the quantitation of specific RNAs in solution. The ribonuclease protection assay can be performed on total cellular RNA or poly(A)-selected mRNA as a target. The sensitivity of the ribonuclease protection assay derives from the use of a complementary in vitro transcript probe which is radiolabeled to high specific activity. The probe and target RNA are hybridized in solution, after which the mixture is diluted and treated with ribonuclease (RNase) to degrade all remaining single-stranded RNA. The hybridized portion of the probe will be protected from digestion and can be visualized via electrophoresis of the mixture on a denaturing polyacrylamide gel followed by autoradiography. Since the protected fragments are analyzed by high resolution polyacrylamide gel electrophoresis, the ribonuclease protection assay can be employed to accurately map mRNA features. If the probe is hybridized at a molar excess with respect to the target RNA, then the resulting signal will be directly proportional to the amount of complementary RNA in the sample.

Gene expression may also be detected using a reporter system. Such a reporter system may comprise a readily identifiable marker under the control of an expression system, e.g. of the gene being monitored. Fluorescent markers, which can be detected and sorted by FACS, are preferred. Especially preferred are GFP and luciferase. Another type of preferred reporter is cell surface markers, i.e. proteins expressed on the cell surface and therefore easily identifiable.

In general, reporter constructs useful for detecting Notch signalling by expression of a reporter gene may be constructed according to the general teaching of Sambrook et al. (1989). Typically, constructs according to the invention comprise a promoter by the gene of interest, and a coding sequence encoding the desired reporter constructs, for example of GFP or luciferase. Vectors encoding GFP and luciferase are known in the art and available commercially.

Sorting of cells, based upon detection of expression of genes, may be performed by any technique known in the art, as exemplified above. For example, cells may be sorted by flow cytometry or FACS. For a general reference, see Flow Cytometry and Cell Sorting: A Laboratory Manual (1992) A. Radbruch (Ed.), Springer Laboratory, New York.

Flow cytometry is a powerful method for studying and purifying cells. It has found wide application, particularly in immunology and cell biology: however, the capabilities of the FACS can be applied in many other fields of biology. The acronym F.A.C.S. stands for Fluorescence Activated Cell Sorting, and is used interchangeably with “flow cytometry”. The principle of FACS is that individual cells, held in a thin stream of fluid, are passed through one or more laser beams, causing light to be scattered and fluorescent dyes to emit light at various frequencies. Photomultiplier tubes (PMT) convert light to electrical signals, which are interpreted by software to generate data about the cells. Sub-populations of cells with defined characteristics can be identified and automatically sorted from the suspension at very high purity (˜100%).

FACS can be used to measure gene expression in cells transfected with recombinant DNA encoding polypeptides. This can be achieved directly, by labelling of the protein product, or indirectly by using a reporter gene in the construct. Examples of reporter genes are β-galactosidase and Green Fluorescent Protein (GFP). β-galactosidase activity can be detected by FACS using fluorogenic substrates such as fluorescein digalactoside (FDG). FDG is introduced into cells by hypotonic shock, and is cleaved by the enzyme to generate a fluorescent product, which is trapped within the cell. One enzyme can therefore generate a large amount of fluorescent product. Cells expressing GFP constructs will fluoresce without the addition of a substrate. Mutants of GFP are available which have different excitation frequencies, but which emit fluorescence in the same channel. In a two-laser FACS machine, it is possible to distinguish cells which are excited by the different lasers and therefore assay two transfections at the same time.

Alternative means of cell sorting may also be employed. For example, the invention comprises the use of nucleic acid probes complementary to mRNA. Such probes can be used to identify cells expressing polypeptides individually, such that they may subsequently be sorted either manually, or using FACS sorting. Nucleic acid probes complementary to mRNA may be prepared according to the teaching set forth above, using the general procedures as described by Sambrook et al (1989).

In a preferred embodiment, the invention comprises the use of an antisense nucleic acid molecule, complementary to a mRNA, conjugated to a fluorophore which may be used in FACS cell sorting.

Methods have also been described for obtaining information about gene expression and identity using so-called gene chip arrays or high-density DNA arrays (Chee). These high-density arrays are particularly useful for diagnostic and prognostic purposes. Use may also be made of In vivo Expression Technology (IVET) (Camilli). IVET identifies genes up-regulated during say treatment or disease when compared to laboratory culture.

The advantage of using a protein assay is that Notch activation can be directly measured. Assay techniques that can be used to determine levels of a polypeptide are well known to those skilled in the art. Such assay methods include radioimmunoassays, competitive-binding assays, Western Blot analysis, antibody sandwich assays, antibody detection, FACS and ELISA assays.

As described above the modulator of Notch signalling may also be an immune cell which has been treated to modulate expression or interaction of Notch, a Notch ligand or the Notch signalling pathway. Such cells may readily be prepared, for example, as described in WO 00/36089 in the name of Lorantis Ltd, the text of which is herein incorporated by reference.

Preparation of Primed APCs and Lymphocytes

According to one aspect of the invention immune cells may be used to present antigens or allergens and/or may be treated to modulate expression or interaction of Notch, a Notch ligand or the Notch signalling pathway. Thus, for example, Antigen Presenting Cells (APCs) may be cultured in a suitable culture medium such as DMEM or other defined media, optionally in the presence of fetal calf serum. Cytokines, if present, are typically added at up to 1000 U/ml. Optimum concentrations may be determined by titration. One or more substances capable of up-regulating or down-regulating the Notch signalling pathway are then typically added to the culture medium together with the antigen of interest. The antigen may be added before, after or at substantially the same time as the substance(s). Cells are typically incubated with the substance(s) and antigen for at least one hour, preferably at least 3 hours, at 37° C. If required, a small aliquot of cells may be tested for modulated target gene expression as described above. Alternatively, cell activity may be measured by the inhibition of T cell activation by monitoring surface markers, cytokine secretion or proliferation as described in WO98/20142. APCs transfected with a nucleic acid construct directing the expression of, for example Serrate, may be used as a control.

As discussed above, polypeptide substances may be administered to APCs by introducing nucleic acid constructs/viral vectors encoding the polypeptide into cells under conditions that allow for expression of the polypeptide in the APC. Similarly, nucleic acid constructs encoding antigens may be introduced into the APCs by transfection, viral infection or viral transduction. The resulting APCs that show increased levels of Notch signalling are now ready for use.

The techniques described below are described in relation to T cells, but are equally applicable to B cells. The techniques employed are essentially identical to that described for APCs alone except that T cells are generally co-cultured with the APCs. However, it may be preferred to prepare primed APCs first and then incubate them with T cells. For example, once the primed APCs have been prepared, they may be pelleted and washed with PBS before being resuspended in fresh culture medium. Alternatively, the T cell may be incubated with a first substance (or set of substances) to modulate Notch signalling, washed, resuspended and then incubated with the primed APC in the absence of both the substance(s) used to modulate the APC and the substance(s) used to modulate the T cell. Alternatively, T cells may be cultured and primed in the absence of APCs by use of APC substitutes such as anti-TCR antibodies (e.g. anti-CD3) with or without antibodies to costimulatory molecules (e.g. anti-CD28) or alternatively T cells may be activated with MHC-peptide complexes (e.g. tetramers).

Incubations will typically be for at least 1 hour, preferably at least 3 or 6 hours, in suitable culture medium at 37° C. Modification of immune responses/tolerance may be determined by subsequently challenging T cells with antigen and measuring cytokine (eg IL-2) production compared with control cells not exposed to APCs.

T cells or B cells which have been primed in this way may be used according to the invention to modify immune responses/tolerance in other T cells or B cells.

Therapeutic Uses

A. Immunological Uses of the Present Invention

In a preferred embodiment, the constructs of the present invention may be used to modify immune responses in the immune system of a mammal, such as a human. Preferably such modulation of the immune system is effected by control of immune cell, preferably T-cell, preferably peripheral T-cell, activity.

A detailed description of the Notch signalling pathway and conditions affected by it may be found in our WO98/20142, WO00/36089 and PCT/GB00/04391.

Diseased or infectious states that may be described as being mediated by T cells include, but are not limited to, any one or more of asthma, allergy, graft rejection, autoimmunity, tumour induced aberrations to the T cell system and infectious diseases such as those caused by Plasmodium species, Microfilariae, Helminths, Mycobacteria, HIV, Cytomegalovirus, Pseudomonas, Toxoplasma, Echinococcus, Haemophilus influenza type B, measles, Hepatitis C or Toxicara. Thus particular conditions that may be treated or prevented which are mediated by T cells include multiple schlerosis, rheumatoid arthritis and diabetes. The present invention may also be used in organ transplantation or bone marrow transplantation.

As indicated above, the present invention is useful in treating immune disorders such as autoimmune diseases or graft rejection such as allograft rejection.

Autoimmune Disease

Examples of disorders that may be treated include a group commonly called autoimmune diseases. The spectrum of autoimmune disorders ranges from organ specific diseases (such as thyroiditis, insulitis, multiple sclerosis, iridocyclitis, uveitis, orchitis, hepatitis, Addison's disease, myasthenia gravis) to systemic illnesses such as rheumatoid arthritis or lupus erythematosus. Other disorders include immune hyperreactivity, such as allergic reactions. In more detail: Organ-specific autoimmune diseases include multiple sclerosis, insulin dependent diabetes mellitus, several forms of anemia (aplastic, hemolytic), autoimmune hepatitis, thyroiditis, insulitis, iridocyclitis, scleritis, uveitis, orchitis, myasthenia gravis, idiopathic thrombocytopenic purpura, inflammatory bowel diseases (Crohn's disease, ulcerative colitis).

Systemic autoimmune diseases include: rheumatoid arthritis, juvenile arthritis, scleroderma and systemic sclerosis, sjogren's syndrom, undifferentiated connective tissue syndrome, antiphospholipid syndrome, different forms of vasculitis (polyarteritis nodosa, allergic granulomatosis and angiitis, Wegner's granulomatosis, Kawasaki disease, hypersensitivity vasculitis, Henoch-Schoenlein purpura, Behcet's Syndrome, Takayasu arteritis, Giant cell arteritis, Thrombangiitis obliterans), lupus erythematosus, polymyalgia rheumatica, essentiell (mixed) cryoglobulinemia, Psoriasis vulgaris and psoriatic arthritis, diffus fasciitis with or without eosinophilia, polymyositis and other idiopathic inflammatory myopathies, relapsing panniculitis, relapsing polychondritis, lymphomatoid granulomatosis, erythema nodosum, ankylosing spondylitis, Reiter's syndrome, different forms of inflammatory dermatitis.

A more extensive list of disorders includes: unwanted immune reactions and inflammation including arthritis, including rheumatoid arthritis, inflammation associated with hypersensitivity, allergic reactions, asthma, systemic lupus erythematosus, collagen diseases and other autoimmune diseases, inflammation associated with atherosclerosis, arteriosclerosis, atherosclerotic heart disease, reperfusion injury, cardiac arrest, myocardial infarction, vascular inflammatory disorders, respiratory distress syndrome or other cardiopulmonary diseases, inflammation associated with peptic ulcer, ulcerative colitis and other diseases of the gastrointestinal tract, hepatic fibrosis, liver cirrhosis or other hepatic diseases, thyroiditis or other glandular diseases, glomerulonephritis or other renal and urologic diseases, otitis or other oto-rhino-laryngological diseases, dermatitis or other dermal diseases, periodontal diseases or other dental diseases, orchitis or epididimo-orchitis, infertility, orchidal trauma or other immune-related testicular diseases, placental dysfunction, placental insufficiency, habitual abortion, eclampsia, pre-eclampsia and other immune and/or inflammatory-related gynaecological diseases, posterior uveitis, intermediate uveitis, anterior uveitis, conjunctivitis, chorioretinitis, uveoretinitis, optic neuritis, intraocular inflammation, e.g. retinitis or cystoid macular oedema, sympathetic ophthalmia, scleritis, retinitis pigmentosa, immune and inflammatory components of degenerative fondus disease, inflammatory components of ocular trauma, ocular inflammation caused by infection, proliferative vitreo-retinopathies, acute ischaemic optic neuropathy, excessive scarring, e.g. following glaucoma filtration operation, immune and/or inflammation reaction against ocular implants and other immune and inflammatory-related ophthalmic diseases, inflammation associated with autoimmune diseases or conditions or disorders where, both in the central nervous system (CNS) or in any other organ, immune and/or inflammation suppression would be beneficial, Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, inflammatory components of stokes, post-polio syndrome, immune and inflammatory components of psychiatric disorders, myelitis, encephalitis, subacute sclerosing pan-encephalitis, encephalomyelitis, acute neuropathy, subacute neuropathy, chronic neuropathy, Guillaim-Barre syndrome, Sydenham chora, myasthenia gravis, pseudo-tumor cerebri, Down's Syndrome, Huntington's disease, amyotrophic lateral sclerosis, inflammatory components of CNS compression or CNS trauma or infections of the CNS, inflammatory components of muscular atrophies and dystrophies, and immune and inflammatory related diseases, conditions or disorders of the central and peripheral nervous systems, post-traumatic inflammation, septic shock, infectious diseases, inflammatory complications or side effects of surgery or organ, inflammatory and/or immune complications and side effects of gene therapy, e.g. due to infection with a viral carrier, or inflammation associated with AIDS, to suppress or inhibit a humoral and/or cellular immune response, to treat or ameliorate monocyte or leukocyte proliferative diseases, e.g. leukaemia, by reducing the amount of monocytes or lymphocytes, for the prevention and/or treatment of graft rejection in cases of transplantation of natural or artificial cells, tissue and organs such as cornea, bone marrow, organs, lenses, pacemakers, natural or artificial skin tissue.

Transplant Rejection

The present invention may be used, for example, for the treatment of organ transplants (e.g. kidney, heart, lung, liver or pancreas transplants), tissue transplants (e.g. skin grafts) or cell transplants (e.g. bone marrow transplants or blood transfusions).

A brief overview of the most common types of organ and tissue transplants is set out below.

i) Kidney Transplants:

Kidneys are the most commonly transplanted organs. Kidneys can be donated by both cadavers and living donors and kidney transplants can be used to treat numerous clinical indications (including diabetes, various types of nephritis and kidney failure). Surgical procedure for kidney transplantation is relatively simple. However, matching blood types and histocompatibility groups is desirable to avoid graft rejection. It is indeed important that a graft is accepted as many patients can become “sensitised” after rejecting a first transplant. Sensitisation results in the formation of antibodies and the activation of cellular mechanisms directed against kidney antigens. Thus, any subsequent graft containing antigens in common with the first is likely to be rejected. As a result, many kidney transplant patients must remain on some form of immunosuppressive treatment for the rest of their lives, giving rise to complications such as infection and metabolic bone disease.

ii) Heart Transplantation

Heart transplantation is a very complex and high-risk procedure. Donor hearts must be maintained in such a manner that they will begin beating when they are placed in the recipient and can therefore only be kept viable for a limited period under very specific conditions. They can also only be taken from brain-dead donors. Heart transplants can be used to treat various types of heart disease and/or damage. HLA matching is obviously desirable but often impossible because of the limited supply of hearts and the urgency of the procedure.

iii) Lung Transplantation

Lung transplantation is used (either by itself or in combination with heart transplantation) to treat diseases such as cystic fibrosis and acute damage to the lungs (e.g. caused by smoke inhalation). Lungs for use in transplants are normally recovered from brain-dead donors.

iv) Pancreas Transplantation

Pancreas transplantation is mainly used to treat diabetes mellitus, a disease caused by malfunction of insulin-producing islet cells in the pancreas. Organs for transplantation can only be recovered from cadavers although it should be noted that transplantation of the complete pancreas is not necessary to restore the function needed to produce insulin in a controlled fashion. Indeed, transplantation of the islet cells alone could be sufficient. Because kidney failure is a frequent complication of advanced diabetes, kidney and pancreas transplants are often carried out simultaneously.

v) Skin Grafting

Most skin transplants are done with autologous tissue. However, in cases of severe burning (for example), grafts of foreign tissue may be required (although it should be noted that these grafts are generally used as biological dressings as the graft will not grow on the host and will have to be replaced at regular intervals). In cases of true allogenic skin grafting, rejection may be prevented by the use of immunosuppressive therapy. However, this leads to an increased risk of infection and is therefore a major drawback in burn victims.

vi) Liver Transplantation

Liver transplants are used to treat organ damage caused by viral diseases such as hepititis, or by exposure to harmful chemicals (e.g. by chronic alcoholism). Liver transplants are also used to treat congenital abnormalities. The liver is a large and complicated organ meaning that transplantation initially posed a technical problem. However, most transplants (65%) now survive for more than a year and it has been found that a liver from a single donor may be split and given to two recipients. Although there is a relatively low rate of graft rejection by liver transplant patients, leukocytes within the donor organ together with anti-blood group antibodies can mediate antibody-dependent hemolysis of recipient red blood cells if there is a mismatch of blood groups. In addition, manifestations of GVHD have occurred in liver transplants even when donor and recipient are blood-group compatible.

Vaccines and Cancer Vaccines

The constructs of the present invention may also be used in vaccine compositions such as cancer and pathogen vaccines.

Vaccine Compositions

Conjugates according to the present invention which inhibit Notch signalling may be employed in vaccine compositions (such as pathogen or cancer vaccines) to protect or treat a mammal susceptible to, or suffering from disease, by means of administering said vaccine via a mucosal route, such as the oral/bucal/intestinal/vaginal/rectal or nasal route. Such administration may for example be in a droplet, spray, or dry powdered form. Nebulised or aerosolised vaccine formulations may also be used where appropriate.

Enteric formulations such as gastro resistant capsules and granules for oral administration, suppositories for rectal or vaginal administration may also be used. The present invention may also be used to enhance the immunogenicity of antigens applied to the skin, for example by intradermal, transdermal or transcutaneous delivery. In addition, the adjuvants of the present invention may be parentally delivered, for example by intramuscular or subcutaneous administration.

Depending on the route of administration, a variety of administration devices may be used. For example, for intranasal administration a spray device such as the commercially available Accuspray (Becton Dickinson) may be used.

Preferred spray devices for intranasal use are devices for which the performance of the device is not dependent upon the pressure applied by the user. These devices are known as pressure threshold devices. Liquid is released from the nozzle only when a threshold pressure is attained. These devices make it easier to achieve a spray with a regular droplet size. Pressure threshold devices suitable for use with the present invention are known in the art and are described for example in WO 91/13281 and EP 311 863 B. Such devices are commercially available from Pfeiffer GmbH.

For certain vaccine formulations, other vaccine components may be included in the formulation. For example the adjuvant formulations of the present invention may also comprise a bile acid or derivative of cholic acid. Suitably the derivative of cholic acid is a salt thereof, for example a sodium salt thereof. Examples of bile acids include cholic acid itself, deoxycholic acid, chenodeoxy colic acid, lithocholic acid, taurodeoxycholate ursodeoxycholic acid, hyodeoxycholic acid and derivatives like glyco-, tauro-, amidopropyl-1-propanesulfonic- and amidopropyl-2-hydroxy-1-propanesulfonic-derivatives of the above bile acids, or N,N-bis(3DGluconoamidopropyl)deoxycholamide.

Suitably, an adjuvant formulation of the present invention may be in the form of an aqueous solution or a suspension of non-vesicular forms. Such formulations are convenient to manufacture, and also to sterilise (for example by terminal filtration through a 450 or 220 nm pore membrane).

Suitably, the route of administration may be via the skin, intramuscular or via a mucosal surface such as the nasal mucosa. When the admixture is administered via the nasal mucosa, the admixture may for example be administered as a spray. The methods to enhance an immune response may be either a priming or boosting dose of the vaccine.

The term “adjuvant” as used herein includes an agent having the ability to enhance the immune response of a vertebrate subject's immune system to an antigen or antigenic determinant.

The term “immune response” includes any response to an antigen or antigenic determinant by the immune system of a subject. Immune responses include for example humoral immune responses (e. g. production of antigen-specific antibodies) and cell-mediated immune responses (e. g. lymphocyte proliferation).

The term “cell-mediated immune response” includes the immunological defence provided by lymphocytes, such as the defence provided by T cell lymphocytes when they come into close proximity with their victim cells.

When “lymphocyte proliferation” is measured, the ability of lymphocytes to proliferate in response to specific antigen may be measured. Lymphocyte proliferation includes B cell, T-helper cell or CTL cell proliferation.

Compositions of the present invention may be used to formulate vaccines containing antigens derived from a wide variety of sources. For example, antigens may include human, bacterial, or viral nucleic acid, pathogen derived antigen or antigenic preparations, host-derived antigens, including GnRH and IgE peptides, recombinantly produced protein or peptides, and chimeric fusion proteins.

Preferably the vaccine formulations of the present invention contain an antigen or antigenic composition capable of eliciting an immune response against a human pathogen. The antigen or antigens may, for example, be peptides/proteins, polysaccharides and lipids and may be derived from pathogens such as viruses, bacteria and parasites/fungi as follows:

Viral Antigens

Viral antigens or antigenic determinants may be derived, for example, from:

Cytomegalovirus (especially Human, such as gB or derivatives thereof); Epstein Barr virus (such as gp350); flaviviruses (e.g. Yellow Fever Virus, Dengue Virus, Tick-borne encephalitis virus, Japanese Encephalitis Virus); hepatitis virus such as hepatitis B virus (for example Hepatitis B Surface antigen such as the PreS1, PreS2 and S antigens described in EP-A-414 374; EP-A-0304 578, and EP-A-198474), hepatitis A virus, hepatitis C virus and hepatitis E virus; HIV-1, (such as tat, nef, gp120 or gp160); human herpes viruses, such as gD or derivatives thereof or Immediate Early protein such as ICP27 from HSV1 or HSV2; human papilloma viruses (for example HPV6, 11, 16, 18); Influenza virus (whole live or inactivated virus, split influenza virus, grown in eggs or MDCK cells, or Vero cells or whole flu virosomes (as described by Gluck, Vaccine, 1992,10, 915-920) or purified or recombinant proteins thereof, such as NP, NA, HA, or M proteins); measles virus; mumps virus; parainfluenza virus; rabies virus; Respiratory Syncytial virus (such as F and G proteins); rotavirus (including live attenuated viruses); smallpox virus; Varicella Zoster Virus (such as gpI, II and IE63); and the HPV viruses responsible for cervical cancer (for example the early proteins E6 or E7 in fusion with a protein D carrier to form Protein D-E6 or E7 fusions from HPV 16, or combinations thereof; or combinations of E6 or E7 with L2 (see for example WO 96/26277).

Bacterial Antigens

Bacterial antigens or antigenic determinants may be derived, for example, from:

Bacillus spp., including B. anthracis (e.g. botulinum toxin); Bordetella spp, including B. pertussis (for example pertactin, pertussis toxin, filamenteous hemagglutinin, adenylate cyclase, fimbriae); Borrelia spp., including B. burgdorferi (e.g. OspA, OspC, DbpA, DbpB), B. garinii (e.g. OspA, OspC, DbpA, DbpB), B. afzelii (e.g. OspA, OspC, DbpA, DbpB), B. andersonii (e.g. OspA, OspC, DbpA, DbpB), B. hermsii; Campylobacter spp, including C. jejuni (for example toxins, adhesins and invasins) and C. coli;

Chlamydia spp., including C. trachomatis (e.g. MOMP, heparin-binding proteins), C. pneumonie (e.g. MOMP, heparin-binding proteins), C. psittaci; Clostridium spp., including C. tetani (such as tetanus toxin), C. botulinum (for example botulinum toxin), C. difficile (e.g. clostridium toxins A or B); Corynebacterium spp., including C. diphtheriae (e.g. diphtheria toxin); Ehrlichia spp., including E. equi and the agent of the Human Granulocytic Ehrlichiosis; Rickettsia spp, including R. rickettsii;

Enterococcus spp., including E. faecalis, E. faecium; Escherichia spp, including enterotoxic E. coli (for example colonization factors, heat-labile toxin or derivatives thereof, or heat-stable toxin), enterohemorragic E. coli, enteropathogenic E. coli (for example shiga toxin-like toxin); Haemophilus spp., including H. influenzae type B (eg PRP), non-typable H. influenzae, for example OMP26, high molecular weight adhesins, P5, P6, protein D and lipoprotein D, and fimbrin and fimbrin derived peptides (see for example U.S. Pat. No. 5,843,464); Helicobacter spp, including H. pylori (for example urease, catalase, vacuolating toxin); Pseudomonas spp, including P. aeruginosa;

Legionella spp, including L. pneumophila; Leptospira spp., including L. interrogans; Listeria spp., including L. monocytogenes; Moraxella spp, including M catarrhalis, also known as Branhamella catarrhalis (for example high and low molecular weight adhesins and invasins); Morexella Catarrhalis (including outer membrane vesicles thereof, and OMP106 (see for example W097/41731)); Mycobacterium spp., including M. tuberculosis (for example ESAT6, Antigen 85A, —B or —C), M. bovis, M. leprae, M. avium, M. paratuberculosis, M. smegmatis; Neisseria spp, including N. gonorrhea and N. meningitidis (for example capsular polysaccharides and conjugates thereof, transferrin-binding proteins, lactoferrin binding proteins, PilC, adhesins); Neisseria mengitidis B (including outer membrane vesicles thereof, and NspA (see for example WO 96/29412); Salmonella spp, including S. typhi, S. paratyphi, S. choleraesuis, S. enteritidis; Shigella spp, including S. sonnei, S. dysenteriae, S. flexnerii; Staphylococcus spp., including S. aureus, S. epidermidis; Streptococcus spp, including S. pneumonie (e.g. capsular polysaccharides and conjugates thereof, PsaA, PspA, streptolysin, choline-binding proteins) and the protein antigen Pneumolysin (Biochem Biophys Acta, 1989,67,1007; Rubins et al., Microbial Pathogenesis, 25,337-342), and mutant detoxified derivatives thereof (see for example WO 90/06951; WO 99/03884); Treponema spp., including T. pallidum (e.g. the outer membrane proteins), T. denticola, T. hyodysenteriae; Vibrio spp, including V. cholera (for example cholera toxin); and Yersinia spp, including Y. enterocolitica (for example a Yop protein), Y. pestis, Y. pseudotuberculosis.

Parasite/Fungal Antigens

Parasitic/fungal antigens or antigenic determinants may be derived, for example, from:

Babesia spp., including B. microti; Candida spp., including C. albicans; Cryptococcus spp., including C. neoformans; Entamoeba spp., including E. histolytica; Giardia spp., including G. lamblia; Leshmania spp., including L. major; Plasmodium faciparum (MSP1, AMA1, MSP3, EBA, GLURP, RAP1, RAP2, Sequestrin, PfEMP1, Pf332, LSA1, LSA3, STARP, SALSA, PfEXP1, Pfs25, Pfs28, PFS27/25, Pfsl6, Pfs48/45, Pfs230 and their analogues in Plasmodium spp.);

Pneumocystis spp., including P. carinii; Schisostoma spp., including S. mansoni; Trichomonas spp., including T. vaginalis; Toxoplasma spp., including T. gondii (for example SAG2, SAG3, Tg34); Trypanosoma spp., including T. cruzi.

Approved/licensed vaccines include, for example anthrax vaccines such as Biothrax (BioPort Corp); tuberculosis (BCG) vaccines such as TICE BCG (Organon Teknika Corp) and Mycobax (Aventis Pasteur, Ltd); diphtheria & tetanus toxoid and acellular pertussis (DTP) vaccines such as Tripedia (Aventis Pasteur, Inc), Infanrix (GlaxoSmithKline), and DAPTACEL (Aventis Pasteur, Ltd); Haemophilus b conjugate vaccines (eg diphtheria CRM197 protein conjugates such as HibTITER from Lederle Lab Div, American Cyanamid Co; meningococcal protein conjugates such as PedvaxHIB from Merck & Co, Inc; and tetanus toxoid conjugates such as ActHIB from Aventis Pasteur, SA); Hepatitis A vaccines such as Havrix (GlaxoSmithKline) and VAQTA (Merck & Co, Inc); combined Hepatitis A and Hepatitis B (recombinant) vaccines such as Twinrix (GlaxoSmithKline); recombinant Hepatitis B vaccines such as Recombivax HB (Merck & Co, Inc) and Engerix-B (GlaxoSmithKline); influenza virus vaccines such as Fluvirin (Evans Vaccine), FluShield (Wyeth Laboratories, Inc) and Fluzone (Aventis Pasteur, Inc); Japanese Encephalitis virus vaccine such as JE-Vax (Research Foundation for Microbial Diseases of Osaka University); Measles virus vaccines such as Attenuvax (Merck & Co, Inc); measles and mumps virus vaccines such as M-M-Vax (Merck & Co, Inc); measles, mumps, and rubella virus vaccines such as M-M-R II (Merck & Co, Inc); meningococcal polysaccharide vaccines (Groups A, C, Y and W-135 combined) such as Menomune-A/C/Y/W-135 (Aventis Pasteur, Inc); mumps virus vaccines such as Mumpsvax (Merck & Co, Inc); pneumococcal vaccines such as Pneumovax (Merck & Co, Inc) and Pnu-Imune (Lederle Lab Div, American Cyanamid Co); Pneumococcal 7-valent conjugate vaccines (eg diphtheria CRM197 Protein conjugates such as Prevnar from Lederle Lab Div, American Cyanamid Co); poliovirus vaccines such as Poliovax (Aventis Pasteur, Ltd); poliovirus vaccines such as IPOL (Aventis Pasteur, SA); rabies vaccines such as Imovax (Aventis Pasteur, SA) and RabAvert (Chiron Behring GmbH & Co); rubella virus vaccines such as Meruvax II (Merck & Co, Inc); Typhoid Vi polysaccharide vaccines such as TYPHIM Vi (Aventis Pasteur, SA); Varicella virus vaccines such as Varivax (Merck & Co, Inc) and Yellow Fever vaccines such as YF-Vax (Aventis Pasteur, Inc).

Cancer/Tumour Antigens

The term “cancer antigen or antigenic determinant” or “tumour antigen or antigenic determinant” as used herein preferably means an antigen or antigenic determinant which is present on (or associated with) a cancer cell and not typically on normal cells, or an antigen or antigenic determinant which is present on cancer cells in greater amounts than on normal (non-cancer) cells, or an antigen or antigenic determinant which is present on cancer cells in a different form than that found on normal (non-cancer) cells.

Cancer antigens include, for example (but without limitation):

beta chain of human chorionic gonadotropin (hCG beta) antigen, carcinoembryonic antigen, EGFRvIII antigen, Globo H antigen, GM2 antigen, GP100 antigen, HER2/neu antigen, KSA antigen, Le (y) antigen, MUCI antigen, MAGE 1 antigen, MAGE 2 antigen, MUC2 antigen, MUC3 antigen, MUC4 antigen, MUC5AC antigen, MUC5B antigen, MUC7 antigen, PSA antigen, PSCA antigen, PSMA antigen,

Thompson-Friedenreich antigen (TF), Tn antigen, sTn antigen, TRP 1 antigen, TRP 2 antigen, tumor-specific immunoglobulin variable region and tyrosinase antigen.

It will be appreciated that in accordance with this aspect of the present invention antigens and antigenic determinants may be used in many different forms. For example, antigens or antigenic determinants may be present as isolated proteins or peptides (for example in so-called “subunit vaccines”) or, for example, as cell-associated or virus-associated antigens or antigenic determinants (for example in either live or killed pathogen strains). Live pathogens will preferably be attenuated in known manner. Alternatively, antigens or antigenic determinants may be generated in situ in the subject by use of a polynucleotide coding for an antigen or antigenic determinant (as in so-called “DNA vaccination”, although it will be appreciated that the polynucleotides which may be used with this approach are not limited to DNA, and may also include RNA and modified polynucleotides as discussed above).

B. Non-immunological Uses of the Present Invention

Cell Fate/Cancer Indications

It will be appreciated however that the constructs of the present invention, as modulators of Notch sigalling, may also be used for altering the fate of a cell, tissue or organ type by altering Notch pathway function in a cell by a partially or fully non-immunological mode of action (e.g. by modifying general cell fate, differentiation or proliferation), as described, for example in WO 92/07474, WO 96/27610, WO 97/01571, U.S. Pat. No. 5,648,464, U.S. Pat. No. 5,849,869 and U.S. Pat. No. 6,004,924 (Yale University/Imperial Cancer Technology), the texts of which are herein incorporated by reference.

Thus, the conjugates of the present invention are also useful in methods for altering the fate of any cell, tissue or organ type by altering Notch pathway function in the cell. Thus, for example, the present constructs also have application in the treatment of malignant and pre-neoplastic disorders for example by an antiproliferative, rather than immunological mechanism. For example, in the cancer field the conjugates of the present invention are especially useful in relation to adenocarcinomas such as: small cell lung cancer, and cancer of the kidney, uterus, prostrate, bladder, ovary, colon and breast. For example, malignancies which may be treatable according to the present invention include acute and chronic leukemias, lymphomas, myelomas, sarcomas such as Fibrosarcoma, myxosarcoma, liposarcoma, lymphangioendotheliosarcoma, angiosarcoma, endotheliosarcoma, chondrosarcoma, osteogenic sarcoma, chordoma, lymphangiosarcoma, synovioma, mesothelioma, leimyosarcoma, rhabdomyosarcoma, colon carcinoma, ovarian cancer, prostate cancer, pancreatic cancer, breasy cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, choriocarcinoma, renal cell carcinoma, hepatoma, bile duct carcinoma seminoma, embryonal carcinoma, cervical cancer, testicular tumour, lung carcinoma, small cell lung carcinoma, bladder carcinoma, epithelial carcinoma, glioma, astrocytoma, ependymoma, pinealoma, hemangioblastoma, acoustic neuoma, medulloblastoma, craniopharyngioma, oligodendroglioma, menangioma, melanoma, neutroblastoma and retinoblastoma.

The present invention may also have application in the treatment of nervous system disorders. Nervous system disorders which may be treated according to the present invention include neurological lesions including traumatic lesions resulting from physical injuries; ischaemic lesions; malignant lesions; infectious lesions such as those caused by HIV, herpes zoster or herpes simplex virus, Lyme disease, tuberculosis or syphilis; degenerative lesions and diseases and demyelinated lesions.

The present invention may be used to treat, for example, diabetes (including diabetic neuropathy, Bell's palsy), systemic lupus erythematosus, sarcoidosis, multiple sclerosis, human immunodeficiency virus-associated myelopathy, transverse myelopathy or various etiologies, progressive multifocal leukoencephalopathy, central pontine myelinolysis, Parkinson's disease, Alzheimer's disease, Huntington's chorea, amyotrophic lateral sclerosis, cerebral infarction or ischemia, spinal cord infarction or ischemia, progressive spinal muscular atrophy, progressive bulbar palsy, primary lateral sclerosis, infantile and juvenile muscular atrophy, progressive bulbar paralysis of childhood (Fazio-Londe syndrome), poliomyelitis and the post polio syndrome, and Hereditary Motorsensory Neuropathy (Charcot-Marie-Tooth Disease).

The present invention may further be useful in the promotion of tissue regeneration and repair, for example by modification of differentiation processes. The present invention, therefore, may also be used to treat diseases associated with defective tissue repair and regeneration such as, for example, cirrhosis of the liver, hypertrophic scar formation and psoriasis. The invention may also be useful in the treatment of neutropenia or anemia and in techniques of organ regeneration and tissue engineering and stem cell treatments.

Pharmaceutical Compositions

Preferably the active agents (eg conjugates and constructs) of the present invention are administered in the form of pharmaceutical compositions. The pharmaceutical compositions may be for human or animal usage in human and veterinary medicine and in addition to one or more active agents will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s). Preservatives, stabilizers, dyes and even flavoring agents may also be provided in such a pharmaceutical composition. Examples of preservatives include sodium benzoate, sorbic acid and esters of p-hydroxybenzoic acid. Antioxidants and suspending agents may be also used.

Administration

Typically, a physician will determine the actual dosage which will be most suitable for an individual subject and it will vary with the age, weight and response of the particular patient. The dosages below are exemplary of the average case. There can, of course, be individual instances where higher or lower dosage ranges are merited.

In one embodiment the therapeutic agents used in the present invention may be administered directly to patients in vivo. Alternatively or in addition, the agents may be administered to cells (such as T cells and/or APCs or stem or tissue cells) in an ex vivo manner. For example, leukocytes such as T cells or APCs may be obtained from a patient or donor in known manner, treated/incubated ex vivo in the manner of the present invention, and then administered to a patient.

In general, a therapeutically effective daily dose may for example range from 0.01 to 500 mg/kg, for example 0.01 to 50 mg/kg body weight of the subject to be treated, for example 0.1 to 20 mg/kg. The conjugate of the present invention may also be administered by intravenous infusion, at a dose which is likely to range from for example 0.001-10 mg/kg/hr.

A skilled practitioner will be able to determine readily the optimum route of administration and dosage for any particular patient depending on, for example, the age, weight and condition of the patient. Preferably the pharmaceutical compositions are in unit dosage form.

The agents of the present invention can be administered by any suitable means including, but not limited to, for example, oral, rectal, nasal, topical (including intradermal, transdermal, aerosol, buccal and sublingual), vaginal and parenteral (including subcutaneous, intramuscular, intravenous and intradermal) routes of administration.

Suitably the active agents are administered in combination with a pharmaceutically acceptable carrier or diluent as described under the heading “Pharmaceutical compositions” above. The pharmaceutically acceptable carrier or diluent may be, for example, sterile isotonic saline solutions, or other isotonic solutions such as phosphate-buffered saline. The conjugates of the present invention may suitably be admixed with any suitable binder(s), lubricant(s), suspending agent(s), coating agent(s), solubilising agent(s).

In one embodiment, it may be desired to formulate the compound in an orally active form. Thus, for some applications, active agents may be administered orally in the form of tablets containing excipients such as starch or lactose, or in capsules or ovules either alone or in admixture with excipients, or in the form of elixirs, solutions or suspensions containing flavouring or colouring agents. Doses such as tablets or capsules comprising the conjugates may be administered singly or two or more at a time, as appropriate. It is also possible to administer the conjugates in sustained release formulations.

Alternatively or in addition, active agents may be administered by inhalation, intranasally or in the form of aerosol, or in the form of a suppository or pessary, or they may be applied topically in the form of a lotion, solution, cream, ointment or dusting powder. An alternative means of transdermal administration is by use of a skin patch. For example, they can be incorporated into a cream consisting of an aqueous emulsion of polyethylene glycols or liquid paraffin. They can also be incorporated, for example at a concentration of between 1 and 10% by weight, into an ointment consisting of a white wax or white soft paraffin base together with such stabilisers and preservatives as may be required.

Active agents such as polynucleotides and proteins/polypeptides may also be administered by viral or non-viral techniques. Viral delivery mechanisms include but are not limited to adenoviral vectors, adeno-associated viral (AAV) vectors, herpes viral vectors, retroviral vectors, lentiviral vectors, and baculoviral vectors. Non-viral delivery mechanisms include lipid mediated transfection, liposomes, immunoliposomes, lipofectin, cationic facial amphiphiles (CFAs) and combinations thereof. The routes for such delivery mechanisms include, but are not limited to, mucosal, nasal, oral, parenteral, gastrointestinal, topical, or sublingual routes. Active agents may also be adminstered by needleless systems, such as ballistic delivery on particles for delivery to the epidermis or dermis or other sites such as mucosal surfaces.

Active agents may also be injected parenterally, for example intracavernosally, intravenously, intramuscularly or subcutaneously

For parenteral administration, active agents may for example be used in the form of a sterile aqueous solution which may contain other substances, for example enough salts or monosaccharides to make the solution isotonic with blood.

For buccal or sublingual administration, agents may for example be administered in the form of tablets or lozenges which can be formulated in a conventional manner.

For oral, parenteral, buccal and sublingual administration to subjects (such as patients), the dosage level of active agents and their pharmaceutically acceptable salts and solvates may typically be from 10 to 500 mg (in single or divided doses). Thus, and by way of example, tablets or capsules may contain from 5 to 100 mg of active agent for administration singly, or two or more at a time, as appropriate. As indicated above, the physician will determine the actual dosage which will be most suitable for an individual patient and it will vary with the age, weight and response of the particular patient. It is to be noted that whilst the above-mentioned dosages are exemplary of the average case there can, of course, be individual instances where higher or lower dosage ranges are merited and such dose ranges are within the scope of this invention.

The routes of administration and dosages described are intended only as a guide since a skilled practitioner will be able to determine readily the optimum route of administration and dosage for any particular patient depending on, for example, the age, weight and condition of the patient.

The term treatment or therapy as used herein should be taken to encompass diagnostic and prophylatic applications.

The treatment of the present invention includes both human and veterinary applications.

The active agents of the present invention may also be administered with other active agents such as, for example, immunosuppressants, steroids or anticancer agents.

Where treated ex-vivo, modified cells of the present invention are preferably administered to a host by direct injection into the lymph nodes of the patient. Typically from 10⁴ to 10⁸ treated cells, preferably from 10⁵ to 10⁷ cells, more preferably about 10⁶ cells are administered to the patient. Preferably, the cells will be taken from an enriched cell population.

As used herein, the term “enriched” as applied to the cell populations of the invention refers to a more homogeneous population of cells which have fewer other cells with which they are naturally associated. An enriched population of cells can be achieved by several methods known in the art. For example, an enriched population of T-cells can be obtained using immunoaffinity chromatography using monoclonal antibodies specific for determinants found only on T-cells.

Enriched populations can also be obtained from mixed cell suspensions by positive selection (collecting only the desired cells) or negative selection (removing the undesirable cells). The technology for capturing specific cells on affinity materials is well known in the art (Wigzel, et al., J. Exp. Med., 128:23, 1969; Mage, et al., J. Imnmunol. Meth., 15:47, 1977; Wysocki, et al., Proc. Natl. Acad. Sci. U.S.A., 75:2844, 1978; Schrempf-Decker, et al., J. Immunol Meth., 32:285, 1980; Muller-Sieburg, et al., Cell, 44:653, 1986).

Monoclonal antibodies against antigens specific for mature, differentiated cells have been used in a variety of negative selection strategies to remove undesired cells, for example, to deplete T-cells or malignant cells from allogeneic or autologous marrow grafts, respectively (Gee, et al., J.N.C.I. 80:154, 1988). Purification of human hematopoietic cells by negative selection with monoclonal antibodies and immunomagnetic microspheres can be accomplished using multiple monoclonal antibodies (Griffin, et al., Blood, 63:904, 1984).

Procedures for separation of cells may include magnetic separation, using antibodycoated magnetic beads, affinity chromatography, cytotoxic agents joined to a monoclonal antibody or used in conjunction with a monoclonal antibody, for example, complement and cytotoxins, and “panning” with antibodies attached to a solid matrix, for example, plate, or other convenient technique. Techniques providing accurate separation include fluorescence activated cell sorters, which can have varying degrees of sophistication, for example, a plurality of color channels, low angle and obtuse light scattering detecting channels, impedance channels, etc.

Combination Treatments

Combination treatments wherein active agents of the present invention are administered in combination with other active agents, antigens or antigenic determinants are also within the scope of the present invention.

By “simultaneously” is meant that the active agents are administered at substantially the same time, and preferably together in the same formulation.

By “contemporaneously” it is meant that the active agents are administered closely in time, e.g., one agent is administered within from about one minute to within about one day before or after another. Any contemporaneous time is useful. However, it will often be the case that when not administered simultaneously, the agents will be administered within about one minute to within about eight hours, and preferably within less than about one to about four hours. When administered contemporaneously, the agents are preferably administered at the same site on the animal. The term “same site” includes the exact location, but can be within about 0.5 to about 15 centimeters, preferably from within about 0.5 to about 5 centimeters.

The term “separately” as used herein means that the agents are administered at an interval, for example at an interval of about a day to several weeks or months. The active agents may be administered in either order.

The term “sequentially” as used herein means that the agents are administered in sequence, for example at an interval or intervals of minutes, hours, days or weeks. If appropriate the active agents may be administered in a regular repeating cycle.

It will be appreciated that in one embodiment the therapeutic agents used in the present invention may be administered directly to patients in vivo. Alternatively or in addition, the agents may be administered to cells such as T cells and/or APCs in an ex vivo manner. For example, leukocytes such as T cells or APCs may be obtained from a patient or donor in known manner, treated/incubated ex vivo in the manner of the present invention, and then administered to a patient. In addition, it will be appreciated that a combination of routes of administration may be employed if desired. For example, where appropriate one component (such as the modulator of Notch signalling) may be administered ex-vivo and the other may be administered in vivo, or vice versa.

Chemical Cross-Linking

Chemically coupled (cross-linked) sequences can be prepared from individual protein sequences and coupled using known chemical coupling techniques. A conjugate can for example be assembled using conventional solution- or solid-phase peptide synthesis methods, affording a fully protected precursor with only the terminal amino group in deprotected reactive form. This function can then be reacted directly with a protein for Notch signalling modulation or a suitable reactive derivative thereof. Alternatively, this amino group may be converted into a different functional group suitable for reaction with a cargo moiety or a linker. Thus, e.g. reaction of the amino group with succinic anhydride will provide a selectively addressable carboxyl group, while further peptide chain extension with a cysteine derivative will result in a selectively addressable thiol group. Once a suitable selectively addressable functional group has been obtained in the delivery vector precursor, a protein for Notch signalling modulation or a derivative thereof may be attached through e.g. amide, ester, or disulphide bond formation. Cross-linking reagents which can be utilized are discussed, for example, in Means, G. E. and Feeney, R. E., Chemical Modification of Proteins, Holden-Day, 1974, pp. 39-43.

As discussed above the polymer and proteins or polypeptides for Notch signalling modulation may be linked directly or indirectly suitably via a linker moiety. Direct linkage may occur through any convenient functional group on the protein for Notch signalling modulation such as a thiol, hydroxy, carboxy or amino group. Indirect linkage which is may sometimes be preferable, will occur through a linking moiety. Suitable linking moieties include bi- and multi-functional alkyl, aryl, aralkyl or peptidic moieties, alkyl, aryl or aralkyl aldehydes acids esters and anyhdrides, sulphydryl or carboxyl groups, such as maleimido benzoic acid derivatives, maleimido proprionic acid derivatives and succinimido derivatives or may be derived from cyanuric bromide or chloride, carbonyldiimidazole, succinimidyl esters or sulphonic halides and the like. The functional groups on the linker moiety used to form covalent bonds between linker and protein for Notch signalling modulation on the one hand, as well as linker and polymer on the other hand, may be two or more of, e.g., amino, hydrazino, hydroxyl, thiol, maleimido, carbonyl, and carboxyl groups, etc. The linker moiety may include a short sequence of eg from 1 to 4 amino acid residues that optionally includes a cysteine residue through which the linker moiety bonds to the target protein.

Modified/Humanised Antibodies

Preferably, antibodies for use to treat human patients will be chimeric or humanised antibodies. Antibody “humanisation” techniques are well known in the art. These techniques typically involve the use of recombinant DNA technology to manipulate DNA sequences encoding the polypeptide chains of the antibody molecule.

As described in U.S. Pat. No. 5,859,205 early methods for humanising monoclonal antibodies (Mabs) involved production of chimeric antibodies in which an antigen binding site comprising the complete variable domains of one antibody is linked to constant domains derived from another antibody. Such chimerisation procedures are described in EP-A-0120694 (Celltech Limited), EP-A-0125023 (Genentech Inc. and City of Hope), EP-A-0 171496 (Res. Dev. Corp. Japan), EP-A-0 173 494 (Stanford University), and WO 86/01533 (Celltech Limited). For example, WO 86/01533 discloses a process for preparing an antibody molecule having the variable domains from a mouse MAb and the constant domains from a human immunoglobulin.

In an alternative approach, described in EP-A-0239400 (Winter), the complementarity determining regions (CDRs) of a mouse MAb are grafted onto the framework regions of the variable domains of a human immunoglobulin by site directed mutagenesis using long oligonucleotides. Such CDR-grafted humanised antibodies are much less likely to give rise to an anti-antibody response than humanised chimeric antibodies in view of the much lower proportion of non-human amino acid sequence which they contain. Examples in which a mouse MAb recognising lysozyme and a rat MAb recognising an antigen on human T-cells were humanised by CDR-grafting have been described by Verhoeyen et al (Science, 239, 1534-1536, 1988) and Riechmann et al (Nature, 332, 323-324, 1988) respectively. The preparation of CDR-grafted antibody to the antigen on human T cells is also described in WO 89/07452 (Medical Research Council).

In WO 90/07861 Queen et al propose four criteria for designing humanised immunoglobulins. The first criterion is to use as the human acceptor the framework from a particular human immunoglobulin that is unusually homologous to the non-human donor immunoglobulin to be humanised, or to use a consensus framework from many human antibodies. The second criterion is to use the donor amino acid rather than the acceptor if the human acceptor residue is unusual and the donor residue is typical for human sequences at a specific residue of the framework. The third criterion is to use the donor framework amino acid residue rather than the acceptor at positions immediately adjacent to the CDRs. The fourth criterion is to use the donor amino acid residue at framework positions at which the amino acid is predicted to have a side chain atom within about 3 A of the CDRs in a three-dimensional immunoglobulin model and to be capable of interacting with the antigen or with the CDRs of the humanised immunoglobulin. It is proposed that criteria two, three or four may be applied in addition or alternatively to criterion one, and may be applied singly or in any combination.

Antigens and Allergens

In one embodiment, the conjugates of the present invention may be administered in simultaneous, separate or sequential combination with antigens or antigenic determinants (or polynucleotides coding therefor), to modify (increase or decrease) the immune response to such antigens or antigenic determinants.

An antigen suitable for use in the present invention may be any substance that can be recognised by the immune system, and is generally recognised by an antigen receptor. Preferably the antigen used in the present invention is an immunogen. An allergic response occurs when the host is re-exposed to an antigen that it has encountered previously.

The immune response to antigen is generally either cell mediated (T cell mediated killing) or humoral (antibody production via recognition of whole antigen). The pattern of cytokine production by TH cells involved in an immune response can influence which of these response types predominates: cell mediated immunity (TH1) is characterised by high IL-2 and IFNγ but low IL-4 production, whereas in humoral immunity (TH2) the pattern is low IL-2 and IFNγ but high IL-4, IL-5 and IL-13. Since the secretory pattern is modulated at the level of the secondary lymphoid organ or cells, then pharmacological manipulation of the specific TH cytokine pattern can influence the type and extent of the immune response generated.

The TH1-TH2 balance refers to the relative representation of the two different forms of helper T cells. The two forms have large scale and opposing effects on the immune system. If an immune response favours TH1 cells, then these cells will drive a cellular response, whereas TH2 cells will drive an antibody-dominated response. The type of antibodies responsible for some allergic reactions is induced by TH2 cells.

The antigen or allergen (or antigenic determinant thereof) used in the present invention may be a peptide, polypeptide, carbohydrate, protein, glycoprotein, or more complex material containing multiple antigenic epitopes such as a protein complex, cell-membrane preparation, whole cells (viable or non-viable cells), bacterial cells or virus/viral component. In particular, it is preferred to use antigens known to be associated with auto-immune diseases such as myelin basic protein (associated with multiple sclerosis), collagen (associated with rheumatoid arthritis), and insulin (diabetes), or antigens associated with rejection of non-self tissue such as MHC antigens or antigenic determinants thereof. Where primed the APCs and/or T cells of the present invention are to be used in tissue transplantation procedures, antigens may be obtained from the tissue donor. Polynucleotides coding for antigens or antigenic determinants which may be expessed in a subject may also be used.

The antigen or allergen moiety may for example be present as a derivative or complex, for example, a synthetic MHC-peptide complex i.e. a fragment of the MHC molecule bearing the antigen groove bearing an element of the antigen. Such complexes have been described in Altman et al., 1996.

Assays

Whether a substance can be used for modulating Notch-Notch ligand expression may be determined using suitable screening assays, for example, as described in our co-pending International Patent Application claiming priority from GB 0118153.6 (now published as WO 03/012441), or for example as described in the Examples herein.

For example, Notch signalling can be monitored either through protein assays or through nucleic acid assays. Activation of the Notch receptor leads to the proteolytic cleavage of its cytoplasmic domain and the translocation thereof into the cell nucleus. The “detectable signal” referred to herein may be any detectable manifestation attributable to the presence of the cleaved intracellular domain of Notch. Thus, increased Notch signalling can be assessed at the protein level by measuring intracellular concentrations of the cleaved Notch domain. Activation of the Notch receptor also catalyses a series of downstream reactions leading to changes in the levels of expression of certain well-defined genes. Thus, increased Notch signalling can be assessed at the nucleic acid level by say measuring intracellular concentrations of specific mRNAs. In one preferred embodiment of the present invention, the assay is a protein assay. In another preferred embodiment of the present invention, the assay is a nucleic acid assay.

The advantage of using a nucleic acid assay is that they are sensitive and that small samples can be analysed.

The intracellular concentration of a particular mRNA, measured at any given time, reflects the level of expression of the corresponding gene at that time. Thus, levels of mRNA of downstream target genes of the Notch signalling pathway can be measured in an indirect assay of the T-cells of the immune system. In particular, an increase in levels of Deltex, Hes-1 and/or IL-10 mRNA may, for instance, indicate induced anergy while an increase in levels of Dll-1 or IFN-γ mRNA, or in the levels of mRNA encoding cytokines such as IL-2, IL-5 and IL-1 3, may indicate improved responsiveness.

Various nucleic acid assays are known. Any convention technique which is known or which is subsequently disclosed may be employed. Examples of suitable nucleic acid assay are mentioned below and include amplification, PCR, RT-PCR, RNase protection, blotting, spectrometry, reporter gene assays, gene chip arrays and other hybridization methods.

In particular, gene presence, amplification and/or expression may be measured in a sample directly, for example, by conventional Southern blotting, Northern blotting to quantitate the transcription of mRNA, dot blotting (DNA or RNA analysis), or in situ hybridisation, using an appropriately labelled probe. Those skilled in the art will readily envisage how these methods may be modified, if desired.

PCR was originally developed as a means of amplifying DNA from an impure sample. The technique is based on a temperature cycle which repeatedly heats and cools the reaction solution allowing primers to anneal to target sequences and extension of those primers for the formation of duplicate daughter strands. RT-PCR uses an RNA template for generation of a first strand cDNA with a reverse transcriptase. The cDNA is then amplified according to standard PCR protocol. Repeated cycles of synthesis and denaturation result in an exponential increase in the number of copies of the target DNA produced. However, as reaction components become limiting, the rate of amplification decreases until a plateau is reached and there is little or no net increase in PCR product. The higher the starting copy number of the nucleic acid target, the sooner this “end-point” is reached.

Real-time PCR uses probes labeled with a fluorescent tag or fluorescent dyes and differs from end-point PCR for quantitative assays in that it is used to detect PCR products as they accumulate rather than for the measurement of product accumulation after a fixed number of cycles. The reactions are characterized by the point in time during cycling when amplification of a target sequence is first detected through a significant increase in fluorescence.

The ribonuclease protection (RNase protection) assay is an extremely sensitive technique for the quantitation of specific RNAs in solution. The ribonuclease protection assay can be performed on total cellular RNA or poly(A)-selected mRNA as a target. The sensitivity of the ribonuclease protection assay derives from the use of a complementary in vitro transcript probe which is radiolabeled to high specific activity. The probe and target RNA are hybridized in solution, after which the mixture is diluted and treated with ribonuclease (RNase) to degrade all remaining single-stranded RNA. The hybridized portion of the probe will be protected from digestion and can be visualized via electrophoresis of the mixture on a denaturing polyacrylamide gel followed by autoradiography. Since the protected fragments are analyzed by high resolution polyacrylamide gel electrophoresis, the ribonuclease protection assay can be employed to accurately map mRNA features. If the probe is hybridized at a molar excess with respect to the target RNA, then the resulting signal will be directly proportional to the amount of complementary RNA in the sample.

Gene expression may also be detected using a reporter system. Such a reporter system may comprise a readily identifiable marker under the control of an expression system, e.g. of the gene being monitored. Fluorescent markers, which can be detected and sorted by FACS, are preferred. Especially preferred are GFP and luciferase. Another type of preferred reporter is cell surface markers, i.e. proteins expressed on the cell surface and therefore easily identifiable.

In general, reporter constructs useful for detecting Notch signalling by expression of a reporter gene may be constructed according to the general teaching of Sambrook et al (1989). Typically, constructs according to the invention comprise a promoter by the gene of interest, and a coding sequence encoding the desired reporter constructs, for example of GFP or luciferase. Vectors encoding GFP and luciferase are known in the art and available commercially.

Sorting of cells, based upon detection of expression of genes, may be performed by any technique known in the art, as exemplified above. For example, cells may be sorted by flow cytometry or FACS. For a general reference, see Flow Cytometry and Cell Sorting: A Laboratory Manual (1992) A. Radbruch (Ed.), Springer Laboratory, New York.

Flow cytometry is a powerful method for studying and purifying cells. It has found wide application, particularly in immunology and cell biology: however, the capabilities of the FACS can be applied in many other fields of biology. The acronym F.A.C.S. stands for Fluorescence Activated Cell Sorting, and is used interchangeably with “flow cytometry”. The principle of FACS is that individual cells, held in a thin stream of fluid, are passed through one or more laser beams, causing light to be scattered and fluorescent dyes to emit light at various frequencies. Photomultiplier tubes (PMT) convert light to electrical signals, which are interpreted by software to generate data about the cells. Sub-populations of cells with defined characteristics can be identified and automatically sorted from the suspension at very high purity (˜100%).

FACS can be used to measure gene expression in cells transfected with recombinant DNA encoding polypeptides. This can be achieved directly, by labelling of the protein product, or indirectly by using a reporter gene in the construct. Examples of reporter genes are β-galactosidase and Green Fluorescent Protein (GFP). β-galactosidase activity can be detected by FACS using fluorogenic substrates such as fluorescein digalactoside (FDG). FDG is introduced into cells by hypotonic shock, and is cleaved by the enzyme to generate a fluorescent product, which is trapped within the cell. One enzyme can therefore generate a large amount of fluorescent product. Cells expressing GFP constructs will fluoresce without the addition of a substrate. Mutants of GFP are available which have different excitation frequencies, but which emit fluorescence in the same channel. In a two-laser FACS machine, it is possible to distinguish cells which are excited by the different lasers and therefore assay two transfections at the same time.

Alternative means of cell sorting may also be employed. For example, the invention comprises the use of nucleic acid probes complementary to mRNA. Such probes can be used to identify cells expressing polypeptides individually, such that they may subsequently be sorted either manually, or using FACS sorting. Nucleic acid probes complementary to mRNA may be prepared according to the teaching set forth above, using the general procedures as described by Sambrook et al (1989) supra.

In a preferred embodiment, the invention comprises the use of an antisense nucleic acid molecule, complementary to a mRNA, conjugated to a fluorophore which may be used in FACS cell sorting.

Methods have also been described for obtaining information about gene expression and identity using so-called gene chip arrays or high-density DNA arrays (Chee M. et al. (1996) Science 274:601-614 (Chee)). These high-density arrays are particularly useful for diagnostic and prognostic purposes. Use may also be made of In vivo Expression Technology (IVET) (Camilli et al. (1994) Proc Natl Acad Sci USA 91:2634-2638 (Camilli)). IVET identifies genes up-regulated during say treatment or disease when compared to laboratory culture.

The advantage of using a protein assay is that Notch activation can be directly measured. Assay techniques that can be used to determine levels of a polypeptide are well known to those skilled in the art. Such assay methods include radioimmunoassays, competitive-binding assays, Western Blot analysis, antibody sandwich assays, antibody detection, FACS and ELISA assays.

As described above the modulator of Notch signalling may also be an immune cell which has been treated to modulate expression or interaction of Notch, a Notch ligand or the Notch signalling pathway. Such cells may readily be prepared, for example, as described in WO 00/36089 in the name of Lorantis Ltd, the text of which is herein incorporated by reference.

Cells of the Immune System

Antigen Presenting Cells

Where required, antigen-presenting cells (APCs) may be “professional” antigen presenting cells or may be another cell that may be induced to present antigen to T cells. Alternatively a APC precursor may be used which differentiates or is activated under the conditions of culture to produce an APC. An APC for use in the ex vivo methods of the invention is typically isolated from a tumour or peripheral blood found within the body of a patient. Preferably the APC or precursor is of human origin. However, where APCs are used in preliminary in vitro screening procedures to identify and test suitable nucleic acid sequences, APCs from any suitable source, such as a healthy patient, may be used.

APCs include dendritic cells (DCs) such as interdigitating DCs or follicular DCs, Langerhans cells, PBMCs, macrophages, B-lymphocytes, or other cell types such as epithelial cells, fibroblasts or endothelial cells, activated or engineered by transfection to express a MHC molecule (Class I or II) on their surfaces. Precursors of APCs include CD34⁺ cells, monocytes, fibroblasts and endothelial cells. The APCs or precursors may be modified by the culture conditions or may be genetically modified, for instance by transfection of one or more genes encoding proteins which play a role in antigen presentation and/or in combination of selected cytokine genes which would promote to immune potentiation (for example IL-2, IL-12, IFN-γ, TNF-α, IL-18 etc.). Such proteins include MHC molecules (Class I or Class II), CD80, CD86, or CD40. Most preferably DCs or DC-precursors are included as a source of APCs.

Dendritic cells (DCs) can be isolated/prepared by a number of means, for example they can either be purified directly from peripheral blood, or generated from CD34⁺ precursor cells for example after mobilisation into peripheral blood by treatment with GM-CSF, or directly from bone marrow. From peripheral blood, adherent precursors can be treated with a GM-CSF/IL-4 mixture (Inaba K, et al. (1992) J. Exp. Med. 175: 1157-1167 (Inaba)), or from bone marrow, non-adherent CD34⁺ cells can be treated with GM-CSF and TNF-a (Caux C, et al. (1992) Nature 360: 258-261 (Caux)). DCs can also be routinely prepared from the peripheral blood of human volunteers, similarly to the method of Sallusto and Lanzavecchia (Sallusto F and Lanzavecchia A (1994) J. Exp. Med. 179: 1109-1118) using purified peripheral blood mononucleocytes (PBMCs) and treating 2 hour adherent cells with GM-CSF and IL-4. If required, these may be depleted of CD19⁺B cells and CD3⁺, CD2⁺T cells using magnetic beads (Coffin RS, et al. (1998) Gene Therapy 5: 718-722 (Coffin)). Culture conditions may include other cytokines such as GM-CSF or IL-4 for the maintenance and/or activity of the dendritic cells or other antigen presenting cells.

Thus, it will be understood that the term “antigen presenting cell or the like” as used herein is not intended to be limited to APCs. The skilled man will understand that any vehicle capable of presenting to the T cell population may be used, for the sake of convenience the term APCs is used to refer to all these. As indicated above, preferred examples of suitable APCs include dendritic cells, L cells, hybridomas, fibroblasts, lymphomas, macrophages, B cells or synthetic APCs such as lipid membranes.

T Cells

Where required, T cells from any suitable source, such as a healthy patient, may be used and may be obtained from blood or another source (such as lymph nodes, spleen, or bone marrow). They may optionally be enriched or purified by standard procedures. The T cells may be used in combination with other immune cells, obtained from the same or a different individual. Alternatively whole blood may be used or leukocyte enriched blood or purified white blood cells as a source of T cells and other cell types. It is particularly preferred to use helper T cells (CD4⁺). Alternatively other T cells such as CD8⁺ cells may be used. It may also be convenient to use cell lines such as T cell hybridomas.

Exposure of Agent to APCs and T Cells

T cells/APCs may be cultured as described above. The APCs/T cells may be incubated/exposed to substances which are capable of modulating Notch signalling. For example, they may be prepared for administration to a patient or incubated with T cells in vitro (ex vivo).

Introduction of Nucleic Acid Sequences into APCs and T-Cells

T-cells and APCs as described above may be cultured in a suitable culture medium such as DMEM or other defined media, optionally in the presence of fetal calf serum.

Polypeptide substances may be administered to T-cells and/or APCs by introducing nucleic acid constructs/viral vectors encoding the polypeptide into cells under conditions that allow for expression of the polypeptide in the T-cell and/or APC. Similarly, nucleic acid constructs encoding antisense constructs may be introduced into the T-cells and/or APCs by transfection, viral infection or viral transduction.

In a preferred embodiment, nucleotide sequences will be operably linked to control sequences, including promoters/enhancers and other expression regulation signals. The term “operably linked” means that the components described are in a relationship permitting them to function in their intended manner. A regulatory sequence “operably linked” to a coding sequence is peferably ligated in such a way that expression of the coding sequence is achieved under condition compatible with the control sequences.

The promoter is typically selected from promoters which are functional in mammalian cells, although prokaryotic promoters and promoters functional in other eukaryotic cells may be used. The promoter is typically derived from promoter sequences of viral or eukaryotic genes. For example, it may be a promoter derived from the genome of a cell in which expression is to occur. With respect to eukaryotic promoters, they may be promoters that function in a ubiquitous manner (such as promoters of a-actin, b-actin, tubulin) or, alternatively, a tissue-specific manner (such as promoters of the genes for pyruvate kinase). Tissue-specific promoters specific for lymphocytes, dendritic cells, skin, brain cells and epithelial cells within the eye are particularly preferred, for example the CD2, CD 11c, keratin 14, Wnt-1 and Rhodopsin promoters respectively. Preferably the epithelial cell promoter SPC is used. They may also be promoters that respond to specific stimuli, for example promoters that bind steroid hormone receptors. Viral promoters may also be used, for example the Moloney murine leukaemia virus long terminal repeat (MMLV LTR) promoter, the rous sarcoma virus (RSV) LTR promoter or the human cytomegalovirus (CMV) IE promoter.

It may also be advantageous for the promoters to be inducible so that the levels of expression of the heterologous gene can be regulated during the lifetime of the cell. Inducible means that the levels of expression obtained using the promoter can be regulated.

Any of the above promoters may be modified by the addition of further regulatory sequences, for example enhancer sequences. Chimeric promoters may also be used comprising sequence elements from two or more different promoters.

Alternatively (or in addition), the regulatory sequences may be cell specific such that the gene of interest is only expressed in cells of use in the present invention. Such cells include, for example, APCs and T-cells.

If required, a small aliquot of cells may be tested for up-regulation of Notch signalling activity as described above. The cells may be prepared for administration to a patient or incubated with T-cells in vitro (ex vivo).

Assays of Immune Response and Tolerisation

Any of the assays described above (see “Assays”) can be adapted to monitor or to detect reduced reactivity and tolerisation in immune cells, and to detect suppression and enhancement of immune responses for use in clinical applications.

Immune cell activity may be monitored by any suitable method known to those skilled in the art. For example, cytotoxic activity may be monitored. Natural killer (NK) cells will demonstrate enhanced cytotoxic activity after activation. Therefore any drop in or stabilisation of cytotoxicity will be an indication of reduced reactivity. Once activated, leukocytes express a variety of new cell surface antigens. NK cells, for example, will express transferrin receptor, HLA-DR and the CD25 IL-2 receptor after activation. Reduced reactivity may therefore be assayed by monitoring expression of these antigens.

Hara et al. Human T-cell Activation: III, Rapid Induction of a Phosphorylated 28 kD/32 kD Disulfide linked Early Activation Antigen (EA-1) by 12-0-tetradecanoyl Phorbol-13-Acetate, Mitogens and Antigens, J. Exp. Med., 164:1988 (1986), and Cosulich et al. Functional Characterization of an Antigen (MLR3) Involved in an Early Step of T-Cell Activation, PNAS, 84:4205 (1987), have described cell surface antigens that are expressed on T-cells shortly after activation. These antigens, EA-1 and MLR3 respectively, are glycoproteins having major components of 28 kD and 32 kD. EA-1 and MLR3 are not HLA class II antigens and an MLR3 Mab will block IL-1 binding. These antigens appear on activated T-cells within 18 hours and can therefore be used to monitor immune cell reactivity.

Additionally, leukocyte reactivity may be monitored as described in EP 0325489, which is incorporated herein by reference. Briefly this is accomplished using a monoclonal antibody (“Anti-Leu23”) which interacts with a cellular antigen recognised by the monoclonal antibody produced by the hybridoma designated as ATCC No. HB-9627.

Anti-Leu 23 recognises a cell surface antigen on activated and antigen stimulated leukocytes. On activated NK cells, the antigen, Leu 23, is expressed within 4 hours after activation and continues to be expressed as late as 72 hours after activation. Leu 23 is a disulfide-linked homodimer composed of 24 kD subunits with at least two N-linked carbohydrates.

Because the appearance of Leu 23 on NK cells correlates with the development of cytotoxicity and because the appearance of Leu 23 on certain T-cells correlates with stimulation of the T-cell antigen receptor complex, Anti-Leu 23 is useful in monitoring the reactivity of leukocytes.

Further details of techniques for the monitoring of immune cell reactivity may be found in: ‘The Natural Killer Cell’ Lewis C. E. and J. O'D. McGee 1992. Oxford University Press; Trinchieri G. ‘Biology of Natural Killer Cells' Adv. Immunol. 1989 vol 47 pp 187-376; ‘Cytokines of the Immune Response’ Chapter 7 in “Handbook of Immune Response Genes”. Mak T. W. and J. J. L. Simard 1998, which are incorporated herein by reference.

Preparation of Regulatory T cells (and B cells) ex vivo

The techniques described below are described in relation to T cells, but are equally applicable to B cells. The techniques employed are essentially identical to that described for APCs alone except that T cells are generally co-cultured with the APCs. However, it may be preferred to prepare primed APCs first and then incubate them with T cells. For example, once the primed APCs have been prepared, they may be pelleted and washed with PBS before being resuspended in fresh culture medium. This has the advantage that if, for example, it is desired to treat the T cells with a different substance(s), then the T cell will not be brought into contact with the different substance(s) used with the APC. Once primed APCs have been prepared, it is not always necessary to administer any substances to the T cell since the primed APC is itself capable of modulating immune responses or inducing immunotolerance leading to increased Notch or Notch ligand expression in the T cell, presumably via Notch/Notch ligand interactions between the primed APC and T cell.

Incubations will typically be for at least 1 hour, preferably at least 3, 6, 12, 24, 48 or 36 or more hours, in suitable culture medium at 37° C. The progress of Notch signalling may be determined for a small aliquot of cells using the methods described above. T cells transfected with a nucleic acid construct directing the expression of, for example Delta, may be used as a control. Modulation of immune responses/tolerance may be determined, for example, by subsequently challenging T cells with antigen and measuring IL-2 production compared with control cells not exposed to APCs.

Primed T cells or B cells may also be used to induce immunotolerance in other T cells or B cells in the absence of APCs using similar culture techniques and incubation times.

Alternatively, T cells may be cultured and primed in the absence of APCs by use of APC substitutes such as anti-TCR antibodies (e.g. anti-CD3) with or without antibodies to costimulatory molecules (e.g. anti-CD28) or alternatively T cells may be activated with MHC-peptide complexes (e.g. tetramers).

Induction of immunotolerance may be determined by subsequently challenging T cells with antigen and measuring IL-2 production compared with control cells not exposed to APCs.

T cells or B cells which have been primed in this way may be used according to the invention to promote or increase immunotolerance in other T cells or B cells.

Various preferred features and embodiments of the present invention will now be described in more detail by way of non-limiting examples.

### EXAMPLES

### Example 1

### Preparation of Modulator of Notch Signalling (hDelta1-IgG4Fc Fusion Protein)

A fusion protein comprising the extracellular domain of human Delta1 fused to the Fc domain of human IgG4 (“hDelta1-IgG4Fc”) was prepared by inserting a nucleotide sequence coding for the extracellular domain of human Delta1 (see, eg Genbank Accession No AF003522) into the expression vector pCONγ (Lonza Biologics, Slough, UK) and expressing the resulting construct in CHO cells.

i) Cloning

A 1622 bp extracellular (EC) fragment of human Delta-like ligand 1 (hECDLL-1; see GenBank Accession No AF003522) was gel purified using a Qiagen QIAquick™ Gel Extraction Kit (cat 28706) according to the manufacturer's instructions. The fragment was then ligated into a pCR Blunt cloning vector (Invitrogen, UK) cut HindIII-BsiWI, thus eliminating a HindIII, BsiWI and ApaI site.

The ligation was transformed into DH5α cells, streaked onto LB+Kanamycin (30 ug/ml) plates and incubated at 37° C. overnight. Colonies were picked from the plates into 3 ml LB+Kanamycin (30 ugml⁻¹) and grown up overnight at 37° C. Plasmid DNA was purified from the cultures using a Qiagen Qiaquick Spin Miniprep kit (cat 27106) according to the manufacturer's instructions, then diagnostically digested with HindIII. A clone was chosen and streaked onto an LB+Kanamycin (30 ug/ml) plate with the glycerol stock of modified pCRBlunt-hECDLL-1 and incubated at 37° C. overnight. A colony was picked off this plate into 60 ml LB+Kanamycin (30 ug/ml) and incubated at 37° C. overnight. The culture was maxiprepped using a Clontech Nucleobond Maxi Kit (cat K3003-2) according to the manufacturer's instructions, and the final DNA pellet was resuspended in 300 ul dH₂O and stored at −20° C.

5 ug of modified pCR Blunt-hECDLL-1 vector was linearised with HindIII and partially digested with ApaI. The 1622 bp hECDLL-1 fragment was then gel purified using a Clontech Nucleospin® Extraction Kit (K3051-1) according to the manufacturer's instructions. The DNA was then passed through another Clontech Nucleospin® column and followed the isolation from PCR protocol, concentration of sample was then checked by agarose gel analysis ready for ligation.

Plasmid pconγ (Lonza Biologics, UK) was cut with HindIII-ApaI and the following oligos were ligated in:
agcttgcggc cgcgggccca gcggtggtgg acctcactga gaagctagag gcttccacca aaggcc acgccg gcgcccgggt cgccaccacc tggagtgact cttcgatctc cgaaggtggt tt
   (SEQ ID NOS: 4 and 5)

The ligation was transformed into DH5α cells and LB+Amp (100 ug/ml) plates were streaked with 200 ul of the transformation and incubated at 37° C. overnight. The following day 12 clones were picked into 2×YT+Ampicillin (100 ugml⁻¹) and grown up at 37° C. throughout the day. Plasmid DNA was purified from the cultures using a Qiagen Qiaquick Spin Miniprep kit (cat 27106) and diagnostically digested with NotI. A clone (designated “pDev41”) was chosen and an LB+Amp (100 ug/ml) plate was streaked with the glycerol stock of pDev41 and incubated at 37° C. overnight. The following day a clone was picked from this plate into 60 ml LB+Amp (100 ug/ml) and incubated with shaking at 37° C. overnight. The clone was maxiprepped using a Clontech Nucleobond Maxi Kit (cat K3003-2) according to the manufacturer's instructions and stored at −20° C. The pDev41 clone 5 maxiprep was then digested with ApaI-EcoRI to generate the IgG4Fc fragment (1624 bp). The digest was purified on a 1% agarose gel and the main band was cut out and purified using a Clontech Nucleospin Extraction Kit (K3051-1).

The polynucleotide was then cloned into the polylinker region of pEE14.4 (Lonza Biologics, UK). 5 ug of the maxiprep of pEE14.4 was digested with HindIII-EcoRI, and the product was gel extracted and treated with alkaline phosphatase.

ii) Generation of Expression Constructs

A 3 fragment ligation was set up with pEE14.4 cut HindIII-EcoRI, ECDLL-1 from modified pCR Blunt (HindIII-ApaI) and the IgG4Fc fragment cut from pDev4l (ApaI-EcoRI). This was transformed into DH5α cells and LB+Amp (100 ug/ml) plates were streaked with 200 ul of the transformation and incubated at 37 C overnight. The following day 12 clones were picked into 2×YT+Amp (100 ug/ml) and minipreps were grown up at 37° C. throughout the day. Plasmid DNA was purified from the preps using a Qiagen Qiaquick spin miniprep kit (Cat No 27106), diagnostically digested (with EcoRI and HindIII) and a clone (clone 8; designated “pDev44”) was chosen for maxiprepping. The glycerol stock of pDev44 clone 8 was streaked onto an LB+Amp (100 ugml⁻¹) plate and incubated at 37° C. overnight. The following day a colony was picked into 60 ml LB+Amp (100 ugml⁻¹) broth and incubated at 37° C. overnight. The plasmid DNA was isolated using a Clontech Nucleobond Maxiprep Kit (Cat K3003-2).

iii) Addition of Optimal KOZAK Sequence

A Kozak sequence was inserted into the expression construct as follows. Oligonucleotides were kinase treated and annealed to generate the following sequences:

(SEQ ID NOS: 6 and 7)

AGCTTGCCGCCACCATGGGCAGTCGGTGCGCGCTGGCCCTGGCGGTGCTC
ACGGCGGTGGTACCCGTCAGCCACGCGCGACCGGGACCGC

(SEQ ID NOS: 8 and 9)
TCGGCCTTGCTGTGTCAGGTCTGGAGCTCTGGGGTGTT
CACGAGAGCCGGAACGACACAGTCCAGACCTCGAGACCCCACAAGC

pDev44 was digested with HindIII-BstBI, gel purified and treated with alkaline phosphatase. The digest was ligated with the oligos, transformed into DH5α cells by heat shock. 200 ul of each transformation were streaked onto LB+Amp plates (100 ug/ml) and incubated at 37° C. overnight. Minipreps were grown up in 3 ml 2×YT+Ampicillin (100 ugml⁻¹). Plasmid DNA was purified from the minipreps using a Qiagen Qiaquick spin miniprep kit (Cat No 27106) and diagnostically digested with NcoI. A clone (pDev46) was selected and the sequence was confirmed. The glycerol stock was streaked, broth grown up and the plasmid maxiprepped.

iv) Transfection

Approx 100 ug pDev46 Clone 1 DNA was linearised with restriction enzyme Pvu I. The resulting DNA preparation was cleaned up using phenol/chloroform/IAA extraction followed by ethanol wash and precipitation. The pellets were resuspended in sterile water and linearisation and quantification was checked by agarose gel electrophoresis and UV spectrophotometry.

40 ug linearised DNA (pDev46 Clone 1) and 1×10⁷ CHO-K1 cells were mixed in serum free DMEM in a 4 mm cuvette, at room temp. The cells were then electroporated at 975 uF 280 volts, washed out into non-selective DMEM, diluted into 96 well plates and incubated. After 24 hours media were removed and replaced with selective media (25 uM L-MSX). After 6 weeks media were removed and analysed by IgG4 sandwich ELISA. Selective media were replaced. Positive clones were identified and passaged in selective media 25 um L-MSX.

v) Expression

Cells were grown in selective DMEM (25 um L-MSX) until semi-confluent. The media was then replaced with serum free media (UltraCHO) for 3-5 days. Protein (hDelta1-IgG4Fc fusion protein) was purified from the resulting media by FPLC (Protein A column).

The amino acid sequence of the resulting expressed fusion protein was as follows (SEQ ID NO:10):

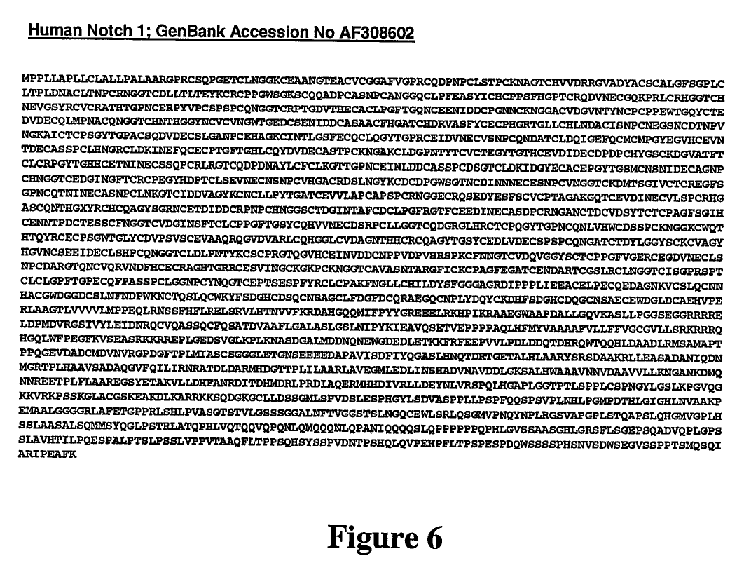

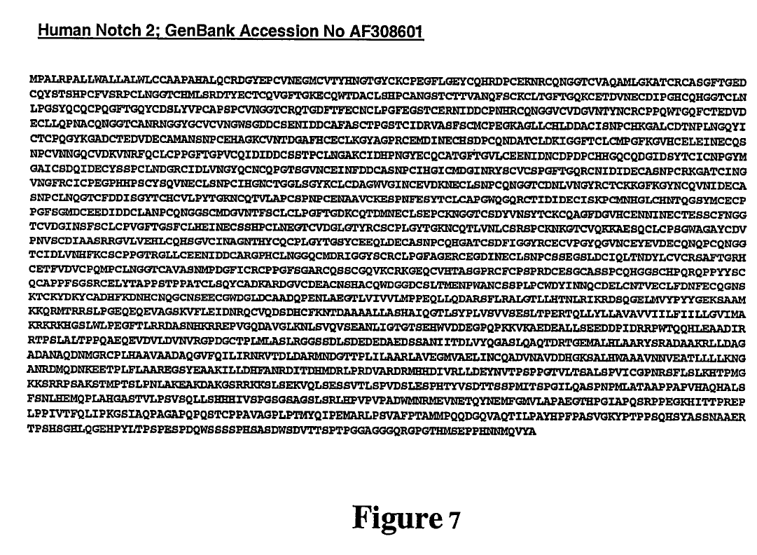

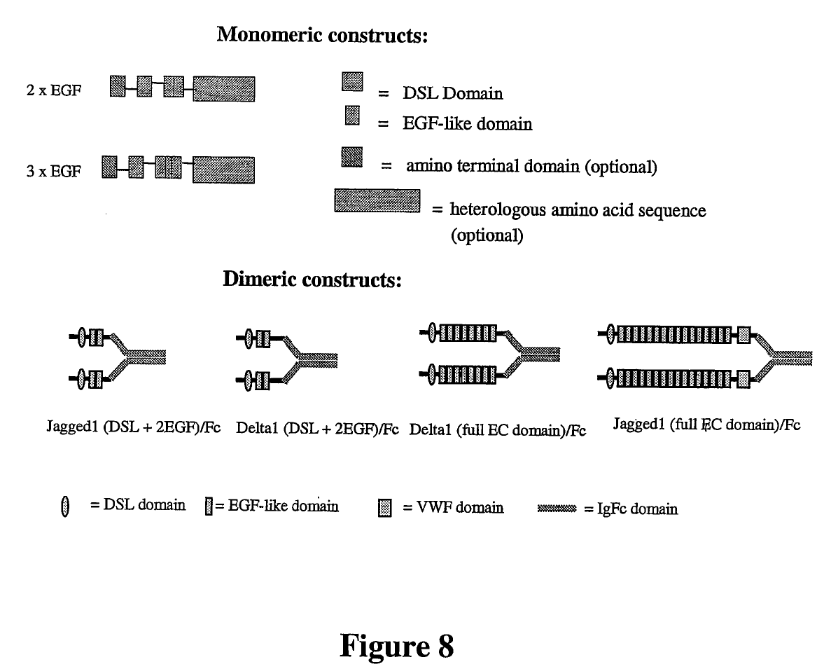

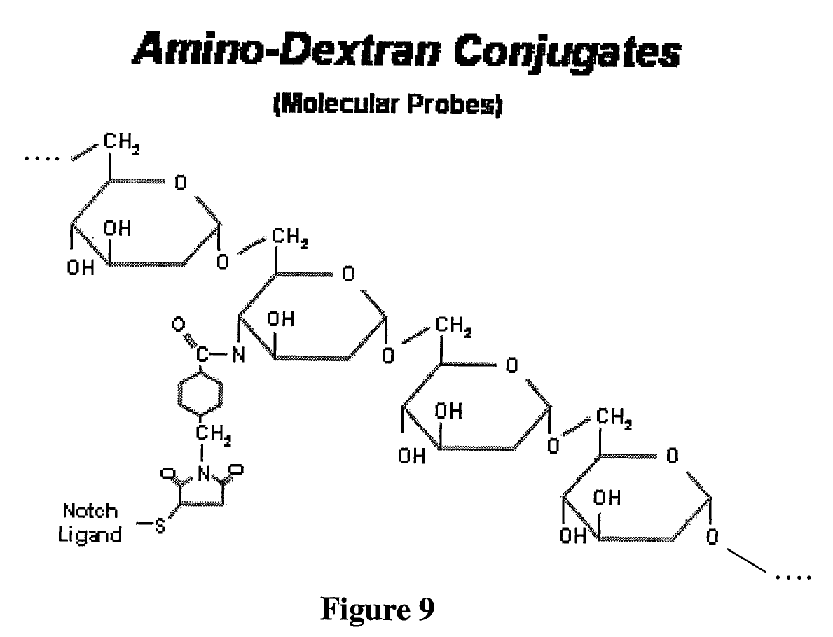

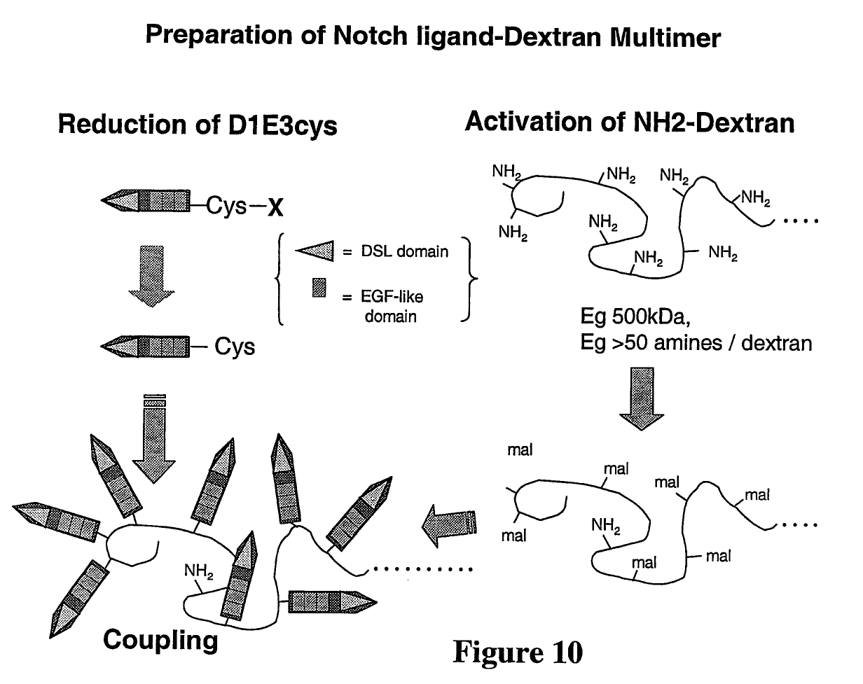

MGSRCALALAVLSALLCQVWSSGVFELKLQEFVNKKGLLGNRNCCRGGAG
PPPCACRTFFRVCLKHYQASVSPRPPCTYGSAVTPVLGVDSFSLPDGGGA
DSAFSNPIRFPFGFTWPGTFSLIIEALHTDSPDDLATENPERLISRLATQ
RHLTVGEEWSQDLHSSGRTDLKYSYRFVCDEHYYGEGCSVFCRPRDDAFG
HFTCGERGEKVCNPGWKGPYCTEPICLPGCDEQHGFCDKPGECKCRVGWQ
GRYCDECIRYPGCLHGTCQQPWQCNCQEGGWGGLFCNQDLNYCTHHKPCK
NGATCTNTGQGSYTCSCRPGYTGATCELGIDECDPSPCKNGGSCTDLENS
YSCTCPPGFYGKICELSAMTCADGPCFNGGRCSDSPDGGYSCRCPVYGSG
FNCEKKIDYCSSSPCSNGAKCVDLGDAYLCRCQAGFSGRHCDDNVDDCAS
SPCANGGTCRDGVNDFSCTCPPGYTGRNCSAPVSRCEHAPCHNGATCHER
GHGYVCECARGYGGPNCQFLLPELPPGPAVVDLTEKLEASTKGPSVFPLA
PCSRSTSESTAALGCLVKDYFPEPVTVSWNSGALTSGVHTFPAVLQSSGL
YSLSSVVTVPSSSLGTKTYTCNVDHKPSNTKVDKRVESKYGPPCPSCPAP
EFLGGPSVFLFPPKPKDTLMISRTPEVTCVVVDVSQEDPEVQFNWYVDGV
EVHNAKTKPREEQFNSTYRVVSVLTVLHQDWLNGKEYKCKVSNKGLPSSI
EKTISKAKGQPRPEPQVYTLPPSQEEMTKNQVSLTCLVKGFYPSDIAVEW
ESNGQPENNYKTTPPVLDSDGSFFLYSRLTVDKSRWQEGNVFSCSVMHEA
LHNHYTQKLSLSLSGK

Wherein the first underlined sequence is the signal peptide (cleaved from the mature protein) and the second underlined sequence is the IgG4 Fc sequence. The protein normally exists as a dimer linked by cysteine disulphide bonds (see eg schematic representations in FIG. 6).

The fusion protein is linked to polymer elements such as PEG as described above to provide the final conjugate.

### Example 2

### Preparation of Modulator of Notch Signalling: Truncated Human Jagged1 Fusion Protein (hJagged1EGF1&2-IgG4Fc)

As described in WO A fusion protein capable of acting as an inhibitor of Notch signalling comprising human jagged1 sequence up to the end of EGF2 (leader sequence, amino terminal, DSL, EGF1+2) fused to the Fc domain of human IgG4 (“hJagged1(EGF1+2)-IgG4Fc”) was prepared by inserting a nucleotide sequence coding for human Jagged1 from ATG through to the end of the second EGF repeat (EGF2) into the expression vector pCONγ (Lonza Biologics, Slough, UK) to add the IgG4 Fc tag. The full fusion protein was then shuttled into the Glutamine Synthetase (GS) selection system vector pEE14.4 (Lonza Biologics). The resulting construct was transfected and expressed in CHO-K1 cells (Lonza Biologics).

1. Cloning

i) Preparation of DNA-pDEV 47 and pDEV20

Human Jagged1 was cloned into pcDNA3.1 (Invitrogen) to give plasmid pLOR47.

The Jagged 1 sequence from pLOR47 was aligned against full length human jagged1 (GenBank U61276) and found to have only a small number of apparently silent changes.

Plasmid pLOR47 was then modified to remove one of two DraIII sites (whilst maintaining and replacing the amino acid sequence for full extracellular hjagged1) and add a BsiWI site after for ease of subsequent cloning. The resulting plasmid was named pDEV20.

Plasmid pLOR47 was cut with DraIII. This removed a 1.7 kb fragment comprising the 3′ end of the extracellular, the transmembrane and intracellular regions of hjagged1 as well as part of the vector sequence leaving a larger fragment of 7.3 kbp of the main vector backbone with almost all of the extracellular region (EC) of hjagged1. The cut DNA was run out on an agarose gel, the larger fragment excised and gel purified using a Qiagen QIAquick™ Gel Extraction Kit (cat 28706) according to the manufacturer's instructions.

A pair of oligonucleotides were ordered such that when ligated together gave a double stranded piece of DNA that had a compatible sticky end for DraIII at the 5′ end and recreated the original restriction site. This sequence was followed by a BsiWI site then another compatible sticky end for DraIII at the 3′ end that did not recreate the restriction site.

(SEQ ID NOS: 11 and 12)

```
i.e. DraIII BsiWI DraIII
              gtg ctg tta ccc gta cgg ta
      gaa cac gac aat ggg cat gc
```

This oligo pair was then ligated into the DraII cut pLOR47 thus maintaining the 5′ DraIII site, inserting a BsiWI and eliminating the 3′DraIII site. The resulting plasmid was named pDEV20.

ii) Preparing hjagged1 IgG4 FC Fusion DNA:

A three fragment ligation was necessary to reassemble full hjagged1 EC sequence with addition of a modified 5′ Kozak sequence and 5′ end repair together with repair of 3′end.

Fragment 1: EC hjagged Sequence:

pDev 20 was cut RsrII-DraIII giving rise to 3 fragments; 1270+2459+3621 bp. The fragments were run out on an agarose gel, the 2459 bp band excised and the DNA gel purified using a Qiagen QIAquick™ Gel Extraction Kit (cat 28706) according to the manufacturer's instructions. This contained hjagged1 sequence—with loss of 3′ sequence (up to the RsrII site) and loss of some 5′sequence at the end of the EC region.

Fragment 2: Modified Kozak Sequence:

pUC 19 (Invitrogen) was modified to insert new restriction enzyme sites and also introduce a modified Kozak with 5′ hjagged1 sequence. The new plasmid was named pLOR49. pLOR49 was created by cutting pUC19 vector HindIII EcoRI and ligating in 4 oligonucleotides (2 oligo pairs). One pair has a HindIII cohesive end followed by an optimal Kozac and 5′hJagged 1 sequence followed by RsrII cohesive end.

```
(SEQ ID NOS: 13 and 14)
i.e. HindIII optimal Kozak + 5′hJagged1 sequence RsrII
              ag ctt gcc gcc acc atg ggt tcc cca cgg aca cgc ggc cg
                        a cgg cgg tgg tac cca agg ggt gcc tgt gcg ccg gcc ag
```

The other pair has a cohesive RsrII end then DraIII, KpnI, BsiWI sites followed by a cohesive EcoRI site.

(SEQ ID NOS: 15 and 16)

```
i.e. RsrII DraII KpnI BsiWI EcoRI
          gtc cgc acc ttg tgg gta ccc gta cgg
                  gcg tgg aac acc cat ggg cat gcc tta a
```

pLOR49 thus is a pUC19 back bone with the HindIII site followed by optimal Kozac and 5′hJagged1 sequence and introduced unique RsrII, Dra III, KpnI, BsiWI sites before recreating the EcorI site.

Plasmid pLOR49 was then cut RsrII-BsiWI to give a 2.7 kbp vector backbone fragment that was run out on an agarose gel, the band excised and the DNA gel purified using a Qiagen QIAquick™ Gel Extraction Kit (cat 28706) according to the manufacturer's instructions.

Fragment 3: Generation of 3′ hjagged1 EC with BsiWI Site PCR Fragment:

pLOR47 was used as a template for PCR to amplify up hJagged1 EC and add a 3′ BsiWI site.

5′ primer from RsrII site of hJagged I

3′ site up to end of hJagged1 EC with BsiWI site stitched on 3′

The resulting fragment was cut with DraIII and BsiWI to give a fragment around 600 bp. This was run out on an agarose gel, the band excised and the DNA gel purified using a Qiagen QIAquick™ Gel Extraction Kit (cat 28706) according to the manufacturer's instructions.

The three fragments described above;
1) 2459 bp h Jagged1 fragment from pDev 20 cut RsrII-DraIII
2) 2.7 kbp optimised Kozak and 5′ hjagged1 from Lor 49 cut RsrII-BsiWI
3) 600 bp 3′EC hjagged1 PCR fragment cut DraIII-BsiWI
   were then ligated together to give plasmid pDEV21.
   iii) Further Ligation (PDEV10):

To exclude any extraneous sequences a further 3 fragment ligation was carried out to drop straight into the vector pCONγ 4 (Lonza Biologics, Slough, UK).

Fragment 1: Plasmid pDEV21-4 was cut HindIII-BglII to give 4958 bp+899 bp fragments. These were run out on an agarose gel, the smaller 889 bp fragment band was excised and the DNA gel purified using a Qiagen QIAquick™ Gel Extraction Kit (cat 28706) according to the manufacturer's instructions.

Fragment 2: pCONγ 4 (Lonza Biologics) was cut Hind III-ApaI to give a 6602 bp vector fragment—missing the first 5 amino acids of IgG4 FC. The fragment band was excised and the DNA gel purified using a Qiagen QIAquick™ Gel Extraction Kit (cat 28706) according to the manufacturer's instructions.

Fragment 3: A linker oligonucleotide pair was ordered to give a tight junction between the end of hJagged1 EGF2 and the 3′ start of IgG4 FC, with no extra amino acids introduced.

| | | |
|---|---|---|
| i.e. | BglII D L A S T K G ApaI | DL = |
| | gat ctc gct tcc acc aag ggc c | hJagged1 |
| | ag cga agg tgg ttc | sequence |
| | | remain- |
| | | der = |
| | | IgG4 FC |
| | | sequence |

(SEQ ID NOS: 17 and 18)

The three fragments described above;
1. 899 bp hjagged1 fragment pDEV21-4 cut HindIII-BglII
2. 6602 bp pConGamma vector backbone cut HindIII ApaI
3. oligo linker BglII-ApaI
   were ligated together to give plasmid pDEV10.

Ligated DNA was transformed into competent DH5alpha (Invitrogen), plated onto LB amp paltes and incubated at 37 degres overnight. A good ratio was evident between control and vector plus insert pates therefore only 8 colonies were picked into 10 ml LB amp broth and incubated at 37 overnight. Glycerol broths were made and the bacterial pellets were frozen at −20degrees. Later plasmid DNA was extracted using Qiagen miniprep spin kit and were diagnostically digested with ScaI. Clones 2,4, and 5 looked correct so clone 2 was steaked onto LB Amp plates and inoculate 1/100 into 120 ml LB+amp broth. Plates and broths were incubated at 37 degrees overnight. Glycerol broths were made from the broths and pellets frozen to maxiprep later. Plasmid DNA was extracted Clontech Maxiprep, diagnostic digests were set up with ScaI and the DNA was diluted for quantification and quality check by UV spectrophotometry.

iv) pDev11 Cloning:

The coding sequence for hjagged1 EGF1+2 IgG4 FC fusion was shuttled out of pCONγ 4 (Lonza Biologics) into pEE 14.4 (Lonza Biologics) downstream of the hCMV promoter region (hCMV-MIE) and upstream of SV40 polyadenylation signal, to enable stable cell lines to be selected using the GS system (Lonza Biologics).

v) Insert:

pDEV10 clone 2 was cut HindIII-EcoRI giving rise to 2 fragment s 5026 bp+2497 bp. The 2497 bp contained the coding sequence for hjagged1 EGF1+2 IgG4 FC fusion and so was excised from an agarose gel and the DNA gel purified using a Qiagen QIAquick™ Gel Extraction Kit (cat 28706) according to the manufacturer's instructions.

vi) Vector:

pEE14.4 (Lonza Biologics) was cut HindIII-EcoRI to remove the IgG4 FC sequence giving 2 fragments 5026 bp+1593 bp. The larger 5026 bp fragment was excised from an agarose gel and the DNA gel purified using a Qiagen QIAquick™ Gel Extraction Kit (cat 28706) according to the manufacturer's instructions. The pEE14.4 vector backbone and the hjagged1 EGF1+2 IgG4 FC fusion insert were ligated to give the final transfection plasmid pDEV11.

The ligation was transformed into DH5α cells, streaked onto LB+Ampicillin (100 ug/ml) plates and incubated at 37° C. overnight. Colonies were picked from the plates into 7 ml LB+Ampicillin (100 ug/ml) and grown up shaking overnight at 37° C. Glycerol broths were made and the plasmid DNA was purified from the cultures using a Qiagen Qiaquick Spin Miniprep kit (cat 27106) according to the manufacturer's instructions. The DNA was then diagnostically digested with SapI.

vii) Maxiprep for Transfection:

A correct clone (clone 1) was chosen and 100 ul of the glycerol stock was inoculated into 100 ml LB+Ampicillin (100 ug/ml), and also streaked out onto LB+Ampicillin (100 ug/ml) plates. Both plate and broth were incubated at 37° C. overnight.

The plates showed pure growth; therefore the culture was maxi-prepped using a Clontech Nucleobond Maxi Kit (cat K3003-2) according to the manufacturer's instructions. The final DNA pellet was resuspended in 500 ul dH₂O.

A sample of pLOR 11 clone1 DNA was then diluted and the concentration and quality of DNA assessed by UV spectrophotometry. A sample was also diagnostically digested with SapI, and gave bands of the correct size.

viii) Linearisation of DNA:

Approx 100 ug pDev11 Clone 1 DNA was linearised with restriction enzyme Pvu I. The resulting DNA preparation was cleaned up using phenol/chloroform/IAA extraction followed by ethanol wash and precipitation inside a laminar flow hood. The pellets were resuspended in sterile water. Linearisation was checked by agarose gel electrophoresis while quantification and quality were assessed by UV spectrophotometry at 260 and 280 nm.

2. Transfection

40 ug linearised DNA (pDev11 Clone 1) and 1×10⁷ CHO-K1 cells (Lonza) were mixed in 500 ul of serum free DMEM in a 4 mm cuvette, at room temp. The cells were then electroporated at 975 uF 280 volts, washed out into 60 ml of non-selective DMEM (DMEM/glut/10% FCS). From this dilution 6×96 well pates were inoculated with 50 ul per well. A 1/4 dilution of the original stock was made and from this 8×96 well pates were inoculated with 50 ul per well. A further 1/10 dilution was made from the second stock, and from this 12×96 well pates were inoculated with 50 ul per well. Plates were incubated at 37 degrees C 5% CO2 overnight. After 24 hours the media was removed and replaced with 200 ul of selective media (25 uM L-MSX).

Between 4-6 weeks post transfection media was removed from the plates for analysis by IgG4 sandwich ELISA. Selective media were replaced. Positive clones were identified, passaged and expanded in selective media 25 um L-MSX.

3. Expression

Cells were grown in selective DMEM (25 um L-MSX) until semi-confluent. The media was then replaced with serum free media (UltraCHO; BioWhittaker) for 3-5 days. Protein (hJagged1EGF1+2-IgG4Fc fusion protein) was purified from the resulting media by FPLC.

Amino Acid Sequence of the Expressed Fusion Protein (hjagged1 EGF1+2 IgG4 FC):

(SEQ ID NO: 19)

| | |
|---|---|
| 1 | mrsprtrgrs grplslllal lcalrakvcg asgqfeleil |
| | smqnvngelq ngnccggarn |
| 61 | pgdrkctrde cdtyfkvclk eyqsrvtagg pcsfgsgstp |
| | viggntfnlk asrgndpnri |
| 121 | vlpfsfawpr sytllveawd ssndtvqpds iiekashsgm |
| | inpsrqwqtl kqntgvahfe |
| 181 | yqirvtcddy yygfgcnkfc rprddffghy acdqngnktc |
| | megwmgpecn raicrqgcsp |
| 241 | khgscklpgd crcqygwqgl ycdkciphpg cvhgicnepw |
| | qclcetnwgg qlcdkdlvra |
| 301 | stkgpsvfpl apcsrstses taalgclvkd yfpepvtvsw |
| | nsgaltsgvh tfpavlgssg |
| 361 | lyslssvvtv pssslgtkty tcnvdhkpsn tkvdkrvesk |
| | ygppcpscpa peflggpsvf |
| 421 | lfppkpkdtl misrtpevtc vvvdvsgedp evgfnwyvdg |
| | vevhnaktkp reegfnstyr |
| 481 | vvsvltvlhg dwlngkeykc kvsnkglpss iektiskakg |
| | qprepqvytl ppsgeemtkn |
| 541 | qvsltclvkg fypsdiavew esngqpenny kttppvldsd |
| | gsfflysrlt vdksrwgegn |
| 601 | vfscsvmhea lhnhytgksl slslgk |

Bold=hJagged1 extracellular domain leader sequence, amino terminal region, DSL and EGF 1+2, Underlined=IgG4 Fc sequence.

The protein is believed to exist as a dimer linked by cysteine disulphide bonds, with cleavage of the signal peptide.

The fusion protein is linked to polymer elements such as dextran or PEG as described above to provide the final conjugate.

### Example 3

A series of truncations of sequences for modulating Notch signalling, based on human Delta1 comprising varying numbers of EGF repeats, was prepared as follows:

A) Delta 1 DSL Domain Plus EGF Repeats 1-2

A human Delta 1 (DLL-1) deletion coding for the DSL domain and the first two only of the naturally occurring EGF repeats (ie omitting EGF repeats 3 to 8 inclusive) was generated by PCR from a DLL-1 extracellular (EC) domain/V5His clone (for the sequence of the human DLL-1 EC domain see Figures and, for example, Genbank Accession No. AF003522) using a primer pair as follows:

| | |
|---|---|
| DLac13: | |
| CACCAT GGGCAG TCGGTG CGCGCT GG; | (SEQ ID NO: 20) |
| and | |
| DLL1d3-8: | |
| GTAGTT CAGGTC CTGGTT GCAG | (SEQ ID NO: 21) |

PCR conditions were:

1 cycle at 95° C./3 minutes;

18 cycles of (95° C./1 minute, 60° C./1 minute, 72° C./2 minutes); and

1 cycle at 72° C./2 minutes.

The DNA was then isolated from a 1% agarose gel in 1× UN-Safe TAE (Tris/acetate/EDTA) buffer (MWG-Biotech, Ebersberg, Germany) and used as a template for

PCR with the following primers:

| | | |
|---|---|---|
| FcDL.4: | CACCAT GGGCAG TCGGTG | (SEQ ID NO: 22) |
| | CGCGCT GG; | |
| and | | |
| FcDLLd3-8: | GGATAT GGGCCC TTGGTG | (SEQ ID NO: 23) |
| | GAAGCG TAGTTC AGGTCC | |
| | TGGTTG CAG | |

PCR conditions were:

1 cycle at 94° C./3 minutes;

18 cycles of (94° C./1 minute, 68° C./1 minute, 72° C./2 minutes); and

1 cycle at 72° C./10 minutes.

The fragment was ligated into pCRbluntII.TOPO (Invitrogen) and cloned in TOP10 cells (Invitrogen). Plasmid DNA was generated using a Qiagen Minprep kit (QIAprep™) according to the manufacturer's instructions and the identity of the PCR products was confirmed by sequencing.

An IgFc fusion vector pCONγ (Lonza Biologics, UK) was cut with ApaI and HindIII then treated with shrimp alkaline phosphatase (Roche) and gel purified.

The DLL-1 deletions cloned in pCRbluntII were cut with HindIII (and EcoRV to aid later selection of the desired DNA product) followed by ApaI partial restriction. The sequences were then gel purified and ligated into the pCONγ vector which was cloned into TOP10 cells.

Plasmid DNA was generated using a Qiagen Minprep kit (QIAprep™) according to the manufacturer's instructions.

The resulting construct (pCONγ HDLL1 EGF1-2) coded for the following DLL-1 amino acid sequence fused to the IgG Fc domain encoded by the pCONγ vector.

| | |
|---|---|
| MGSRCALALAVLSALLCQVWSSGVFELKLQEFVNKKGLLGNRNCCRGGAGPPPCACR | (SEQ ID NO: 24) |
| TFFRVCLKHYQASVSPEPPCTYGSAVTPVLGVDSFSLPDGGGADSAFSNPIRFPFGF | |
| TWPGTFSLIIEALHTDSPDDLATENPERLISRLATQRHLTVGEEWSQDLHSSGRTDL | |
| KYSYRFVCDEHYYGEGCSVFCRPRDDAFGHFTCGERGEKVCNPGWKGPYCTEPICLP | |
| GCDEQHGFCDKPGECKCRVGWQGRYCDECIRYPGCLHGTCQQPWQCNCQEGWGGLFC | |
| NQDLNY | |

(wherein the emboldened portion of the sequence which is single underlined is the DSL domain and the emboldened portions of the sequence which are double underlined are EGF repeats 1 and 2 respectively).
B) Delta 1 DSL Domain Plus EGF Repeats 1-3

A human Delta 1 (DLL-1) deletion coding for the DSL domain and the first three only of the naturally occurring EGF repeats (ie omitting EGF repeats 4 to 8 inclusive) was generated by PCR from a DLL-1 DSL plus EGF repeats 1-4 clone using a primer pair as follows:

| | |
|---|---|
| DLac13: | |
| CACCATGGGCAGTCGGTGCGCGCTGG; | (SEQ ID NO: 25) |
| and | |
| FcDLLd4-8: | |
| GGA TAT GGG CCC TTG GTG GAA GCC | (SEQ ID NO: 26) |
| TCG TCA ATC CCC AGC TCG CAG | |

PCR conditions were:

1 cycle at 94° C./3 minutes;

18 cycles of(94° C./1 minute, 68° C./1 minute, 72° C./2.5 minutes); and

1 cycle at 72° C./10 minutes

The DNA was then isolated from a 1% agarose gel in 1× U/V-Safe TAE (Tris/acetate/EDTA) buffer (MWG-Biotech, Ebersberg, Germany) and ligated into pCRbluntII.TOPO and cloned in TOP10 cells (Invitrogen). Plasmid DNA was generated using a Qiagen Minprep kit (QIAprep™) according to the manufacturer's instructions and the identity of the PCR products was confirmed by sequencing.

An IgFc fusion vector pCONγ (Lonza Biologics, UK) was cut with ApaI and HindIII then treated with shrimp alkaline phosphatase (Roche) and gel purified.

The DLL-1 deletions cloned in pCRbluntII were cut with HindIII followed by ApaI partial restriction. The sequences were then gel purified and ligated into the pCONγ vector which was cloned into TOP10 cells.

Plasmid DNA was generated using a Qiagen Minprep kit (QIAprep™) according to the manufacturer's instructions and the identity of the PCR products was confirmed by sequencing.

The resulting construct (pCONγ hDLL1 EGF1-3) coded for the following DLL-1 sequence fused to the IgG Fc domain coded by the pCONγ vector.

| | |
|---|---|
| MGSRCALALAVLSALLCQVWSSGVFELKLQEFVNKKGLLGNRNCCRGGAGPPPCACR | (SEQ ID NO: 27) |
| TFFRVCLKHYQASVSPEPPCTYGSAVTPVLGVDSFSLPDGGGADSAFSNPIRFPFGF | |
| TWPGTFSLIIEALHTDSPDDLATENPERLISRLATQRHLTVGEEWSQDLHSSGRTDL | |
| KYSYRFVCDEHYYGEGCSVFCRPRDDAFGHFTCGERGEKVCNPGWKGPYCTEPICLP | |
| GCDEQHGFCDKPGECKCRVGWQGRYCDECIRYPGCLHGTCQQPWQCNCQEGWGGLFC | |
| NQDLNYCTHHKPCKNGATCTNTGQGSYTCSCRPGYTGATCELGIDE | |

(wherein the emboldened portion of the sequence which is single underlined is the DSL domain and the emboldened portions of the sequence which are double underlined are EGF repeats 1 to 3 respectively).
C) Delta 1 DSL Domain Plus EGF Repeats 1-4

A human Delta 1 (DLL-1) deletion coding for the DSL domain and the first four only of the naturally occurring EGF repeats (ie omitting EGF repeats 5 to 8 inclusive) was generated by PCR from a DLL-1 EC domain/V5His clone using a primer pair as follows:

| | |
|---|---|
| DLac13: | |
| CACCAT GGGCAG TCGGTG CGCGCT GG | (SEQ ID NO: 28) |
| and | |
| DLL1d5-8: | |
| GGTCAT GGCACT CAATTC ACAG | (SEQ ID NO: 29) |

1 cycle at 95° C./3 minutes;

18 cycles of(95° C./1 minute, 60° C./1 minute, 72° C./2.5 minutes); and

1 cycle at 72° C./10 minutes.

The DNA was then isolated from a 1% agarose gel in 1× U/V-Safe TAE (Tris/acetate/EDTA) buffer (MWG-Biotech, Ebersberg, Germany) and used as a template for PCR using the following primers:

| | | |
|---|---|---|
| FcDL.4: | CACCAT GGGCAG TCGGTG | (SEQ ID NO: 30) |
| | CGCGCT GG; | |
| and | | |
| FcDLLd5-8: | GGATAT GGGCCC TTGGTG | (SEQ ID NO: 31) |
| | GAAGCG GTCATG GCACTC | |
| | AATTCA CAG | |

PCR conditions were:

1 cycle at 94° C./3 minutes;

18 cycles of(94° C./1 minute, 68° C./1 minute, 72° C./2.5 minutes); and

1 cycle at 72° C./10 minutes.

The fragment was ligated into pCRbluntII.TOPO and cloned in TOP10 cells (Invitrogen). Plasmid DNA was generated using a Qiagen Minprep kit (QIAprep™) according to the manufacturer's instructions and the identity of the PCR products was confirmed by sequencing.

An IgFc fusion vector pCONγ (Lonza Biologics, UK) was cut with ApaI and HindIII then treated with shrimp alkaline phosphatase (Roche) and gel purified.

The DLL-1 deletions cloned in pCRbluntII were cut with HindIII (and EcoRV to aid later selection of the desired DNA product) followed by ApaI partial restriction. The sequences were then gel purified and ligated into the pCONγ vector which was cloned into TOP10 cells.

Plasmid DNA was generated using a Qiagen Minprep kit (QLAprep™) according to the manufacturer's instructions and the identity of the PCR products was confirmed by sequencing.

The resulting construct (pCONγ hDLL1 EGF1-4) coded for the following DLL-1 sequence fused to the IgG Fc domain coded by the pCONγ vector.

| | |
|---|---|
| MGSRCALALAVLSALLCQVWSSGVFELKLQEFVNKKGLLGNRNCCRGGAGPPPCACR | (SEQ ID NO: 32) |
| TFFRVCLKHYQASVSPEPPCTYGSAVTPVLGVDSFSLPDGGGADSAFSNPIRFPFGF | |
| TWPGTFSLIIEALHTDSPDDLATENPERLISRLATQRHLTVGEE+E,us WSQDLHSSGRTDL | |
| KYSYRFVCDEHYYGEGCSVFCRPRDDAFGHFTCGERGEKVCNPGWKGPYCTEPICLP | |
| GCDEQHGFCDKPGECKCRVGWQGRYC+E DECIRYPGCLHGTCQQPWQCNCQEGWGGLFC | |
| NQDLNYCTHHKPCKNGATCTNTGQGSYTCSCRPGYTGATCELGIDECDPSPCKNGGS | |
| CTDLENSYSCTCPPGFYGKICELSAMT | |

(wherein the emboldened portion of the sequence which is single underlined is the DSL domain and the emboldened portions of the sequence which are double underlined are EGF repeats 1 to 4 respectively).
D) Delta 1 DSL Domain Plus EGF Repeats 1-7

A human Delta 1 (DLL-1) deletion coding for the DSL domain and the first seven of the naturally occurring EGF repeats (ie omitting EGF repeat 8) was generated by PCR from a DLL-1 EC domainV5His clone using a primer pair as follows:

| | |
|---|---|
| DLac13: | |
| CACCAT GGGCAG TCGGTG CGCGCT GG; | (SEQ ID NO: 33) |
| and | |
| DLL1d8: | |
| CCTGCT GACGGG GGCACT GCAGTT C | (SEQ ID NO: 34) |

PCR conditions were:

1 cycle at 95° C./3 minutes;

18 cycles of (95° C./1 minute, 68° C./1 minute, 72° C./3 minutes); and

1 cycle at 72° C./10 minutes.

The DNA was then isolated from a 1% agarose gel in 1× UN-Safe TAE (Tris/acetate/EDTA) buffer (MWG-Biotech, Ebersberg, Germany) and used as a template for PCR using the following primers:

| | | |
|---|---|---|
| FcDL.4: | CACCAT GGGCAG TCGGTG | (SEQ ID NO: 35) |
| | CGCGCT GG; | |
| and | | |
| FCDLLd8: | GGATAT GGGCCC TTGGTG | (SEQ ID NO: 36) |
| | GAAGCC CTGCTG ACGGGG | |
| | GCACTG CAGTTC | |

PCR conditions were:

1 cycle at 94° C./3 minutes;

18 cycles of (94° C./1 minute, 68° C./1 minute, 72° C./3minutes); and

1 cycle at 72° C./10 minutes.

The fragment was ligated into pCRbluntII.TOPO and cloned in TOP10 cells (Invitrogen). Plasmid DNA was generated using a Qiagen Minprep kit (QIAprep™) according to the manufacturer's instructions and the identity of the PCR products was confirmed by sequencing.

An IgFc fusion vector pCONγ (Lonza Biologics, UK) was cut with ApaI and HindIII then treated with shrimp alkaline phosphatase (Roche) and gel purified.

The DLL-1 deletions cloned in pCRbluntII were cut with HindIII (and EcoRV to aid later selection of the desired DNA product) followed by ApaI partial restriction. The sequences were then gel purified and ligated into the pCONγ vector which was cloned into TOP10 cells.

Plasmid DNA was generated using a Qiagen Minprep kit (QIAprep™) according to the manufacturer's instructions and the PCR products were sequenced.

The resulting construct (pCONγ HDLL1 EGF1-7) coded for the following DLL-1 sequence fused to the IgG Fc domain coded by the pCONγ vector.

| | |
|---|---|
| MGSRCALALAVLSALLCQVWSSGVFELKLQEFVNKKGLLGNRNCCRGGAGPPPCACR | (SEQ ID NO: 37) |
| TFFRVCLKHYQASVSPEPPCTYGSAVTPVLGVDSFSLPDGGGADSAFSNPIRFPFGF | |
| TWPGTFSLIIEALHTDSPDDLATENPERLISRLATQRHLTVGEEWSQDLHSSGRTDL | |
| KYSYRFVCDEHYYGEGCSVFCRPRDDAFGHFTCGERGEKVCNPGWKGPYCTEPICLP | |
| GCDEQHGFCDKPGECKCRVGWQGRYCDECIRYPGCLHGTCQQPWQCNCQEGWGGLFC | |
| NQDLNYCTHHKPCKNGATCTNTGOGSYTCSCRPGYTGATCELGIDECDPSPCKNGGS | |
| CTDLENSYSCTCPPGFYGKICELSAMTCADGPCFNGGRCSDSPDGGYSCRCPVGYSG | |
| FNCEKKTDYCSSSPCSNGAKCVDLGDAYLCRCQAGFSGRHCDDNVDDCASSPCANGG | |
| TCRDGVNDFSCTCPPGYTGRNCSAPVSR | |

(wherein the emboldened portion of the sequence which is single underlined is the DSL domain and the emboldened portions of the sequence which are double underlined are EGF repeats 1 to 7 respectively).
E) Transfection and Expression
i) Transfection and Expression of Constructs of Constructs A, C and D

Cos 1 cells were separately transfected with each of the expression constructs from A, C and D above (viz pCONγ hDLL1 EGF1-2, pCONγ HDLL1 EGF1-4, pCONγ hDLL1 EGF1-7) and pCONγ control as follows:

In each case 3×10⁶ cells were plated in a 10 cm dish in Dulbecco's Modified Eagle's Medium (DMEM)+10% Fetal Calf Serum (FCS) and cells were left to adhere to the plate overnight. The cell monolayer was washed twice with 5 ml phosphate-buffered saline (PBS) and cells left in 8 ml OPTIMEM™ medium (Gibco/Invitrogen). 12 μg of the relevant construct DNA was diluted into 810 μl OPTIMEM medium and 14 μl Lipofectamine2000™ cationic lipid transfection reagent (Invitrogen) was diluted in 810 μl OPTIMEM medium. The DNA-containing and Lipofectamine2000 reagent-containing solutions were then mixed and incubated at room temperature for a minimum of 20 minutes, and then added to the cells ensuring an even distribution of the transfection mix within the dish. The cells were incubated with the transfection reagent for 6 hours before the media was removed and replaced with 20 ml DMEM+10% FCS. Supernatant containing secreted protein was collected from the cells after 5 days and dead cells suspended in the supernatant were removed by centrifugation (4,500 rpm for 5 minutes). The resulting expression products were designated: hDLL1 EGF1-2 Fc (from pCONγ hDLL1 EGF1-2), hDLL1 EGF1-4 Fc (from pCONγ HDLL1 EGF1-4) and hDLL1 EGF1-7 Fc (from pCONγ hDLL1 EGF1-7).

Expression of the Fc fusion proteins was assessed by western blot. The protein in 10 μl of supernatant was separated by 12% SDS-PAGE and blotted by semi dry apparatus on to Hybond™-ECL (Amersham Pharmacia Biotech) nitrocellulose membrane (17 V for 28 minutes). The presence of Fc fusion proteins was detected by Western blot using JDC14 anti-human IgG4 antibody diluted 1:500 in blocking solution (5% non-fat Milk solids in Tris-buffered saline with Tween 20 surfactant; TBS-T). The blot was incubated in this solution for 1 hour before being washed in TBS-T. After 3 washes of 5 minutes each, the presence of mouse anti-human IgG4 antibodies was detected using anti mouse IgG-HPRT conjugate antiserum diluted 1:10,000 in blocking solution. The blot was incubated in this solution for 1 hour before being washed in TBS-T (3 washes of 5 minutes each). The presence of Fc fusion proteins was then visualised using ECL™ detection reagent (Amersham Pharmacia Biotech).

The amount of protein present in 10 ml supernatant was assessed by comparing to Kappa chain standards containing 10 ng (7), 30 ng (8) and 100 ng (9) protein.

ii) Transfection and Expression of Constructs of Construct B

Cos 1 cells were transfected with the expression construct from B above (viz pCONγ HDLL1 EGF1-3) as follows:

7.1×10⁵ cells were plated in a T25 flask in Dulbecco's Modified Eagle's Medium (DMEM)+10% Fetal Calf Serum (FCS) and cells were left to adhere to the plate overnight. The cell monolayer was washed twice with 5 ml phosphate-buffered saline (PBS) and cells left in 1.14 ml OPTIMEM™ medium (Gibco/Invitrogen). 2.85 μg of the relevant construct DNA was diluted into 143 μl OPTIMEM medium and 14.3 μl Lipofectamine2000™ cationic lipid transfection reagent (Invitrogen) was diluted in 129 μl OPTIMEM medium and incubated at room temperature for 45 minutes. The DNA-containing and Lipofectamine2000 reagent-containing solutions were then mixed and incubated at room temperature for 15 minutes, and then added to the cells ensuring an even distribution of the transfection mix within the flask. The cells were incubated with the transfection reagent for 18 hours before the media was removed and replaced with 3 ml DMEM+10% FCS. Supernatant containing secreted protein was collected from the cells after 4 days and dead cells suspended in the supernatant were removed by centrifugation (1,200 rpm for 5 minutes). The resulting expression product was designated: hDLL1 EGF1-3 Fc (from pCONγ hDLL1 EGF1-3).

These fusion proteins are linked to polymers such as dextran or PEG as described above to provide the final conjugate.

### Example 4

i) Preparation of Modulator of Notch Signalling in form of Notch Ligand Extracellular Domain Fragment with Free Cysteine Tail for Polymer Coupling

A protein fragment comprising amino acids 1 to 332 (i.e. comprising DSL domain plus first 3 EGF repeats) of human Delta 1 (DLL-1; for sequence see GenBank Accession No AF003522) and ending with a free cysteine residue (“D1E3Cys”) was prepared as follows:

A template containing the entire coding sequence for the extracellular (EC) domain of human DLL-1 (with two silent mutations) was prepared by a PCR cloning strategy from a placental cDNA library made from placental polyA+RNA (Clontech; cat no 6518-1) and combined with a C-terminal V5HIS tag in a pCDNA3.1 plasmid (Invitrogen, UK) The template was cut HindIII to PmeI to provide a fragment coding for the EC domain and this was used as a template for PCR using primers as follows:

| | | |
|---|---|---|
| 5′-primer: | CAC CAT GGG CAG TCG | (SEQ ID NO: 38) |
| | GTG CGC GCT GG | |
| 3′-primer: | GTC TAC GTT TAA ACT | (SEQ ID NO: 39) |
| | TAA CAC TCG TCA ATC | |
| | CCC AGC TCG CAG GTG | |

PCR was carried out using Pfu turbo polymerase (Stratagene, La Jolla, Calif., US) with cycling conditions as follows: 95 C 5 min, 95 C 1 min, 45-69 C 1 min, 72 C 1 min for 25 cycles, 72 C 10 min.

The products at 58 C, 62 C & 67 C were purified from 1% agarose gel in 1× TAE using a Qiagen gel extraction kit according to the manufacturer's instructions, ligated into pCRIIblunt vector (InVitrogen TOPO-blunt kit) and then transformed into TOP10 cells (InVitrogen). The resulting clone sequence was verified, and only the original two silent mutations were found to be present in the parental clone.

The resulting sequence coding for “D1E3Cys” was excised using PmeI and HindIII, purified on 1% agarose gel, 1× TAE using a Qiagen gel extraction kit and ligated into pCDNA3.1V5HIS (Invitrogen) between the PmeI and HindIII sites, thereby eliminating the V5HIS sequence. The resulting DNA was transformed into TOP10 cells. The resulting clone sequence was verified at the 3′-ligation site.

The D1E3Cys-coding fragment was excised from the pCDNA3.1 plasmid using PmeI and HindIII. A pEE14.4 vector plasmid (Lonza Biologics, UK) was then restricted using EcoRI, and the 5′-overhangs were filled in using Klenow fragment polymerase. The vector DNA was cleaned on a Qiagen PCR purification column, restricted using HindIII, then treated with Shrimp Alkaline Phosphatase (Roche). The pEE14.4 vector and D1E3cys fragments were purified on 1% agarose gel in 1× TAE using a Qiagen gel extraction kit prior to ligation (T4 ligase) to give plasmid pEE14.4 DLLΔ4-8cys. The resulting clone sequence was verified.

The D1E3Cys coding sequence is as follows (SEQ ID NO:40):

| | |
|---|---|
| 1 | atgggcagtc ggtgcgcgct ggccctggcg gtgctctcgg |
| | ccttgctgtg |
| 51 | tcaggtctgg agctctgggg tgttcgaact gaagctgcag |
| | gagttcgtca |
| 101 | acaagaaggg gctgctgggg aaccgcaact gctgccgcgg |
| | gggcgcgggg |
| 151 | ccaccgccgt gcgcctgccg gaccttcttc cgcgtgtgcc |
| | tcaagcacta |
| 201 | ccaggccagc gtgtcccccg agccgccctg cacctacggc |
| | agcgccgtca |
| 251 | cccccgtgct gggcgtcgac tccttcagtc tgcccgacgg |
| | cgggggcgcc |
| 301 | gactccgcgt tcagcaaccc catccgcttc cccttcggct |
| | tcacctggcc |
| 351 | gggcaccttc tctctgatta ttgaagctct ccacacagat |
| | tctcctgatg |
| 401 | acctcgcaac agaaaaccca gaaagactca tcagccgcct |
| | ggccacccag |
| 451 | aggcacctga cggtgggcga ggagtggtcc caggacctgc |
| | acagcagcgg |
| 501 | ccgcacggac ctcaagtact cctaccgctt cgtgtgtgac |
| | gaacactact |
| 551 | acggagaggg ctgctccgtt ttctgccgtc cccgggacga |
| | tgccttcggc |
| 601 | cacttcacct gtggggagcg tggggagaaa gtgtgcaacc |
| | ctggctggaa |
| 651 | agggccctac tgcacagagc cgatctgcct gcctggatgt |
| | gatgagcagc |
| 701 | atggattttg tgacaaacca ggggaatgca agtgcagagt |
| | gggctggcag |
| 751 | ggccggtact gtgacgagtg tatccgctat ccaggctgtc |
| | tccatggcac |
| 801 | ctgccagcag ccctggcagt gcaactgcca ggaaggctgg |
| | gggggccttt |
| 851 | tctgcaacca ggacctgaac tactgcacac accataagcc |
| | ctgcaagaat |
| 901 | ggagccacct gcaccaacac gggccagggg agctacactt |
| | gctcttgccg |
| 951 | gcctgggtac acaggtgcca cctgcgagct ggggattgac |
| | gagtgttaa |

The DNA was prepared for stable cell line transfection/selection in a Lonza GS system using a Qiagen endofree maxi-prep kit.

ii) Expression of D1E3Cys

Linearisation of DNA

The pEE14.4 DLLΔ4-8cys plasmid DNA from (i) above was linearised by restriction enzyme digestion with PvuI, and then cleaned up using phenol chloroform isoamyl alcohol (IAA), followed by ethanol precipitation. Plasmid DNA was checked on an agarose gel for linearisation, and spec'd at 260/280 nm for quantity and quality of prep.

Transfection

CHO-K1 cells were seeded into 6 wells at 7.5×10⁵ cells per well in 3 ml media (DMEM 10% FCS) 24 hrs prior to transfection, giving 95% confluency on the day of transfection. Lipofectamine 2000 was used to transfect the cells using 5 ug of linearised DNA. The transfection mix was left on the cell sheet for 5½ hours before replacing with 3 ml semi-selective media (DMEM, 10% dFCS, GS) for overnight incubation.

At 24 hours post-transfection the media was changed to full selective media (DMEM (Dulbecco's Modified Eagle Medium), 10% dFCS (fetal calf serum), GS (glutamine synthase), 25 uM L-MSX (methionine sulphoximine)) and incubated further.

Cells were plated into 96 wells at 10⁵ cells per well on days 4 and 15 after transfection.

96 well plates were screened under a microscope for growth 2 weeks post clonal plating. Single colonies were identified and scored for % confluency. When colony size was >30% media was removed and screened for expression by dot blot against anti-human-Delta-1 antisera. High positives were confirmed by the presence of a 36kDa band reactive to anti-human-Delta-1 antisera in PAGE Western blot of media.

Cells were expanded by passaging from 96 well to 6 well to T25 flask before freezing. The fastest growing positive clone (LC09 0001) was expanded for protein expression.

D1E3Cys Expression and Purification

T500 flasks were seeded with 1×10⁷ cells in 80 ml of selective media. After 4 days incubation the media was removed, cell sheet rinsed with DPBS and 150 ml of 325 media with GS supplement added to each flask. Flasks were incubated for 7 further days before harvesting. Harvest media was filtered through a 0.65-0.45 um filter to clarify prior to freezing. Frozen harvests were purified by FPLC as follows:

Frozen harvest was thawed and filtered. A 17 ml Q Sepharose column was equilibrated in 0.1M Tris pH 8 buffer, for 10 column volumes. The harvest was loaded onto the column using a P1 pump set at 3 ml/min, the flowthrough was collected into a separate container (this is a reverse purification—a lot of the BSA contaminant binds to the Q Sepharose FF and our target protein does not and hence remains in the flowthrough). The flowthrough was concentrated in a TFF rig using a 10 kDa cut off filter cartridge, during concentration it was washed 3× with 0.1M Sodium phosphate pH 7 buffer. The 500 ml was concentrated down to 35 ml, to a final concentration of 3 mg/ml.

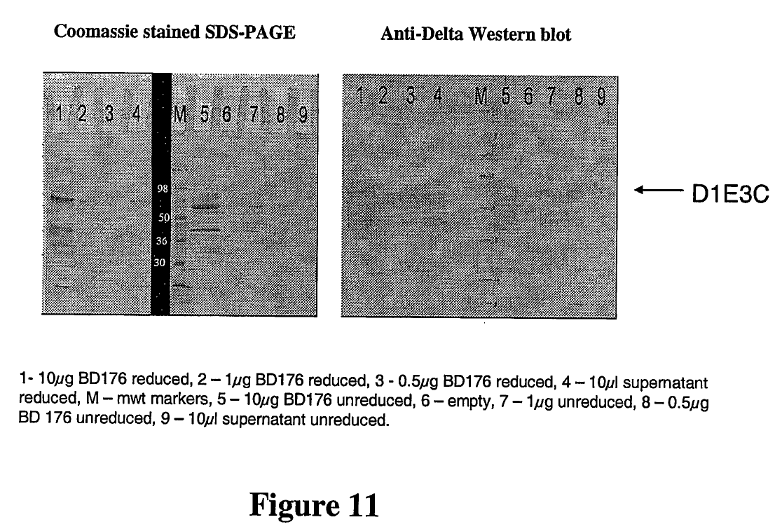

Samples were run on SDS PAGE reduced and non-reduced (gels are shown in FIG. 11)

The amino acid sequence of the resulting expressed D1E3Cys protein was as follows (SEQ ID NO:41):

MGSRCALALAVLSALLCQVWSSGVFELKLQEFVNKKGLLGNRNCCRGGAGPPPCACRTF
FRVCLKHYQASVSPEPPCTYGSAVTPVLGVDSFSLPDGGGADSAFSNPIRFPFGFTWPG
TFSLIIEALHTDSPDDLATENPERLISRLATQRHLTVGEEWSQDLHSSGRTDLKYSYRF
VCDEHYYGEGCSVFCRPRDDAFGHFTCGERGEKVCNPGWKGPYCTEPICLPGCDEQHGF
CDKPGECKCRVGWQGRYCDECIRYPGCLHGTCQQPWQCNCQEGWGGLFCNQDLNYCTHH
KPCKNGATCTNTGQGSYTCSCRPGYTGATCELGIDE+E,usn C

(wherein the sequence in italics is the leader peptide, the underlined sequence is the DSL domain, the bold sequences are the three EGF repeats, and the terminal Cys residue is shown bold underlined).
iii) Reduction of D1E3cys Protein

40 μg D1E3Cys protein from (ii) above was made up to 100 μl to include 100 mM sodium phosphate pH 7.0 and 5 mM EDTA. 2 volumes of immobilised TCEP (tris[2-carboxyethyl]phosphine hydrochloride; Pierce, Rockford, Ill., US, Cat No: 77712; previously washed 3 times 1 ml 100 mM sodium phosphate pH 7.0) were added and the mixture was incubated for 30 minutes at room temperature, with rotating.

The resin was pelleted at room temperature in a microfuge (13,000 revs/min, 5 minutes) and the supernatant was transferred to a clean Eppendorf tube and stored on ice. Protein concentration was measured by Warburg-Christian method.

This fragment is linked to a polymer such as dextran or PEG as described above to provide the final conjugate.

### Example 5

### Coupling of D1 E3cys to Amino-Dextran to Provide Conjugate

i) Purification of Expressed D1E3Cys by HIC

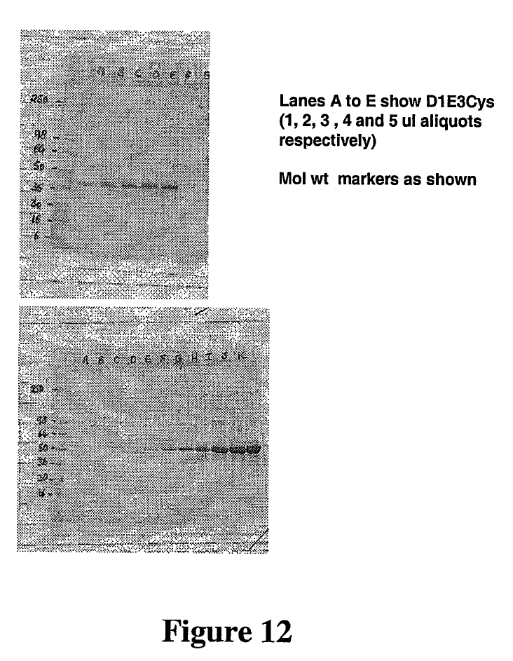

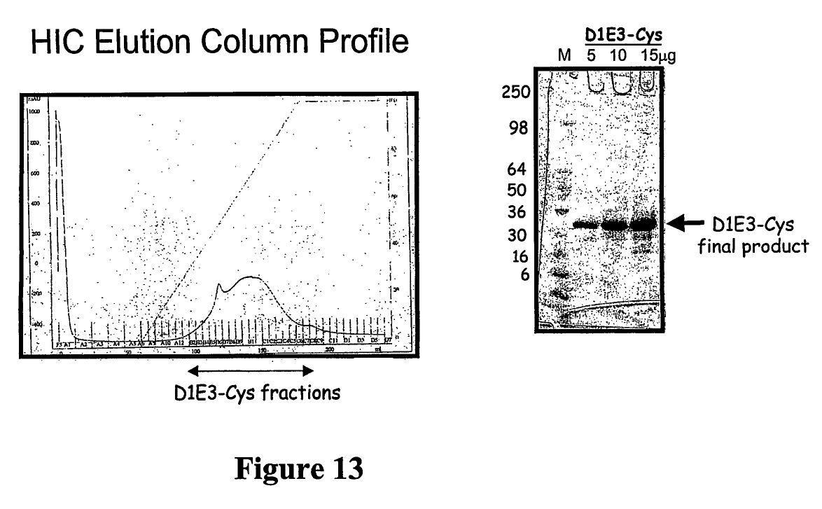

Harvests from Example 4 above were purified using Hydrophobic Interaction Chromatography (HIC), the eluate was then concentrated and buffer exchanged using centrifugal concentrators according to the manufacturers' instructions. The purity of the product was determined by SDS PAGE. Sample gels are shown in FIG. 12 and a sample gel and purification trace is shown in FIG. 13.

ii) Maleimide Substitution of Amino-Dextran (Polymer Activation)

Amino-dextran of molecular mass 500,000 Da (dextran, amino, 98 moles amine/mole; Molecular Probes, ref D-7144), 3.2 mg/ml, was derivatised/activated with sulfo-SMCC (sulfosuccinimidyl 4-[N-maleimidomethyl]-cyclohexane-1-carboxylate; Pierce, ref 22322) at 73 moles sulfo-SMCC per mole amino-dextran in 100 mM sodium phosphate pH 8.0 for 1 h, 22° C.

The amino content of the dextran and the level of maleimide substitution was measured using a Ninhydrin assay. Aliquots of dextran derivative or B-alanine (Sigma, A-7752) were made to 50 μl in 100 mM sodium phosphate pH 7.0 and diluted in water to 250 μl. Ninhydrin reagent solution (Sigma, N1632) was added, 1 vol., and samples heated 100° C., 15 min. After cooling on ice 1 vol. 50% ethanol was added, mixed and the solution clarified by centrifugation. Absorbance was recorded at 570 nm.

The resulting maleimido-dextran was purified and concentrated by buffer exchange using Vivaspin 6 ml concentrators (VivaScience, VS0612) and 3×5 ml, 100 mM sodium phosphate pH 7.0.

The concentration of dextran was measured using an ethanol precipitation/turbidity assay. Aliqouts of dextran derivative were made to 50 μl in 100 mM sodium phosphate pH 7.0. Water was added to make 500 μl final volume, dextran was precipitated by the addition of 1 vol. absolute ethanol and absorbance was recorded at 600 nm.

iii) Partial Reduction of D1E3cys

D1E3cys protein (purified as in (i) above) at 1 mg/ml in 100 mM sodium phosphate pH 7.0 was reduced using TCEP.HCl (Tris(2-carboxyethyl)phosphine hydrochloride; Pierce, 20490) at a 10-fold molar excess of reducing agent for 1 h at 22° C. The protein was purified by buffer exchange using Sephadex G-25, PD-10 columns (Amersham biosciences, 17-0851-01) into 100 mM sodium phosphate pH 7.0 followed by concentration in Vivaspin 6 ml concentrators. Protein concentration was estimated using the Warburg-Christian A280/A260 method.

The efficiency of reduction can be estimated using the Ellman's assay. The supplied D1E3cys protein has no free thiol groups, whereas partially reduced D1E3cys is predicted to have a single free thiol group per mole of protein. Using a 96-well microtitre plate, aliqouts of D1E3cys protein or L-cysteine hydrochloride (Sigma, C-1 276) were made to 196 ul in 100 mM sodium phosphate pH 7.0 and 4 ul 4 mg/ml Ellman's reagent (in 100 mM sodium phosphate pH 7.0) was added. Reactions were incubated for 15 min at 22° C. and absorbance was recorded at 405 nm.

iv) Coupling of Reduced D1E3cys to Maleimido-Dextran.

The derivatized maleimido-dextran was added to concentrated, reduced D1E3cys at a 1:75 molar ratio of dextran to D1E3cys. Coupling proceeded for 18 h, 4° C.

The resulting D1E3cys-dextran polymer (D1E3Cys-dextran conjugate; comprising aminodextrans each coupled to a large number of D1E3Cys proteins via SMCC linkers) was purified by gel permeation chromatography using a Superdex 200 (Amersham Biosciences, 17-1043-10) column attached to an AKTA purifier FPLC (Amersham Biosciences) in 100 mM sodium phosphate pH 7.0. At a flow rate of 1 ml/min, 1 ml fractions were collected. The protein complex was then concentrated in Vivaspin 6 ml concentrators and protein concentration was measured using the Warburg-Christian A280/A260 method.

The complex was analysed on SDS-PAGE gel and screened for endotoxin contamination prior to activity assays in vitro and in vivo as described below.

### Example 6

### CHO-N2 (N27) Luciferase Reporter Assay

A) Construction of Luciferase Reporter Plasmid 10×CBF1-Luc (pLOR91)

As described in WO 03/012441 an adenovirus major late promoter TATA-box motif with BglII and HindIII cohesive ends was generated as follows:

BglII HindIII
GATCTGGGGGGCTATAAAAGGGGGTA
ACCCCCCGATATTTTCCCCCATTCGA
(SEQ ID NOS: 42 and 43)

This was cloned into plasmid pGL3-Basic (Promega) between the BgiII and HindIII sites to generate plasmid pGL3-AdTATA.

A TP1 promoter sequence (TP1; equivalent to 2 CBF1 repeats) with BamH1 and BglII cohesive ends was generated as follows:

BamH1 BglII
5′ GATCCCGACTCGTGGGAAAATGGGCGGAAGGGCACCGTGGGAAAATAGTA 3′
3′ GGCTGAGCACCCTTTTACCCGCCTTCCCGTGGCACCCTTTTATCATCTAG 5′
(SEQ ID NOS: 44 and 45)

This sequence was pentamerised by repeated insertion into a BglII site and the resulting TP1 pentamer (equivalent to 10 CBF1 repeats) was inserted into pGL3-AdTATA at the BglII site to generate plasmid pLOR91.

B) Generation of a Stable CHO Cell Reporter Cell Line Expressing Full Length Notch2 and the 10×CBF1-Luc Reporter Cassette

A cDNA clone spanning the complete coding sequence of the human Notch2 gene (see, eg GenBank Accession No AF315356) was constructed as follows. A 3′ cDNA fragment encoding the entire intracellular domain and a portion of the extracellular domain was isolated from a human placental cDNA library (OriGene Technologies Ltd., USA) using a PCR-based screening strategy. The remaining 5′ coding sequence was isolated using a RACE (Rapid Amplification of cDNA Ends) strategy and ligated onto the existing 3′ fragment using a unique restriction site common to both fragments (Cla I). The resulting full-length cDNA was then cloned into the mammalian expression vector pcDNA3.1-V5-HisA (Invitrogen) without a stop codon to generate plasmid pLOR92: When expressed in mammalian cells, pLOR92 thus expresses the full-length human Notch2 protein with V5 and His tags at the 3′ end of the intracellular domain.

Wild-type CHO-K1 cells (eg see ATCC No CCL 61) were transfected with pLOR92 (pcDNA3.1-FLNotch2-V5-His) using Lipfectamine 2000™ (Invitrogen) to generate a stable CHO cell clone expressing full length human Notch2 (N2). Transfectant clones were selected in Dulbecco's Modified Eagle Medium (DMEM) plus 10% heat inactivated fetal calf serum ((HI)FCS) plus glutamine plus Penicillin-Streptomycin (P/S) plus 1 mg/ml G418 (Geneticin™—Invitrogen) in 96-well plates using limiting dilution. Individual colonies were expanded in DMEM plus 10% (HI)FCS plus glutamine plus P/S plus 0.5 mg/ml G418. Clones were tested for expression of N2 by Western blots of cell lysates using an anti-V5 monoclonal antibody (Invitrogen). Positive clones were then tested by transient transfection with the reporter vector pLOR91 (10×CBF1-Luc) and co-culture with a stable CHO cell clone (CHO-Delta) expressing full length human delta-like ligand 1 (DLL1; eg see GenBank Accession No AF196571). CHO-Delta cells were prepared in the same way as the CHO Notch 2 clone, but with human DLL1 used in place of Notch 2. A strongly positive clone was selected by Western blots of cell lysates with anti-V5 mAb.

One CHO-N2 stable clone, N27, was found to give high levels of induction when transiently transfected with pLOR91 (10×CBF1-Luc) and co-cultured with the stable CHO cell clone expressing full length human DLL1 (CHO-Delta1). A hygromycin gene cassette (obtainable from pcDNA3.1/hygro, Invitrogen) was inserted into pLOR91 (10×CBF1-Luc) using BamH1 and Sal1 and this vector (10×CBF1-Luc-hygro) was transfected into the CHO-N2 stable clone (N27) using Lipfectamine 2000 (Invitrogen). Transfectant clones were selected in DMEM plus 10% (HI)FCS plus glutamine plus P/S plus 0.4 mg/ml hygromycin B (Invitrogen) plus 0.5 mg/ml G418 (Invitrogen) in 96-well plates using limiting dilution. Individual colonies were expanded in DMEM plus 10% (HI)FCS plus glutamine plus P/S+0.2 mg/ml hygromycin B plus 0.5 mg/ml G418 (Invitrogen).

Clones were tested by co-culture with a CHO Delta (expressing full length human Delta1 (DLL1)). Three stable reporter cell lines were produced N27#11, N27#17 and N27#36. N27#11 was selected for further use because of its low background signal in the absence of Notch signalling, and hence high fold induction when signalling is initiated. Assays were set up in 96-well plates with 2×10⁴ N27#11 cells per well in 100 μl per well of DMEM plus 10% (HI)FCS plus glutamine plus P/S.

CHO-Delta cells (as described above) were maintained in DMEM plus 10% (HI)FCS plus glutamine plus P/S plus 0.5 mg/ml G418. Just prior to use the cells were removed from a T80 flask using 0.02% EDTA solution (Sigma), spun down and resuspended in 10 ml DMEM plus 10% (HI)FCS plus glutamine plus P/S. 10 μl of cells were counted and the cell density was adjusted to 5.0×10⁵ cells/ml with fresh DMEM plus 10% (HI)FCS plus glutamine plus P/S.

To set up the CHO-Delta assay, N27#11 cells (T₈₀ flask) were removed using 0.02% EDTA solution (Sigma), spun down and resuspended in 10 ml DMEM plus 10% (HI)FCS plus glutamine plus P/S. 10 μl of cells were counted and the cell density was adjusted to 2.0×10⁵ cells/ml with fresh DMEM plus 10% (HI)FCS plus glutamine plus P/S. The reporter cells were plated out at 100 μl per well of a 96-well plate (i.e. 2×10⁴ cells per well) and were placed in an incubator to settle down for at least 30 minutes.

D1E3Cys conjugates prepared as described above were diluted in PBS (20 ug/ml) and added to eg 100 μl of N27#11 cells in a 96-well plate. Plates were placed at 37° C. in an incubator, suitably overnight.

The following day 150 μl of supernatant was removed from all the wells, 100 μl of SteadyGlo™ luciferase assay reagent (Promega) was added and the resulting mixture left at room temperature for 5 minutes. The mixture was then pipetted up and down 2 times to ensure cell lysis and the contents from each well are transferred to a white 96-well plate (Nunc). Luminescence is then read in a TopCount™ (Packard) counter. An increase in luminescence compared to control indicates activation of Notch signalling (ie an active conjugate).

In a variation on the above, (allowing for plate binding) the dextran-D1E3 Cys conjugate was added to the plates prior to cell loading at concentrations of up to 250 ug/ml in PBS (pre-addition) and the mixture was incubated overnight before conducting a luciferase assay as described above.

CHO cells expressing full length human Delta1 (CHO-Delta cells; prepared as described in WO 03/0102441 in the name of Lorantis Ltd; eg see Example 8 therein) and native CHO cells were used as controls at a cell ratio of 1:1 to the reporter cells.

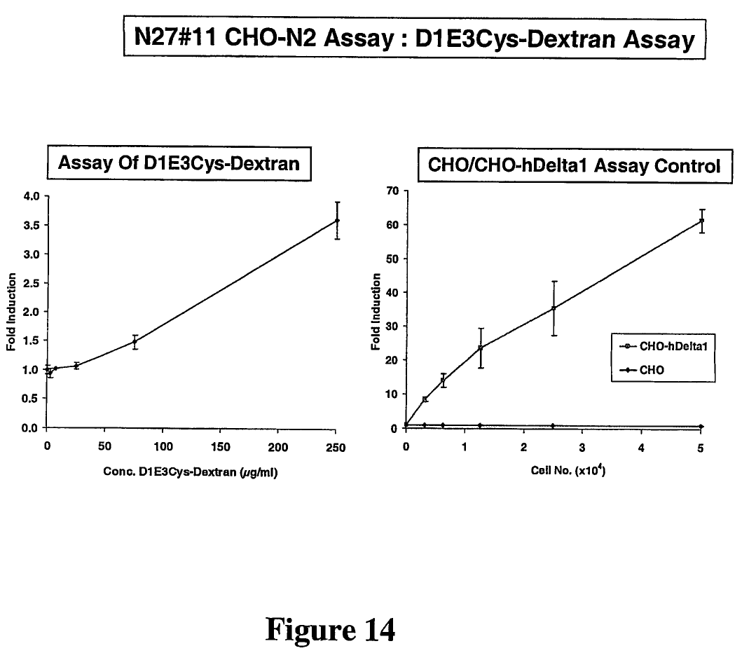

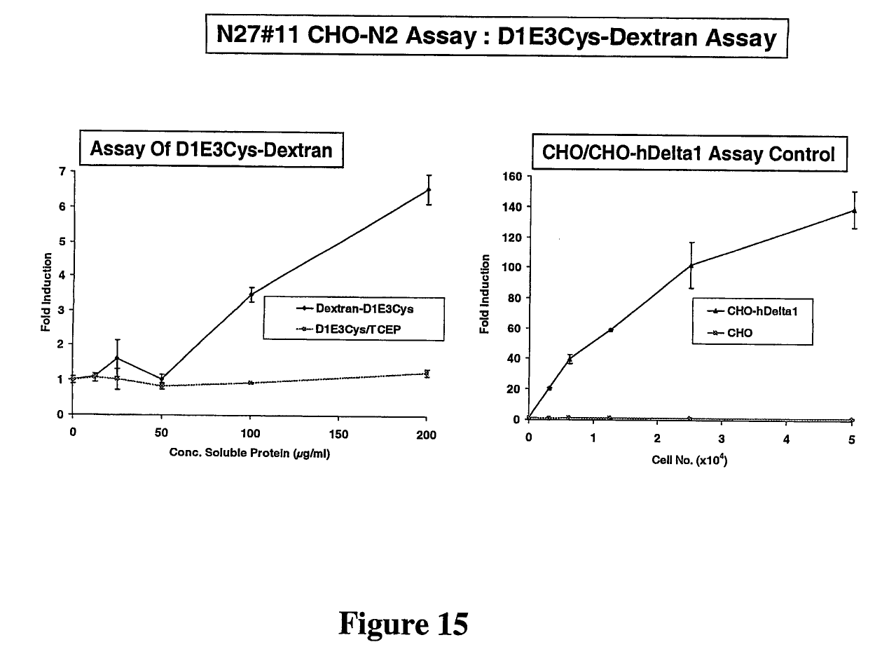

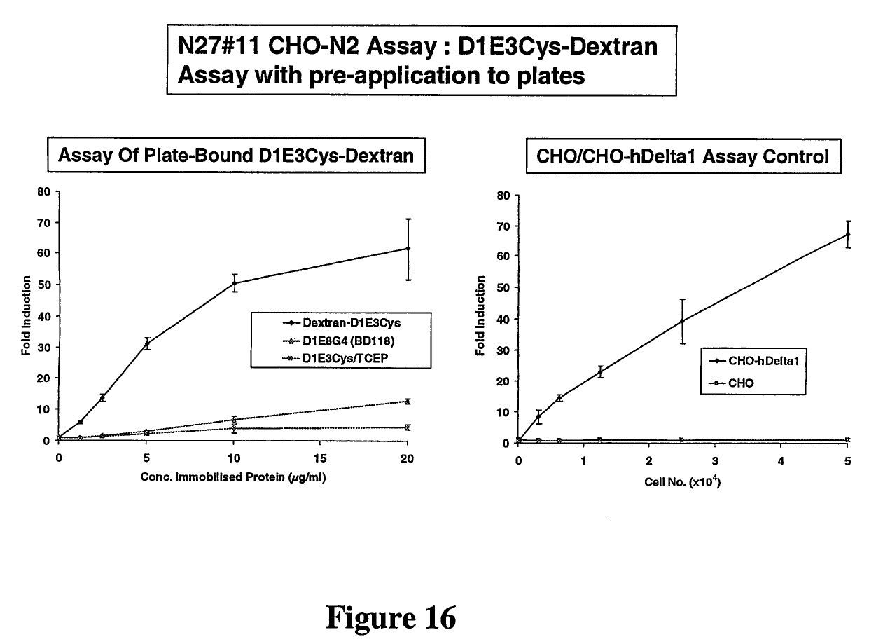

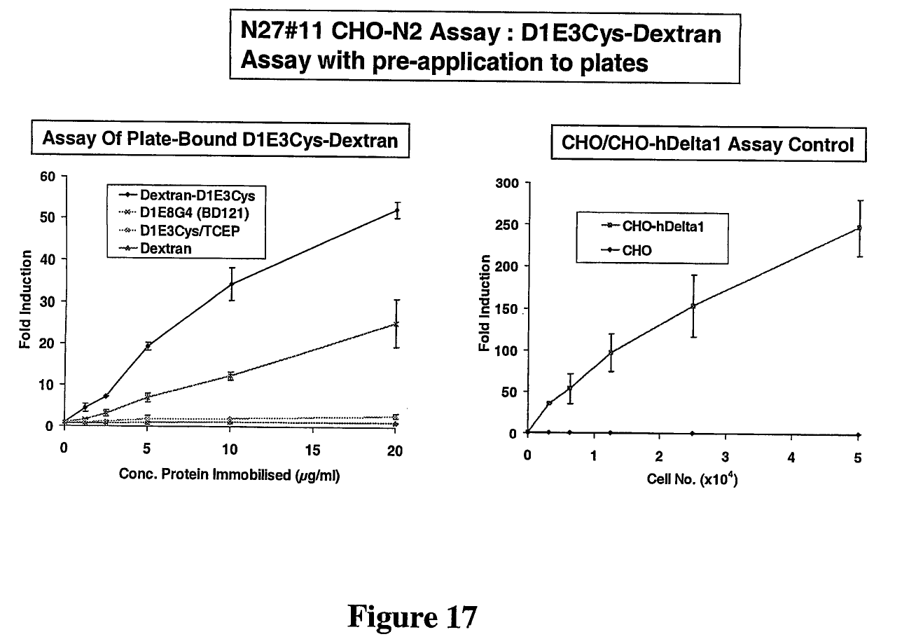

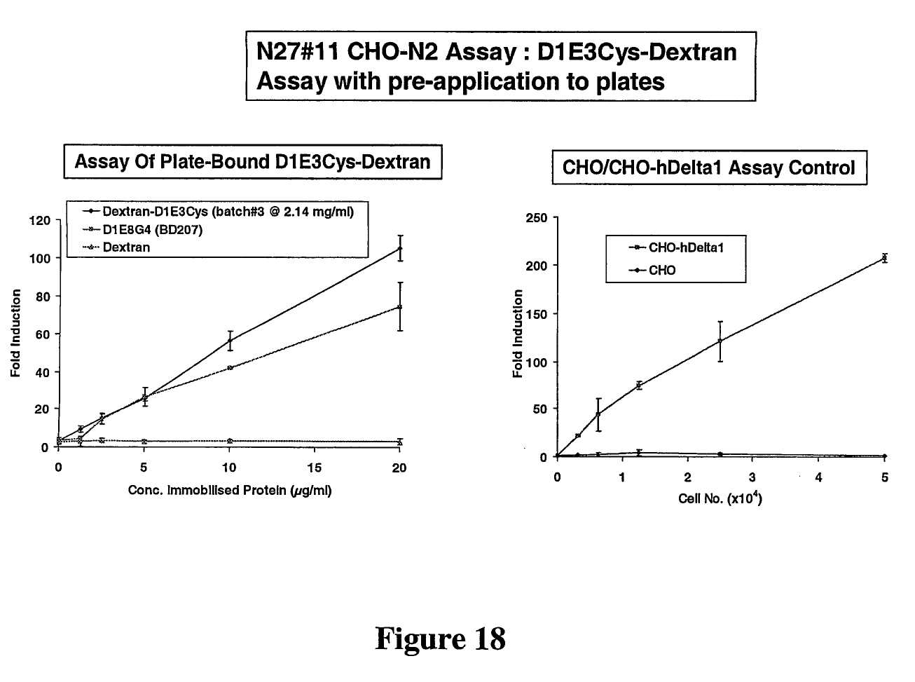

Results are shown in FIGS. 14-18 alongside the corresponding CHO/CHO-Delta controls. FIGS. 14 and 15 show results obtained without pre-addition of the conjugate to the plates (ie with conjugate added at the same time as the cells/FCS) and FIGS. 16 to 18 show results obtained with pre-addition of the conjugate (ie with conjugate added to plates the day before the cells/FCS to facilitate binding of conjugate to the plate).

### Example 7

### Immune Cell Assays

i) CD4+ Cell Purification

Spleens were removed from mice (Balb/c females, 8-10 weeks) and treated with 1 mg/ml Collagenase D (Boehringer Mannheim) in RPMI medium with no supplements for 40 min. Tissue was passed through a 70 um cell strainer (Falcon) into 20 ml R10F medium [R10F-RPMI 1640 medium (Gibco Cat No 22409) plus 2 mM L-glutamine, 50 μg/mL penicillin, 50 μg/mL streptomycin, 5×10⁻⁵ M β-mercapoethanol and 10% fetal calf serum]. The cell suspension was centrifuged (1140 rpm, 6 min) and the medium removed.

The cells were then incubated at room temperature for 5 min with 5 mL ACK lysis buffer (0.15M NH₄Cl, 1.0M KHCO₃, 0.1 mM Na₂EDTA in double distilled water) per spleen to lyse red blood cells. The cells were washed once with R10F medium and counted. CD4+ cells were purified from the suspensions by positive selection on a Magnetic Associated Cells Sorter (MACS) column (Miltenyi Biotech, Bisley, UK, Cat No 130-042-401) using CD4 (L3T4) beads (Miltenyi Biotech Cat No 130-049-201), according to the manufacturer's instructions.

ii) Antibody Coating

The following protocol was used for coating 96 well flat-bottomed tissue culture plates with antibodies:

Dulbecco's Phosphate Buffered Saline (DPBS) plus 1 μg/mL anti-hamster IgG (Pharmingen, San Diego, US: Cat No 554007)±1μg/mL anti-human IgG4 (Pharmingen: Cat No 555878) was added at 100 μL per well. Plates were incubated for 3-6 hr at 37° C. then washed with DPBS. Each well then received 100 μL DPBS plus 0.1-1 μg/mL anti-CD3 (Pharmingen Cat No 553058, Clone No 145-2C11)±Notch ligand (hDelta1-hIgG4 or D1E3 Cys) or control protein (huIgG4, Sigma: Cat No I-4639).

The plates were incubated overnight at 4° C. then washed again with DPBS.

In some cases, before cells (prepared as above) were added, plates were blocked by addition of 200 uL DPBS containing 1-5% foetal bovine serum +50 ug/mL aminodextran for 3-6 hr at 37° C. and washed in DPBS.

iii) Primary Polyclonal Stimulation and Cytokine ELISA

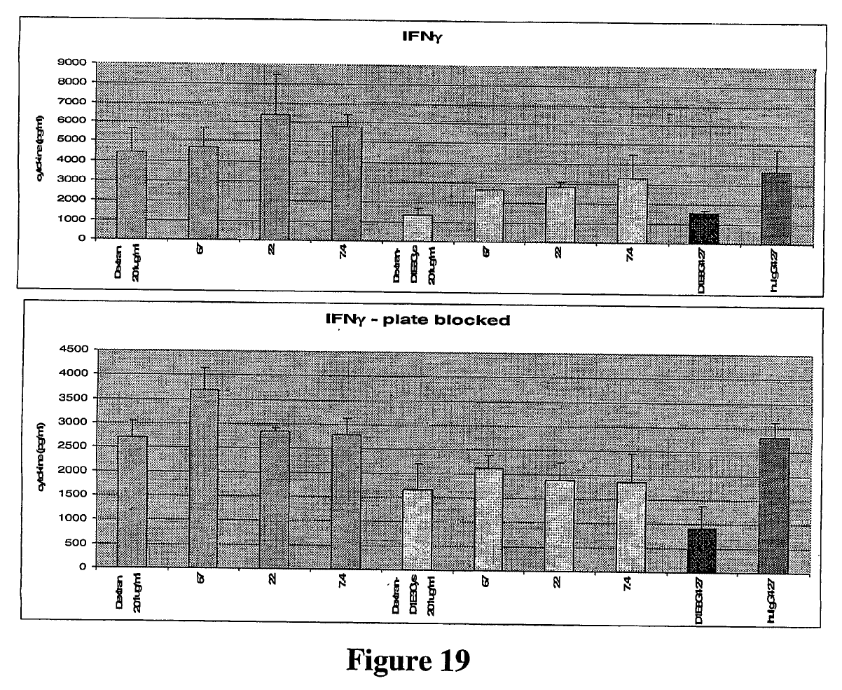

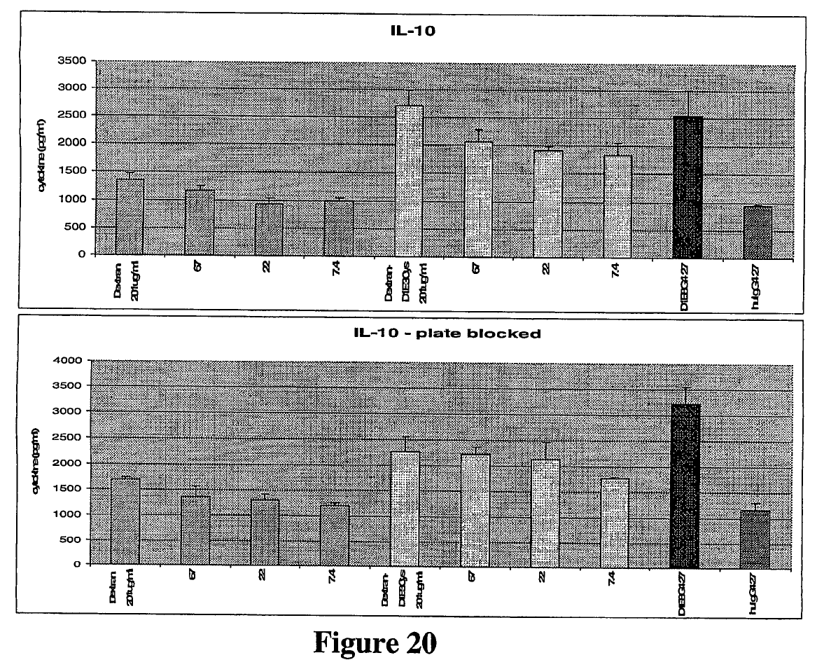

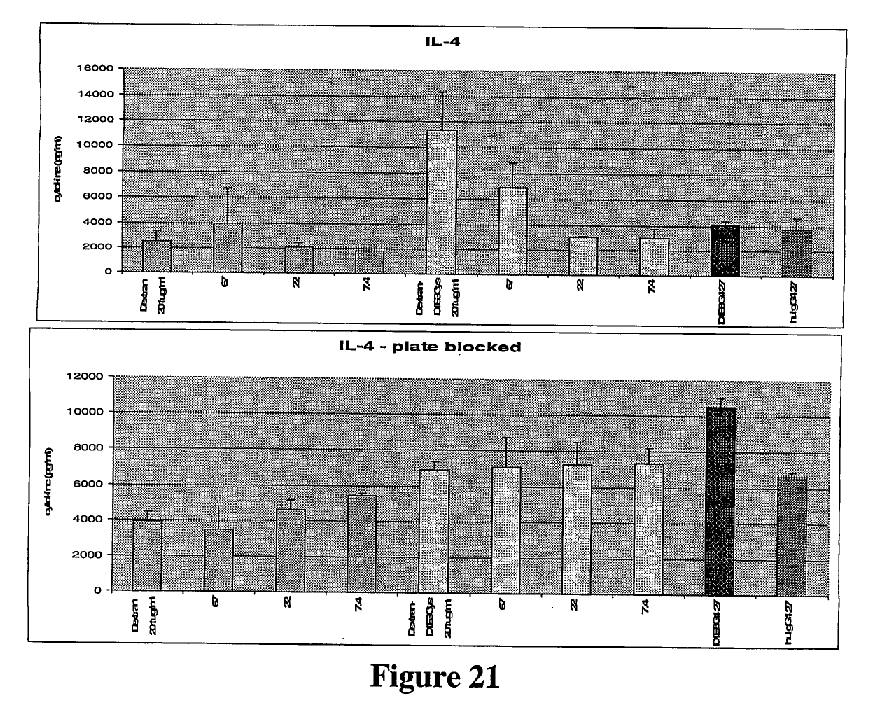

CD4+ cells were cultured in 96 well, flat-bottomed plates pre-coated as above. Cells were resuspended following counting at 2×10⁶/mL in R10F medium plus 4 μg/mL CD28 antibody (Pharmingen, Cat No 553294, Clone No 37.51) and 100 μL suspension added per well. Dextran multimerised with Notch ligand (D1E3 Cys-dextran conjugate; from Example 5 above) was added in 100 uL RPMI medium at appropriate concentrations to give final concentrations of 1-250 ug/mL, to give a final volume of 200 μL per well (2×10⁵ cells/well, anti-CD28 final concentration 2 μg/mL). The plates were then incubated at 37° C. for 72 hours. 170 μL supernatant was then removed from each well and stored at −20° C. until tested by ELISA for IL-10, IFNγ and IL-4 using antibody pairs from R&D Systems (Abingdon, UK). Results (with plates either blocked or unblocked as indicated) are shown in FIGS. 19 to 21.

### Example 8

### Co-Administration of KLH Beads and Dextran-D1E3cys Conjugate in vivo

i) Coating of Beads with KLH

Imject® Mariculture Keyhole Limpet Hemocyanin (mcKLH) in PBS Buffer (lyophilized from PBS) 20 mg (Pierce product number 77600) was reconstituted with 2.0 ml dH₂O to make a 10 mg/ml solution containing PBS, pH 7.2 with proprietary stabilizer.

Surfactant-free White Aldehyde/Sulfate Latex Beads (Interfacial Dynamics corp Portland or USA batch number 1813) concentration 5.8×10⁸ beads/ml were washed in PBS×3 (spun for 10 mins at 13 k RT). The beads were then resuspended at 2×10⁸ beads/ml in 500 μg/ml mcKLH in PBS and horizontally rotated at 37° C. overnight. Beads were then washed again in PBS×3 (spun for 10 mins at 13 k RT) and resuspended in PBS at the required concentration. Successful coating of the beads was checked by their ability to neutralize an anti-KLH antiserum in an ELISA system.

ii) In Vivo Administration with D1E3Cys/Dextran Conjugate

6-8 weeks old female Balb/c mice were injected s.c. at the base of the tail with 2×10⁶ KLH coated beads (prepared as described in (i) above) per mouse. Dextran-D1E3cys conjugate from Example 5 above (250, 50 or 10 82 g per mouse), D1E3 Cys alone (control) or dextran alone (control) were injected s.c. in a close separate site of the tail base (all agents were administered as aqueous solutions; 100 mM sodium phosphate at pH 7).

Mice were challenged after 7 days in the right ear with 20 μg of KLH. The increase in ear swelling (right ear—left ear) was measured for the following four days using a digital calliper.

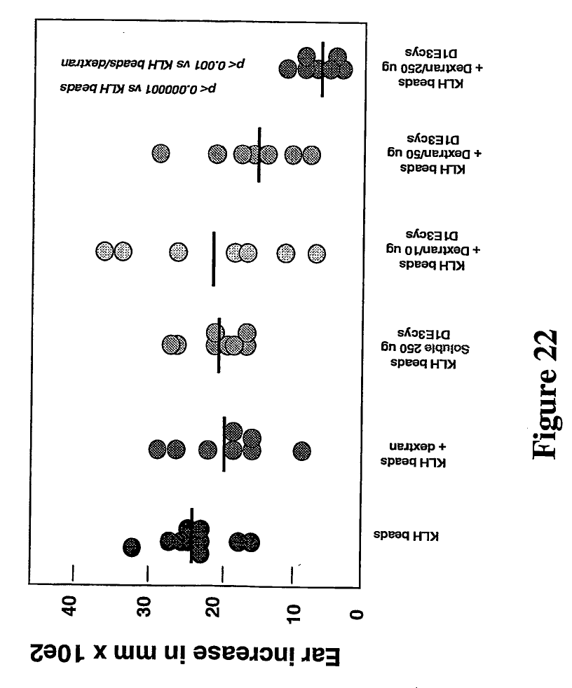

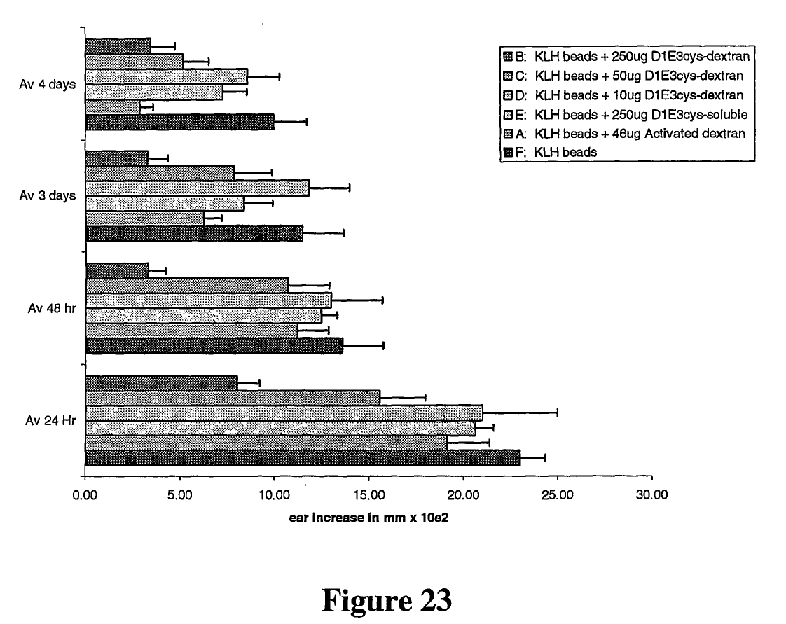

Results are shown in FIGS. 22 and 23. As can be seen from these Figures, the control groups (KLH beads, KLH beads plus dextran alone and KLH beads plus soluble D1E3Cys alone) showed a similar degree of response at 24 hours and 48 hours. KLH beads plus D1E3cys/dextran conjugate 250 μg showed a significant decreased DTH response at 24 hours and 48 hours (p<0.001 vs KLH beads plus dextran alone).

The invention is further described in the following numbered paragraphs.

1. A conjugate comprising a plurality of modulators of the Notch signalling pathway chemically bound to a support structure.

2. A conjugate comprising a plurality of modulators of the Notch signalling pathway chemically bound to a molecular support structure.

3. A conjugate as described in paragraph 1 or paragraph 2 wherein the support structure has a molecular weight of between about 500 and about 100,000 Da.

4. A conjugate as described in paragraph 3 wherein the support structure has a molecular weight of between about 1000 and about 50,000 Da.

5. A conjugate as described in any one of the preceding paragraphs wherein the support structure comprises a polymeric material.

6. A conjugate as described in paragraph 5 wherein the polymeric material comprises polyethylene glycol or a residue thereof.

7. A conjugate as described in paragraph 6 wherein the polymeric material comprises a branched chain polyethylene glycol polymer or a residue thereof.

8. A conjugate as described in any one of paragraphs wherein at least one of the modulators of the Notch signalling pathway is coupled to the support structure via a linker moiety.

9. A conjugate as described in paragraph 8 wherein the linker comprises an acid, basic, aldehyde, ether or ester reactive group or a residue thereof.

10. A conjugate as described in paragraph 9 wherein the linker moiety is a succinimidyl propionate, succinimidyl butanoate, N-hydroxysuccinimide, benzotriazole carbonate, propionaldehyde, maleimide or forked maleimide, biotin, vinyl derivative or phospholipid.

11. A conjugate comprising a plurality of modulators of the Notch signalling pathway in chemically cross-linked form.

12. A conjugate as described in any one of the preceding paragraphs comprising at least three modulators of the Notch signalling pathway.

13. A conjugate as described in any paragraph 12 comprising at least four modulators of the Notch signalling pathway.

14. A conjugate as described in paragraph 13 comprising at least five modulators of the Notch signalling pathway.

15. A conjugate as described in paragraph 13 comprising at least 10 modulators of the Notch signalling pathway.

16. A conjugate as described in paragraph 13 comprising at least 20 modulators of the Notch signalling pathway.

17. A conjugate as described in paragraph 13 comprising at least 30 modulators of the Notch signalling pathway.

18. A conjugate as described in any one of the preceding paragraphs wherein at least one of the modulators of the Notch signalling pathway is an agent capable of activating a Notch receptor.

19. A conjugate as described in any one of the preceding paragraphs wherein at least one of the modulators of the Notch signalling pathway comprises a Notch ligand or a fragment, derivative, homologue, analogue or allelic variant thereof.

20. A conjugate as described in any one of the preceding paragraphs wherein at least one of the modulators of the Notch signalling pathway comprises a Delta or Serrate/Jagged protein or a fragment, derivative, homologue, analogue or allelic variant thereof.

21. A conjugate as described in any one of the preceding paragraphs wherein at least one of the modulators of the Notch signalling pathway comprises a fusion protein comprising a segment of a Notch ligand extracellular domain and an immunoglobulin Fc segment.

22. A conjugate as described in any one of the preceding paragraphs wherein at least one of the modulators of the Notch signalling pathway comprises a protein or polypeptide comprising a DSL or EGF-like domain or a fragment, derivative, homologue, analogue or allelic variant thereof.

23. A conjugate as described in any one of the preceding paragraphs wherein at least one of the modulators of the Notch signalling pathway comprises a protein or polypeptide comprising at least one Notch ligand DSL domain and at least 1 Notch ligand EGF domain.

24. A conjugate as described in any one of the preceding paragraphs wherein at least one of the modulators of the Notch signalling pathway comprises a protein or polypeptide comprising at least one Notch ligand DSL domain and at least 2 Notch ligand EGF domains.

25. A conjugate as described in any one of the preceding paragraphs wherein at least one of the modulators of the Notch signalling pathway comprises a protein or polypeptide comprising at least one Notch ligand DSL domain and at least 3 Notch ligand EGF domains.

26. A conjugate as described in any one of paragraphs 1 to 25 comprising a modulator of Notch signalling consisting essentially of the following components:

i) a Notch ligand DSL domain;

ii) 1-5 and no more than 5 Notch ligand EGF domains;

iii) optionally all or part of a Notch ligand N-terminal domain; and

iv) optionally one or more heterologous amino acid sequences.

27. A conjugate as described in any one of paragraphs 1 to 25 comprising a modulator of Notch signalling consisting essentially of the following components:

i) a Notch ligand DSL domain;

ii) 2-4 and no more than 4 Notch ligand EGF domains;

iii) optionally all or part of a Notch ligand N-terminal domain; and

iv) optionally one or more heterologous amino acid sequences.

28. A conjugate as described in any one of paragraphs 1 to 25 comprising a modulator of Notch signalling consisting essentially of the following components:

i) a Notch ligand DSL domain;

ii) 2-3 and no more than 3 Notch ligand EGF domains;

iii) optionally all or part of a Notch ligand N-terminal domain; and

iv) optionally one or more heterologous amino acid sequences.

29. A conjugate as described in any one of paragraphs 1 to 25 comprising a modulator of Notch signalling consisting essentially of the following components:

i) a Notch ligand DSL domain;

ii) 3 Notch ligand EGF domains;

iii) optionally all or part of a Notch ligand N-terminal domain; and

iv) optionally one or more heterologous amino acid sequences.

30. A conjugate as described in any one of paragraphs 1 to 25 comprising a modulator of Notch signalling in the form of a protein or polypeptide comprising:

i) a Notch ligand DSL domain;

ii) 1-5 Notch ligand EGF domains;

iii) optionally all or part of a Notch ligand N-terminal domain; and

iv) optionally one or more heterologous amino acid sequences.

31. A conjugate as described in any one of paragraphs 1 to 25 comprising a modulator of Notch signalling in the form of a protein or polypeptide comprising:

i) a Notch ligand DSL domain;

ii) 2-4 Notch ligand EGF domains;

iii) optionally all or part of a Notch ligand N-terminal domain; and

iv) optionally one or more heterologous amino acid sequences.

32. A conjugate as described in any one of paragraphs 1 to 25 comprising a modulator of Notch signalling in the form of a protein or polypeptide comprising:

i) a Notch ligand DSL domain;

ii) 2-3 Notch ligand EGF domains;

iii) optionally all or part of a Notch ligand N-terminal domain; and

iv) optionally one or more heterologous amino acid sequences.

33. A conjugate as described in any one of paragraphs 1 to 25 comprising a modulator of Notch signalling in the form of a protein or polypeptide comprising:

i) a Notch ligand DSL domain;

ii) 3 Notch ligand EGF domains;

iii) optionally all or part of a Notch ligand N-terminal domain; and

iv) optionally one or more heterologous amino acid sequences.

34. A conjugate as described in any one of paragraphs 1 to 33 comprising Delta DSL or EGF domains.

35. A conjugate as described in any of paragraphs 1 to 34 comprising human Delta DSL or EGF domains.

36. A conjugate as described in any one of paragraphs 1 to 35 comprising a polypeptide which has at least 50% amino acid sequence similarity to the following sequence along the entire length of the latter:

MGSRCALALAVLSALLCQVWSSGVFELKLQEFVNKKGLLGNRNCCRGGAGPPPCACRTF
FRVCLKHYQASVSPEPPCTYGSAVTPVLGVDSFSLPDGGGADSAFSNPIRFPFGFTWPG
TFSLIIEALHTDSPDDLATENPERLISRLATQRHLTVGEEWSQDLHSSGRTDLKYSYRF
VCDEHYYGEGCSVFCRPRDDAFGHFTCGERGEKVCNPGWKGPYCTEPICLPGCDEQHGF
CDKPGECKCRVGWQGRYCDECIRYPGCLHGTCQQPWQCNCQEGWGGLFCNQDLNYCTHH
KPCKNGATCTNTGQGSYTCSCRPGYTGATCELGIDEC

37. A conjugate as described in paragraph 36 comprising a polypeptide which has at least 70% amino acid sequence similarity to the sequence of paragraph 33 along the entire length of the latter:

38. A conjugate as described in paragraph 36 comprising a polypeptide which has at least 90% amino acid sequence similarity to the sequence of paragraph 36 along the entire length of the latter:

39. A conjugate as described in any one of paragraphs 1 to 38 wherein at least one of the modulators of the Notch signalling pathway comprises an antibody.

40. A conjugate of the formula: POL(-R)ₙ

wherein POL is a polymeric support structure; each R represents a modulator of Notch signalling (each of which may be the same or different); and n is an integer being at least 2.

41. A conjugate of the formula: POL(-L-R)ₙ

wherein POL is a polymeric support structure; each R independently represents a modulator of Notch signalling (each of which may be the same or different); each L independently represents either an optional linker moiety (each of which may be the same or different) or a bond; and n is an integer being at least 2.

42. A conjugate as described in paragraph 40 or paragraph 41 wherein n is at least 5.

43. A conjugate as described in paragraph 42 wherein n is at least 20.

44. A conjugate as described in any one of paragraphs 40 to 43 wherein POL is a water soluble polymer.

45. A conjugate as described in any one of paragraphs 40 to 44 wherein POL is an optionally derivatised or activated polysaccharide polymer.

46. A conjugate as described in paragraph 45 wherein POL is an optionally derivatised or activated dextran polymer.

47. A conjugate as described in any one of paragraphs 40 to 45 wherein POL is an optionally derivatised or activated PEG polymer.

48. A conjugate as described in any one of paragraphs 40 to 47 wherein each L is a same or different protein or polypeptide comprising a Notch ligand DSL domain and at least 1 to 8 Notch ligand EGF-like domains.

49. A conjugate as described in any one of the preceding paragraphs for use as a medicament.

50. The use of a conjugate as described in any one of paragraphs 1 to 48 in the manufacture of a medicament for modulation of an immune response.

51. The use of a conjugate as described in any one of paragraphs 1 to 48 in the manufacture of a medicament for downregulation of an immune response.

52. A method of modulating an immune response in a mammal by administering a conjugate as described in any one of paragraphs 1 to 48.

53. A method for downregulating an immune response in a mammal by administering a conjugate as described in any one of paragraphs 1 to 48.

54. A method for preparing a conjugate as described in any one of paragraphs 1 to 48 by combining a plurality of modulators of the Notch signalling pathway with a polymeric support structure.

55. A method for preparing a conjugate as described in any one of paragraphs 1 to 48 by:

i) providing a polymeric support structure; and

ii) reacting the polymeric support structure with a plurality of modulators of Notch signalling.

56. A method for preparing a conjugate as described in any one of paragraphs 1 to 48 by:

i) providing a polymeric support structure;

ii) activating the polymeric support structure; and

iii) reacting the activated polymeric support structure with a plurality of modulators of Notch signalling.

57. A product comprising:

i) a conjugate as described in any one of paragraphs 1 to 48; and

ii) an antigen or antigenic determinant or a polynucleotide coding for an antigen or antigenic determinant;

as a combined preparation for simultaneous, contemporaneous, separate or sequential use for modulation of the immune system.

58. A product as described in paragraph 57 wherein the antigen or antigenic determinant is an autoantigen or antigenic determinant thereof or a polynucleotide coding for an autoantigen or antigenic determinant thereof.

59. A product as described in paragraph 57 wherein the antigen or antigenic determinant is an allergen or antigenic determinant thereof or a polynucleotide coding for an allergen or antigenic determinant thereof.

60. A product as described in paragraph 57 wherein the antigen or antigenic determinant is a transplant antigen or antigenic determinant thereof or a polynucleotide coding for a transplant antigen or antigenic determinant thereof.

61. A product as described in paragraph 57 wherein the antigen or antigenic determinant is a tumour antigen or antigenic determinant thereof or a polynucleotide coding for a tumour antigen or antigenic determinant thereof.

62. A product as described in paragraph 57 wherein the antigen or antigenic determinant is a pathogen antigen or antigenic determinant thereof or a polynucleotide coding for a pathogen antigen or antigenic determinant thereof.

63. A pathogen vaccine composition comprising:

i) a conjugate as described in any one of paragraphs 1 to 48; and

ii) a pathogen antigen or antigenic determinant thereof or a polynucleotide coding for a pathogen antigen or antigenic determinant thereof.

64. A cancer vaccine composition comprising:

i) a conjugate as described in any one of paragraphs 1 to 48; and

ii) a cancer antigen or antigenic determinant thereof or a polynucleotide coding for a cancer antigen or antigenic determinant thereof.

65. The use of a conjugate as described in any one of paragraphs 1 to 48 for the manufacture of a medicament for modulation of expression of a cytokine selected from IL-10, IL-5, IL-2, TNF-alpha, IFN-gamma or IL-13.

66. The use of a construct comprising a conjugate as described in any one of paragraphs 1 to 48 for the manufacture of a medicament for increase of IL-10 expression.

67. The use of a conjugate as described in any one of paragraphs 1 to 48 for the manufacture of a medicament for decrease of expression of a cytokine selected from IL-2, IL-5, TNF-alpha, IFN-gamma or IL-13.

68. The use of a conjugate as described in any one of paragraphs 1 to 48 for the manufacture of a medicament for generating an immune modulatory cytokine profile with increased IL-10 expression and reduced IL-5 expression.

69. The use of a conjugate as described in any one of paragraphs 1 to 48 for the manufacture of a medicament for generating an immune modulatory cytokine profile with increased IL-10 expression and reduced IL-2, IFN-gamma, IL-5, IL-13 and TNF-alpha expression.

70. A pharmaceutical composition comprising a conjugate as described in any one of paragraphs 1 to 48.

71. A pharmaceutical composition as described in paragraph 70 comprising a pharmaceutically acceptable carrier.

72. The use of a conjugate as described in any one of paragraphs 1 to 48 to modify cell differentiation in therapy.

73. The use of a conjugate as described in any one of paragraphs 1 to 48 to inhibit cell differentiation in therapy.

74. The use of a conjugate as described in any one of paragraphs 1 to 48 to promote cell differentiation in therapy.

75. The use of a conjugate as described in any one of paragraphs 1 to 48 to for the treatment of cancer.

### REFERENCES (INCORPORATED HEREIN BY REFERENCE)

Altman J D et al Science 1996 274: 94-6.
Artavanis-Tsakonas S, et al. (1995) Science 268:225-232.
Artavanis-Tsakonas S, et al. (1999) Science 284:770-776.
Brucker K, et al. (2000) Nature 406:411-415.
Camilli et al. (1994) Proc Natl Acad Sci USA 91:2634-2638.
Chee M. et al. (1996) Science 274:601-614.
Hemmati-Brivanlou and Melton (1997) Cell 88:13-17.
Hicks C, et al. (2000) Nat. Cell. Biol. 2:515-520.
Iemura et al. (1998) PNAS 95:9337-9345.
Irvine K D (1999) Curr. Opin. Genet. Devel. 9:434-441.
Ju B J, et al. (2000) Nature 405:191-195.
Leimeister C. et al. (1999) Mech Dev 85(1-2):173-7.
Li et al. (1998) Immunity 8(1):43-55.
Lieber, T. et al. (1993) Genes Dev 7(10):1949-65.
Lu, F. M. et al. (1996) Proc Natl Acad Sci 93(11):5663-7.
Matsuno K, et al. (1998) Nat. Genet. 19:74-78.
Matsuno, K. et al. (1995) Development 121(8):2633-44.
Medhzhitov et al. (1997) Nature 388:394-397.
Meuer S. et al (2000) Rapid Cycle Real-time PCR, Springer-Verlag Berlin and Heidelberg GmbH & Co.
Moloney D J, et al. (2000) Nature 406:369-375.
Munro S, Freeman M. (2000) Curr. Biol. 10:813-820.
Ordentlich et al. (1998) Mol. Cell. Biol. 18:2230-2239.
Osborne B, Miele L. (1999) Immunity 11:653-663.
Panin V M, et al. (1997) Nature 387:908-912.
Sasai et al. (1994) Cell 79:779-790.
Schroeter E H, et al. (1998) Nature 393:382-386.
Schroeter, E. H. et al. (1998) Nature 393(6683):382-6.
Struhl G, Adachi A. (1998) Cell 93:649-660.
Struhl, G. et al. (1998) Cell 93(4):649-60.
Takebayashi K. et al. (1994) J Biol Chem 269(7):150-6.
Tamura K, et al. (1995) Curr. Biol. 5:1416-1423.
Valenzuela et al. (1995) J. Neurosci. 15:6077-6084.
Weinmaster G. (2000) Curr. Opin. Genet. Dev. 10:363-369.
Wilson and Hemmati-Brivanlou (1997) Neuron 18:699-710.
Zhao et al. (1995) J. Immunol. 155:3904-3911.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry, biology or related fields are intended to be within the scope of the following claims.

1. A conjugate comprising a plurality of modulators of the Notch signalling pathway chemically bound to a support structure, wherein the modulators are the same as or different from one another.

2. The conjugate as claimed in claim 1, wherein the support structure is a molecular support structure.

3. The conjugate as claimed in claim 1, wherein the modulators of the Notch signalling pathway are chemically cross-linked.

4. The conjugate as claimed in claim 1, wherein the support structure has a molecular weight of between about 500 and about 100,000 Da.

5. The conjugate as claimed in claim 1, wherein the support structure comprises a polymeric material.

6. The conjugate as claimed in claim 5, wherein the polymeric material is water soluble.

7. The conjugate as claimed in claim 5, wherein the polymeric material comprises an optionally derivatised or activated polysaccharide polymer, an optionally derivatised or activated dextran polymer, an optionally derivatised or activated polyethylene glycol, or a residue thereof.

8. The conjugate as claimed in claim 1, wherein at least one of the modulators of the Notch signalling pathway is coupled to the support structure via a linker moiety.

9. The conjugate as claimed in claim 8, wherein each of the modulators of the Notch signalling pathway is coupled to the support structure via a separate linker moiety, wherein the linker moieties are the same as or different from one another.

10. The conjugate as claimed in claim 8 comprising at least five modulators of the Notch signalling pathway.

11. The conjugate as claimed in claim 8, wherein the linker comprises an acid, basic, aldehyde, ether or ester reactive group or a residue thereof.

12. The conjugate as claimed in claim 8, wherein the linker moiety is a succinimidyl propionate, succinimidyl butanoate, N-hydroxysuccinimide, benzotriazole carbonate, propionaldehyde, maleimide or forked maleimide, biotin, vinyl derivative or phospholipid.

13. The conjugate as claimed in claim 8, wherein the linker is a protein or polypeptide comprising a Notch ligand DSL domain and at least 1 to 8 Notch ligand EGF-like domains.

14. The conjugate as claimed in claim 1 comprising at least three modulators of the Notch signalling pathway.

15. The conjugate as claimed in claim 1, wherein at least one of the modulators of the Notch signalling pathway is an agent capable of activating a Notch receptor.

16. The conjugate as claimed in claim 1, wherein at least one of the modulators of the Notch signalling pathway comprises a Notch ligand or a fragment, derivative, homologue, analogue or allelic variant thereof.

17. The conjugate as claimed in claim 1, wherein at least one of the modulators of the Notch signalling pathway comprises a Delta protein; a Serrate/Jagged protein; a DSL domain; an EGF-like domain; a fragment, derivative, homologue, analogue or allelic variant thereof; or a fusion protein comprising a segment of a Notch ligand extracellular domain and an immunoglobulin Fc segment.

18. The conjugate as claimed in claim 17, wherein at least one of the modulators of the Notch signalling pathway comprises a protein or polypeptide comprising at least one Notch ligand DSL domain and at least 1 Notch ligand EGF domain.

19. The conjugate as claimed in claim 17, wherein at least one of the modulators of the Notch signalling pathway comprises a protein or polypeptide comprising at least one Notch ligand DSL domain and at least 2 Notch ligand EGF domains.

20. The conjugate as claimed in claim 17 comprising a Delta DSL or a Delta EGF domain.

21. The conjugate as claimed in claim 1 comprising a modulator of Notch signalling, wherein the modulator of Notch signalling consists essentially of:
i) a Notch ligand DSL domain;
ii) 1-5 and no more than 5 Notch ligand EGF domains;
iii) optionally, all or part of a Notch ligand N-terminal domain; and
iv) optionally, one or more heterologous amino acid sequences.

22. The conjugate as claimed in claim 1 comprising a modulator of Notch signalling, wherein the modulator of Notch signalling consists essentially of:
i) a Notch ligand DSL domain;
ii) 2-4 and no more than 4 Notch ligand EGF domains;
iii) optionally, all or part of a Notch ligand N-terminal domain; and
iv) optionally, one or more heterologous amino acid sequences.

23. The conjugate as claimed in claim 1 comprising a modulator of Notch signalling, wherein the modulator of Notch signalling consists essentially of:
i) a Notch ligand DSL domain;
ii) 2-3 and no more than 3 Notch ligand EGF domains;
iii) optionally, all or part of a Notch ligand N-terminal domain; and
iv) optionally, one or more heterologous amino acid sequences.

24. The conjugate as claimed in claim 1 comprising a modulator of Notch signalling, wherein the modulator of Notch signalling consists essentially of:
i) a Notch ligand DSL domain;
ii) 3 Notch ligand EGF domains;
iii) optionally, all or part of a Notch ligand N-terminal domain; and
iv) optionally, one or more heterologous amino acid sequences.

25. The conjugate as claimed in claim 1 comprising a polypeptide which has at least about 50% amino acid sequence similarity to the following sequence along the entire length of the latter:

| | |
|---|---|
| MGSRCALALAVLSALLCQVWSSGVFELKLQEFVNKKGLLGNRNCCRGGAGPPPCACRTF | (SEQ ID NO: 41) |
| FRVCLKHYQASVSPEPPCTYGSAVTPVLGVDSFSLPDCGGADSAFSNPIRFPFGFTWPG | |
| TFSLITEALHTDSPDDLATENPERLISRLATQRHLTVGEEWSQDLHSSGRTDLKYSYRF | |
| VCDEHYYGEGCSVFCRPRDDAFGHFTCGERGEKVCNPCWKGPYCTEPTCLPGCDEQHGF | |
| CDKPGECKCRVGWQGRYCDECIRYPGCLHGTCQQPWQCNCQECWGGLFCNQDLNYCTHH | |
| KPCKNGATCTNTGQGSYTCSCRPGYTGATCELGIDEC | |

26. The conjugate as claimed in claim 25 comprising a polypeptide which has at least about 70% amino acid sequence similarity to the sequence of claim 25 along the entire length of the latter.

27. The conjugate as claimed in claim 25 comprising a polypeptide which has at least about 90% amino acid sequence similarity to the sequence of claim 25 along the entire length of the latter.

28. The conjugate as claimed in claim 1, wherein at least one of the modulators of the Notch signalling pathway comprises an antibody.

29. A composition comprising the conjugate as claimed in claim 1 and a pharmaceutically acceptable carrier.


SEQUENCE LISTING (69 sequences)

SEQ ID NO: 1 - protein, 43 aa, Artificial Sequence
  Description of Artificial Sequence Synthetic consensus sequence
CXXXXXXXXCXXXCXXXXXXXXXXXCXXXXXXXCXXXXXXXXC

SEQ ID NO: 2 - protein, 43 aa, Artificial Sequence; identical to SEQ ID NO: 1
  Description of Artificial Sequence Synthetic consensus sequence

SEQ ID NO: 3 - protein, 43 aa, Artificial Sequence
  Description of Artificial Sequence Synthetic consensus sequence
CXXXYYXXXCXXXCRPRXDXFGHXXCXXXGXXXCXXGWXGXXC

SEQ ID NO: 4 - DNA, 66 bp, Artificial Sequence
  Description of Artificial Sequence Synthetic oligonucleotide
agcttgcggccgcgggcccagcggtggtggacctcactgagaagctagaggcttccaccaaaggcc

SEQ ID NO: 5 - DNA, 58 bp, Artificial Sequence
  Description of Artificial Sequence Synthetic oligonucleotide
tttggtggaagcctctagcttctcagtgaggtccaccaccgctgggcccgcggccgca

SEQ ID NO: 6 - DNA, 50 bp, Artificial Sequence
  Description of Artificial Sequence Synthetic oligonucleotide
agcttgccgccaccatgggcagtcggtgcgcgctggccctggcggtgctc

SEQ ID NO: 7 - DNA, 40 bp, Artificial Sequence
  Description of Artificial Sequence Synthetic oligonucleotide
cgccagggccagcgcgcaccgactgcccatggtggcggca

SEQ ID NO: 8 - DNA, 38 bp, Artificial Sequence
  Description of Artificial Sequence Synthetic oligonucleotide
tcggccttgctgtgtcaggtctggagctctggggtgtt

SEQ ID NO: 9 - DNA, 46 bp, Artificial Sequence
  Description of Artificial Sequence Synthetic oligonucleotide
cgaacaccccagagctccagacctgacacagcaaggccgagagcac

SEQ ID NO: 10 - protein, 864 aa, Homo sapiens
MGSRCALALAVLSALLCQVWSSGVFELKLQEFVNKKGLLGNRNCCRGGAGPPPCACRTFFRVCLKHYQASVSPEPPCTYGSAVTPVLGVDSFSLPDGGGADSAFSNPIRFPFGFTWPGTFSLIIEALHTDSPDDLATENPERLISRLATQRHLTVGEEWSQDLHSSGRTDLKYSYRFVCDEHYYGEGCSVFCRPRDDAFGHFTCGERGEKVCNPGWKGPYCTEPICLPGCDEQHGFCDKPGECKCRVGWQGRYCDECIRYPGCLHGTCQQPWQCNCQEGWGGLFCNQDLNYCTHHKPCKNGATCTNTGQGSYTCSCRPGYTGATCELGIDECDPSPCKNGGSCTDLENSYSCTCPPGFYGKICELSAMTCADGPCFNGGRCSDSPDGGYSCRCPVGYSGFNCEKKIDYCSSSPCSNGAKCVDLGDAYLCRCQAGFSGRHCDDNVDDCASSPCANGGTCRDGVNDFSCTCPPGYTGRNCSAPVSRCEHAPCHNGATCHERGHGYVCECARGYGGPNCQFLLPELPPGPAVVDLTEKLEASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - DNA, 20 bp, Artificial Sequence
  Description of Artificial Sequence Synthetic oligonucleotide
gtgctgttacccgtacggta

SEQ ID NO: 12 - DNA, 20 bp, Artificial Sequence
  Description of Artificial Sequence Synthetic oligonucleotide
cgtacgggtaacagcacaag

SEQ ID NO: 13 - DNA, 40 bp, Artificial Sequence
  Description of Artificial Sequence Synthetic oligonucleotide
agcttgccgccaccatgggttccccacggacacgcggccg

SEQ ID NO: 14 - DNA, 39 bp, Artificial Sequence
  Description of Artificial Sequence Synthetic oligonucleotide
gaccggccgcgtgtccgtggggaacccatggtggcggca

SEQ ID NO: 15 - DNA, 27 bp, Artificial Sequence
  Description of Artificial Sequence Synthetic oligonucleotide
gtccgcaccttgtgggtacccgtacgg

SEQ ID NO: 16 - DNA, 28 bp, Artificial Sequence
  Description of Artificial Sequence Synthetic oligonucleotide
aattccgtacgggtacccacaaggtgcg

SEQ ID NO: 17 - DNA, 22 bp, Artificial Sequence
  Description of Artificial Sequence Synthetic oligonucleotide
gatctcgcttccaccaagggcc

SEQ ID NO: 18 - DNA, 14 bp, Artificial Sequence
  Description of Artificial Sequence Synthetic oligonucleotide
cttggtggaagcga

SEQ ID NO: 19 - protein, 626 aa, Homo sapiens [sequence omitted]

SEQ ID NO: 20 - DNA, 26 bp, Artificial Sequence
  Description of Artificial Sequence Synthetic primer
caccatgggcagtcggtgcgcgctgg

SEQ ID NO: 21 - DNA, 22 bp, Artificial Sequence
  Description of Artificial Sequence Synthetic primer
gtagttcaggtcctggttgcag

SEQ ID NO: 22 - DNA, 26 bp, Artificial Sequence
  Description of Artificial Sequence Synthetic primer
caccatgggcagtcggtgcgcgctgg

SEQ ID NO: 23 - DNA, 45 bp, Artificial Sequence
  Description of Artificial Sequence Synthetic primer
ggatatgggcccttggtggaagcgtagttcaggtcctggttgcag

SEQ ID NO: 24 - protein, 291 aa, Homo sapiens
MGSRCALALAVLSALLCQVWSSGVFELKLQEFVNKKGLLGNRNCCRGGAGPPPCACRTFFRVCLKHYQASVSPEPPCTYGSAVTPVLGVDSFSLPDGGGADSAFSNPIRFPFGFTWPGTFSLIIEALHTDSPDDLATENPERLISRLATQRHLTVGEEWSQDLHSSGRTDLKYSYRFVCDEHYYGEGCSVFCRPRDDAFGHFTCGERGEKVCNPGWKGPYCTEPICLPGCDEQHGFCDKPGECKCRVGWQGRYCDECIRYPGCLHGTCQQPWQCNCQEGWGGLFCNQDLNY

SEQ ID NO: 25 - DNA, 26 bp, Artificial Sequence
  Description of Artificial Sequence Synthetic primer
caccatgggcagtcggtgcgcgctgg

SEQ ID NO: 26 - DNA, 45 bp, Artificial Sequence
  Description of Artificial Sequence Synthetic primer
ggatatgggcccttggtggaagcctcgtcaatccccagctcgcag

SEQ ID NO: 27 - protein, 331 aa, Homo sapiens
MGSRCALALAVLSALLCQVWSSGVFELKLQEFVNKKGLLGNRNCCRGGAGPPPCACRTFFRVCLKHYQASVSPEPPCTYGSAVTPVLGVDSFSLPDGGGADSAFSNPIRFPFGFTWPGTFSLIIEALHTDSPDDLATENPERLISRLATQRHLTVGEEWSQDLHSSGRTDLKYSYRFVCDEHYYGEGCSVFCRPRDDAFGHFTCGERGEKVCNPGWKGPYCTEPICLPGCDEQHGFCDKPGECKCRVGWQGRYCDECIRYPGCLHGTCQQPWQCNCQEGWGGLFCNQDLNYCTHHKPCKNGATCTNTGQGSYTCSCRPGYTGATCELGIDE

SEQ ID NO: 28 - DNA, 26 bp, Artificial Sequence
  Description of Artificial Sequence Synthetic primer
caccatgggcagtcggtgcgcgctgg

SEQ ID NO: 29 - DNA, 22 bp, Artificial Sequence
  Description of Artificial Sequence Synthetic primer
ggtcatggcactcaattcacag

SEQ ID NO: 30 - DNA, 26 bp, Artificial Sequence
  Description of Artificial Sequence Synthetic primer
caccatgggcagtcggtgcgcgctgg

SEQ ID NO: 31 - DNA, 45 bp, Artificial Sequence
  Description of Artificial Sequence Synthetic primer
ggatatgggcccttggtggaagcggtcatggcactcaattcacag

SEQ ID NO: 32 - protein, 369 aa, Homo sapiens
MGSRCALALAVLSALLCQVWSSGVFELKLQEFVNKKGLLGNRNCCRGGAGPPPCACRTFFRVCLKHYQASVSPEPPCTYGSAVTPVLGVDSFSLPDGGGADSAFSNPIRFPFGFTWPGTFSLIIEALHTDSPDDLATENPERLISRLATQRHLTVGEEWSQDLHSSGRTDLKYSYRFVCDEHYYGEGCSVFCRPRDDAFGHFTCGERGEKVCNPGWKGPYCTEPICLPGCDEQHGFCDKPGECKCRVGWQGRYCDECIRYPGCLHGTCQQPWQCNCQEGWGGLFCNQDLNYCTHHKPCKNGATCTNTGQGSYTCSCRPGYTGATCELGIDECDPSPCKNGGSCTDLENSYSCTCPPGFYGKICELSAMT

SEQ ID NO: 33 - DNA, 26 bp, Artificial Sequence
  Description of Artificial Sequence Synthetic primer
caccatgggcagtcggtgcgcgctgg

SEQ ID NO: 34 - DNA, 25 bp, Artificial Sequence
  Description of Artificial Sequence Synthetic primer
cctgctgacgggggcactgcagttc

SEQ ID NO: 35 - DNA, 26 bp, Artificial Sequence
  Description of Artificial Sequence Synthetic primer
caccatgggcagtcggtgcgcgctgg

SEQ ID NO: 36 - DNA, 48 bp, Artificial Sequence
  Description of Artificial Sequence Synthetic primer
ggatatgggcccttggtggaagccctgctgacgggggcactgcagttc

SEQ ID NO: 37 - protein, 484 aa, Homo sapiens
MGSRCALALAVLSALLCQVWSSGVFELKLQEFVNKKGLLGNRNCCRGGAGPPPCACRTFFRVCLKHYQASVSPEPPCTYGSAVTPVLGVDSFSLPDGGGADSAFSNPIRFPFGFTWPGTFSLIIEALHTDSPDDLATENPERLISRLATQRHLTVGEEWSQDLHSSGRTDLKYSYRFVCDEHYYGEGCSVFCRPRDDAFGHFTCGERGEKVCNPGWKGPYCTEPICLPGCDEQHGFCDKPGECKCRVGWQGRYCDECIRYPGCLHGTCQQPWQCNCQEGWGGLFCNQDLNYCTHHKPCKNGATCTNTGQGSYTCSCRPGYTGATCELGIDECDPSPCKNGGSCTDLENSYSCTCPPGFYGKICELSAMTCADGPCFNGGRCSDSPDGGYSCRCPVGYSGFNCEKKIDYCSSSPCSNGAKCVDLGDAYLCRCQAGFSGRHCDDNVDDCASSPCANGGTCRDGVNDFSCTCPPGYTGRNCSAPVSR

SEQ ID NO: 38 - DNA, 26 bp, Artificial Sequence
  Description of Artificial Sequence Synthetic primer
caccatgggcagtcggtgcgcgctgg

SEQ ID NO: 39 - DNA, 45 bp, Artificial Sequence
  Description of Artificial Sequence Synthetic primer
gtctacgtttaaacttaacactcgtcaatccccagctcgcaggtg

SEQ ID NO: 40 - DNA, 999 bp, Homo sapiens
atgggcagtcggtgcgcgctggccctggcggtgctctcggccttgctgtgtcaggtctggagctctggggtgttcgaactgaagctgcaggagttcgtcaacaagaaggggctgctggggaaccgcaactgctgccgcgggggcgcggggccaccgccgtgcgcctgccggaccttcttccgcgtgtgcctcaagcactaccaggccagcgtgtcccccgagccgccctgcacctacggcagcgccgtcacccccgtgctgggcgtcgactccttcagtctgcccgacggcgggggcgccgactccgcgttcagcaaccccatccgcttccccttcggcttcacctggccgggcaccttctctctgattattgaagctctccacacagattctcctgatgacctcgcaacagaaaacccagaaagactcatcagccgcctggccacccagaggcacctgacggtgggcgaggagtggtcccaggacctgcacagcagcggccgcacggacctcaagtactcctaccgcttcgtgtgtgacgaacactactacggagagggctgctccgttttctgccgtccccgggacgatgccttcggccacttcacctgtggggagcgtggggagaaagtgtgcaaccctggctggaaagggccctactgcacagagccgatctgcctgcctggatgtgatgagcagcatggattttgtgacaaaccaggggaatgcaagtgcagagtgggctggcagggccggtactgtgacgagtgtatccgctatccaggctgtctccatggcacctgccagcagccctggcagtgcaactgccaggaaggctgggggggccttttctgcaaccaggacctgaactactgcacacaccataagccctgcaagaatggagccacctgcaccaacacgggccaggggagctacacttgctcttgccggcctgggtacacaggtgccacctgcgagctggggattgacgagtgttaa

SEQ ID NO: 41 - protein, 332 aa, Homo sapiens
MGSRCALALAVLSALLCQVWSSGVFELKLQEFVNKKGLLGNRNCCRGGAGPPPCACRTFFRVCLKHYQASVSPEPPCTYGSAVTPVLGVDSFSLPDGGGADSAFSNPIRFPFGFTWPGTFSLIIEALHTDSPDDLATENPERLISRLATQRHLTVGEEWSQDLHSSGRTDLKYSYRFVCDEHYYGEGCSVFCRPRDDAFGHFTCGERGEKVCNPGWKGPYCTEPICLPGCDEQHGFCDKPGECKCRVGWQGRYCDECIRYPGCLHGTCQQPWQCNCQEGWGGLFCNQDLNYCTHHKPCKNGATCTNTGQGSYTCSCRPGYTGATCELGIDEC

SEQ ID NO: 42 - DNA, 26 bp, Artificial Sequence
  Description of Artificial Sequence Synthetic oligonucleotide
gatctggggggctataaaagggggta

SEQ ID NO: 43 - DNA, 26 bp, Artificial Sequence
  Description of Artificial Sequence Synthetic oligonucleotide
agcttacccccttttatagcccccca

SEQ ID NO: 44 - DNA, 50 bp, Artificial Sequence
  Description of Artificial Sequence Synthetic oligonucleotide
gatcccgactcgtgggaaaatgggcggaagggcaccgtgggaaaatagta

SEQ ID NO: 45 - DNA, 50 bp, Artificial Sequence
  Description of Artificial Sequence Synthetic oligonucleotide
gatctactattttcccacggtgcccttccgcccattttcccacgagtcgg

SEQ ID NO: 46 - protein, 63 aa, Drosophila melanogaster
WKTNKSESQYTSLEYDFRVTCDLNYYGSGCAKFCRPRDDSFGHSTCSETGEIICLTGWQGDYC

SEQ ID NO: 47 - protein, 63 aa, Homo sapiens
WSQDLHSSGRTDLKYSYRFVCDEHYYGEGCSVFCRPRDDAFGHFTCGERGEKVCNPGWKGPYC

SEQ ID NO: 48 - protein, 63 aa, Mus musculus
WSQDLHSSGRTDLRYSYRFVCDEHYYGEGCSVFCRPRDDAFGHFTCGDRGEKMCDPGWKGQYC

SEQ ID NO: 49 - protein, 63 aa, Rattus norvegicus
WSQDLHSSGRTDLRYSYRFVCDEHYYGEGCSVFCRPRDDAFGHFTCGERGEKMCDPGWKGQYC

SEQ ID NO: 50 - protein, 63 aa, Mus musculus
WRTDEQNDTLTRLSYSYRVICSDNYYGESCSRLCKKRDDHFGHYECQPDGSLSCLPGWTGKYC

SEQ ID NO: 51 - protein, 63 aa, Homo sapiens
WLLDEQTSTLTRLRYSYRVICSDNYYGDNCSRLCKKRNDHFGHYVCQPDGNLSCLPGWTGEYC

SEQ ID NO: 52 - protein, 63 aa, Rattus norvegicus
WQTLKQNTGIAHFEYQIRVTCDDHYYGFGCNKFCRPRDDFFGHYACDQNGNKTCMEGWMGPEC

SEQ ID NO: 53 - protein, 63 aa, Mus musculus
WQTLKQNTGIAHFEYQIRVTCDDHYYGFGCNKFCRPRDDFFGHYACDQNGNKTCMEGWMGPDC

SEQ ID NO: 54 - protein, 63 aa, Homo sapiens
WQTLKQNTGVAHFEYQIRVTCDDYYYGFGCNKFCRPRDDFFGHYACDQNGNKTCMEGWMGREC

SEQ ID NO: 55 - protein, 63 aa, Gallus gallus
WQTLKHNTGAAHFEYQIRVTCAEHYYGFGCNKFCRPRDDFFTHHTCDQNGNKTCLEGWTGPEC

SEQ ID NO: 56 - protein, 63 aa, Gallus gallus
WKTLQFNGPVANFEVQIRVKCDENYYSALCNKFCGPRDDFVGHYTCDQNGNKACMEGWMGEEC

SEQ ID NO: 57 - protein, 63 aa, Mus musculus
WKSLHFSGHVAHLELQIRVRCDENYYSATCNKFCRPRNDFFGHYTCDQYGNKACMDGWMGKEC

SEQ ID NO: 58 - protein, 63 aa, Homo sapiens; identical to SEQ ID NO: 57

SEQ ID NO: 59 - protein, 63 aa, Rattus norvegicus; identical to SEQ ID NO: 57

SEQ ID NO: 60 - protein, 63 aa, Homo sapiens; identical to SEQ ID NO: 57

SEQ ID NO: 61 - protein, 63 aa, Drosophila melanogaster
WKTLDHIGRNARITYRVRVQCAVTYYNTTCTTFCRPRDDQFGHYACGSEGQKLCLNGWQGVNC

SEQ ID NO: 62 - protein, 723 aa, Homo sapiens [sequence omitted]

SEQ ID NO: 63 - protein, 618 aa, Homo sapiens
MVSPRMSGLLSQTVILALIFLPQTRPAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYLLPPALGLLVAAGVAGAALLLVHVRRRGHSQDAGSRLLAGTPEPSVHALPDALNNLRTQEGSGDGPSSSVDWNRPEDVDPQGIYVISAPSIYAREVATPLFPPLHTGRAGQRQHLLFPYPSSILSVK

SEQ ID NO: 64 - protein, 685 aa, Homo sapiens
MAAASRSASGWALLLLVALWQQRAAGSGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKVDRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVGLPPSFPWVAVSLGVGLAVLLVLLGMVAVAVRQLRLRRPDDGSREAMNNLSDFQKDNLIPAAQLKNTNQKKELEVDCGLDKSNCGKQQNHTLDYNLAPGPLGRGTMPGKFPHSDKSLGEKAPLRLHSEKPECRISAICSPRDSMYQSVCLISEERNECVIATEV

SEQ ID NO: 65 - protein, 1218 aa, Homo sapiens
MRSPRTRGRSGRPLSLLLALLCALRAKVCGASGQFELEILSMQNVNGELQNGNCCGGARNPGDRKCTRDECDTYFKVCLKEYQSRVTAGGPCSFGSGSTPVIGGNTFNLKASRGNDRNRIVLPFSFAWPRSYTLLVEAWDSSNDTVQPDSIIEKASHSGMINPSRQWQTLKQNTGVAHFEYQIRVTCDDYYYGFGCNKFCRPRDDFFGHYACDQNGNKTCMEGWMGPECNRAICRQGCSPKHGSCKLPGDCRCQYGWQGLYCDKCIPHPGCVHGICNEPWQCLCETNWGGQLCDKDLNYCGTHQPCLNGGTCSNTGPDKYQCSCPEGYSGPNCEIAEHACLSDPCHNRGSCKETSLGFECECSPGWTGPTCSTNIDDCSPNNCSHGGTCQDLVNGFKCVCPPQWTGKTCQLDANECEAKPCVNAKSCKNLIASYYCDCLPGWMGQNCDININDCLGQCQNDASCRDLVNGYRCICPPGYAGDHCERDIDECASNPCLNGGHCQNEINRFQCLCPTGFSGNLCQLDIDYCEPNPCQNGAQCYNRASDYFCKCPEDYEGKNCSHLKDHCRTTPCEVIDSCTVAMASNDTPEGVRYISSNVCGPHGKCKSQSGGKFTCDCNKGFTGTYCHENINDCESNPCRNGGTCIDGVNSYKCICSDGWEGAYCETNINDCSQNPCHNGGTCRDLVNDFYCDCKNGWKGKTCHSRDSQCDEATCNNGGTCYDEGDAFKCMCPGGWEGTTCNIARNSSCLPNPCHNGGTCVVNGESFTCVCKEGWEGPICAQNTNDCSPHPCYNSGTCVDGDNWYRCECAPGFAGPDCRININECQSSPCAFGATCVDEINGYRCVCPPGHSGAKCQEVSGRPCITMGSVIPDGAKWDDDCNTCQCLNGRIACSKVWCGPRPCLLHKGHSECPSGQSCIPILDDQCFVHPCTGVGECRSSSLQPVKTKCTSDSYYQDNCANITFTFNKEMMSPGLTTEHICSELRNLNILKNVSAEYSIYIACEPSPSANNEIHVAISAEDIRDDGNPIKEITDKIIDLVSKRDGNSSLIAAVAEVRVQRRPLKNRTDFLVPLLSSVLTVAWICCLVTAFYWCLRKRRKPGSHTHSASEDNTTNNVREQLNQIKNPIEKHGANTVPIKDYENKNSKMSKIRTHNSEVEEDDMDKHQQKARFAKQPAYTLVDREEKPPNGTPTKHPNWTNKQDNRDLESAQSLNRMEYIV

SEQ ID NO: 66 - protein, 1238 aa, Homo sapiens [sequence omitted]

SEQ ID NO: 67 - protein, 2556 aa, Homo sapiens
  MOD_RES (891): Variable amino acid
MPPLLAPLLCLALLPALAARGPRCSQPGETCLNGGKCEAANGTEACVCGGAFVGPRCQDPNPCLSTPCKNAGTCHVVDRRGVADYACSCALGFSGPLCLTPLDNACLTNPCRNGGTCDLLTLTEYKCRCPPGWSGKSCQQADPCASNPCANGGQCLPFEASYICHCPPSFHGPTCRQDVNECGQKPRLCRHGGTCHNEVGSYRCVCRATHTGPNCERPYVPCSPSPCQNGGTCRPTGDVTHECACLPGFTGQNCEENIDDCPGNNCKNGGACVDGVNTYNCPCPPEWTGQYCTEDVDECQLMPNACQNGGTCHNTHGGYNCVCVNGWTGEDCSENIDDCASAACFHGATCHDRVASFYCECPHGRTGLLCHLNDACISNPCNEGSNCDTNPVNGKAICTCPSGYTGPACSQDVDECSLGANPCEHAGKCINTLGSFECQCLQGYTGPRCEIDVNECVSNPCQNDATCLDQIGEFQCMCMPGYEGVHCEVNTDECASSPCLHNGRCLDKINEFQCECPTGFTGHLCQYDVDECASTPCKNGAKCLDGPNTYTCVCTEGYTGTHCEVDIDECDPDPCHYGSCKDGVATFTCLCRPGYTGHHCETNINECSSQPCRLRGTCQDPDNAYLCFCLKGTTGPNCEINLDDCASSPCDSGTCLDKIDGYECACEPGYTGSMCNSNIDECAGNPCHNGGTCEDGINGFTCRCPEGYHDPTCLSEVNECNSNPCVHGACRDSLNGYKCDCDPGWSGTNCDINNNECESNPCVNGGTCKDMTSGIVCTCREGFSGPNCQTNINECASNPCLNKGTCIDDVAGYKCNCLLPYTGATCEVVLAPCAPSPCRNGGECRQSEDYESFSCVCPTAGAKGQTCEVDINECVLSPCRHGASCQNTHGXYRCHCQAGYSGRNCETDIDDCRPNPCHNGGSCTDGINTAFCDCLPGFRGTFCEEDINECASDPCRNGANCTDCVDSYTCTCPAGFSGIHCENNTPDCTESSCFNGGTCVDGINSFTCLCPPGFTGSYCQHVVNECDSRPCLLGGTCQDGRGLHRCTCPQGYTGPNCQNLVHWCDSSPCKNGGKCWQTHTQYRCECPSGWTGLYCDVPSVSCEVAAQRQGVDVARLCQHGGLCVDAGNTHHCRCQAGYTGSYCEDLVDECSPSPCQNGATCTDYLGGYSCKCVAGYHGVNCSEEIDECLSHPCQNGGTCLDLPNTYKCSCPRGTQGVHCEINVDDCNPPVDPVSRSPKCFNNGTCVDQVGGYSCTCPPGFVGERCEGDVNECLSNPCDARGTQNCVQRVNDFHCECRAGHTGRRCESVINGCKGKPCKNGGTCAVASNTARGFICKCPAGFEGATCENDARTCGSLRCLNGGTCISGPRSPTCLCLGPFTGPECQFPASSPCLGGNPCYNQGTCEPTSESPFYRCLCPAKFNGLLCHILDYSFGGGAGRDIPPPLIEEACELPECQEDAGNKVCSLQCNNHACGWDGGDCSLNFNDPWKNCTQSLQCWKYFSDGHCDSQCNSAGCLFDGFDCQRAEGQCNPLYDQYCKDHFSDGHCDQGCNSAECEWDGLDCAEHVPERLAAGTLVVVVLMPPEQLRNSSFHFLRELSRVLHTNVVFKRDAHGQQMIFPYYGREEELRKHPIKRAAEGWAAPDALLGQVKASLLPGGSEGGRRRRELDPMDVRGSIVYLEIDNRQCVQASSQCFQSATDVAAFLGALASLGSLNIPYKIEAVQSETVEPPPPAQLHFMYVAAAAFVLLFFVGCGVLLSRKRRRQHGQLWFPEGFKVSEASKKKRREPLGEDSVGLKPLKNASDGALMDDNQNEWGDEDLETKKFRFEEPVVLPDLDDQTDHRQWTQQHLDAADLRMSAMAPTPPQGEVDADCMDVNVRGPDGFTPLMIASCSGGGLETGNSEEEEDAPAVISDFIYQGASLHNQTDRTGETALHLAARYSRSDAAKRLLEASADANIQDNMGRTPLHAAVSADAQGVFQILIRNRATDLDARMHDGTTPLILAARLAVEGMLEDLINSHADVNAVDDLGKSALHWAAAVNNVDAAVVLLKNGANKDMQNNREETPLFLAAREGSYETAKVLLDHFANRDITDHMDRLPRDIAQERMHHDIVRLLDEYNLVRSPQLHGAPLGGTPTLSPPLCSPNGYLGSLKPGVQGKKVRKPSSKGLACGSKEAKDLKARRKKSQDGKGCLLDSSGMLSPVDSLESPHGYLSDVASPPLLPSPFQQSPSVPLNHLPGMPDTHLGIGHLNVAAKPEMAALGGGGRLAFETGPPRLSHLPVASGTSTVLGSSSGGALNFTVGGSTSLNGQCEWLSRLQSGMVPNQYNPLRGSVAPGPLSTQAPSLQHGMVGPLHSSLAASALSQMMSYQGLPSTRLATQPHLVQTQQVQPQNLQMQQQNLQPANIQQQQSLQPPPPPPQPHLGVSSAASGHLGRSFLSGEPSQADVQPLGPSSLAVHTILPQESPALPTSLPSSLVPPVTAAQFLTPPSQHSYSSPVDNTPSHQLQVPEHPFLTPSPESPDQWSSSSPHSNVSDWSEGVSSPPTSMQSQIARIPEAFK

SEQ ID NO: 68 - protein, 2471 aa, Homo sapiens
MPALRPALLWALLALWLCCAAPAHALQCRDGYEPCVNEGMCVTYHNGTGYCKCPEGFLGEYCQHRDPCEKNRCQNGGTCVAQAMLGKATCRCASGFTGEDCQYSTSHPCFVSRPCLNGGTCHMLSRDTYECTCQVGFTGKECQWTDACLSHPCANGSTCTTVANQFSCKCLTGFTGQKCETDVNECDIPGHCQHGGTCLNLPGSYQCQCPQGFTGQYCDSLYVPCAPSPCVNGGTCRQTGDFTFECNCLPGFEGSTCERNIDDCPNHRCQNGGVCVDGVNTYNCRCPPQWTGQFCTEDVDECLLQPNACQNGGTCANRNGGYGCVCVNGWSGDDCSENIDDCAFASCTPGSTCIDRVASFSCMCPEGKAGLLCHLDDACISNPCHKGALCDTNPLNGQYICTCPQGYKGADCTEDVDECAMANSNPCEHAGKCVNTDGAFHCECLKGYAGPRCEMDINECHSDPCQNDATCLDKIGGFTCLCMPGFKGVHCELEINECQSNPCVNNGQCVDKVNRFQCLCPPGFTGPVCQIDIDDCSSTPCLNGAKCIDHPNGYECQCATGFTGVLCEENIDNCDPDPCHHGQCQDGIDSYTCICNPGYMGAICSDQIDECYSSPCLNDGRCIDLVNGYQCNCQPGTSGVNCEINFDDCASNPCIHGICMDGINRYSCVCSPGFTGQRCNIDIDECASNPCRKGATCINGVNGFRCICPEGPHHPSCYSQVNECLSNPCIHGNCTGGLSGYKCLCDAGWVGINCEVDKNECLSNPCQNGGTCDNLVNGYRCTCKKGFKGYNCQVNIDECASNPCLNQGTCFDDISGYTCHCVLPYTGKNCQTVLAPCSPNPCENAAVCKESPNFESYTCLCAPGWQGQRCTIDIDECISKPCMNHGLCHNTQGSYMCECPPGFSGMDCEEDIDDCLANPCQNGGSCMDGVNTFSCLCLPGFTGDKCQTDMNECLSEPCKNGGTCSDYVNSYTCKCQAGFDGVHCENNINECTESSCFNGGTCVDGINSFSCLCPVGFTGSFCLHEINECSSHPCLNEGTCVDGLGTYRCSCPLGYTGKNCQTLVNLCSRSPCKNKGTCVQKKAESQCLCPSGWAGAYCDVPNVSCDIAASRRGVLVEHLCQHSGVCINAGNTHYCQCPLGYTGSYCEEQLDECASNPCQHGATCSDFIGGYRCECVPGYQGVNCEYEVDECQNQPCQNGGTCIDLVNHFKCSCPPGTRGLLCEENIDDCARGPHCLNGGQCMDRIGGYSCRCLPGFAGERCEGDINECLSNPCSSEGSLDCIQLTNDYLCVCRSAFTGRHCETFVDVCPQMPCLNGGTCAVASNMPDGFICRCPPGFSGARCQSSCGQVKCRKGEQCVHTASGPRCFCPSPRDCESGCASSPCQHGGSCHPQRQPPYYSCQCAPPFSGSRCELYTAPPSTPPATCLSQYCADKARDGVCDEACNSHACQWDGGDCSLTMENPWANCSSPLPCWDYINNQCDELCNTVECLFDNFECQGNSKTCKYDKYCADHFKDNHCNQGCNSEECGWDGLDCAADQPENLAEGTLVIVVLMPPEQLLQDARSFLRALGTLLHTNLRIKRDSQGELMVYPYYGEKSAAMKKQRMTRRSLPGEQEQEVAGSKVFLEIDNRQCVQDSDHCFKNTDAAAALLASHAIQGTLSYPLVSVVSESLTPERTQLLYLLAVAVVIILFIILLGVIMAKRKRKHGSLWLPEGFTLRRDASNHKRREPVGQDAVGLKNLSVQVSEANLIGTGTSEHWVDDEGPQPKKVKAEDEALLSEEDDPIDRRPWTQQHLEAADIRRTPSLALTPPQAEQEVDVLDVNVRGPDGCTPLMLASLRGGSSDLSDEDEDAEDSSANIITDLVYQGASLQAQTDRTGEMALHLAARYSRADAAKRLLDAGADANAQDNMGRCPLHAAVAADAQGVFQILIRNRVTDLDARMNDGTTPLILAARLAVEGMVAELINCQADVNAVDDHGKSALHWAAAVNNVEATLLLLKNGANRDMQDNKEETPLFLAAREGSYEAAKILLDHFANRDITDHMDRLPRDVARDRMHHDIVRLLDEYNVTPSPPGTVLTSALSPVICGPNRSFLSLKHTPMGKKSRRPSAKSTMPTSLPNLAKEAKDAKGSRRKKSLSEKVQLSESSVTLSPVDSLESPHTYVSDTTSSPMITSPGILQASPNPMLATAAPPAPVHAQHALSFSNLHEMQPLAHGASTVLPSVSQLLSHHHIVSPGSGSAGSLSRLHPVPVPADWMNRMEVNETQYNEMFGMVLAPAEGTHPGIAPQSRPPEGKHITTPREPLPPIVTFQLIPKGSIAQPAGAPQPQSTCPPAVAGPLPTMYQIPEMARLPSVAFPTAMMPQQDGQVAQTILPAYHPFPASVGKYPTPPSQHSYASSNAAERTPSHSGHLQGEHPYLTPSPESPDQWSSSSPHSASDWSDVTTSPTPGGAGGGQRGPGTHMSEPPHNNMQVYA

SEQ ID NO: 69 - protein, 175 aa, Artificial Sequence
  Description of Artificial Sequence Synthetic EGF-like domain sequence
XXXXCXXXXXXXXXXXXXXXXXXXXXXXXXXXXXXXXXXXXXXXXXXXXXXXXCXXXXXXXXXXXXCXXXXXXXXXXXXXXXXXXXXXXXXXXXXXXXXXXXXXXXXXXXXXXXXXXXXXXXXXXXXXXXXXXXXXXCXXXXXXCXXGXXXXXXXXXXXXXXXXXXXXXXGXXCX